(19) United States
(12) Patent Application Publication
(10) Pub. No.: US 2014/0171429 A1
(43) Pub. Date: Jun. 19, 2014

(54) BICYCLIC INHIBITORS OF ALK

(71) Applicants: Anil Vasudevan, Union Grove, WI (US); Thomas Dale Penning, Elmhurst, IL (US); Huanming Chen, Irvine, CA (US); Bo Liang, Beijing (CN); Shaohui Wang, Beijing (CN); Zhongqiang Zhao, Beijing (CN); Dikun Chai, Beijing (CN); Leifu Yang, Beijing (CN); Yingxiang Gao, Beijing (CN); Marina Pliushchev, Vernon Hills, IL (US)

(72) Inventors: Anil Vasudevan, Union Grove, WI (US); Thomas Dale Penning, Elmhurst, IL (US); Huanming Chen, Irvine, CA (US); Bo Liang, Beijing (CN); Shaohui Wang, Beijing (CN); Zhongqiang Zhao, Beijing (CN); Dikun Chai, Beijing (CN); Leifu Yang, Beijing (CN); Yingxiang Gao, Beijing (CN); Marina Pliushchev, Vernon Hills, IL (US)

(73) Assignee: ABBVIE INC., North Chicago, IL (US)

(21) Appl. No.: 13/979,388

(22) PCT Filed: Jan. 20, 2012
(86) PCT No.: PCT/CN2012/000102

§ 371 Date: Feb. 19, 2014

(30) Foreign Application Priority Data
Jan. 21, 2011 (CN) PCT/CN2011/000110

(51) Int. Cl. C07D 471/04

(52) U.S. Cl. CPC C07D 471/04
USPC 514/234.5; 544/362; 514/253.04; 544/279; 514/252.16; 546/122; 514/300; 544/127

(57) ABSTRACT

The present invention relates to compounds of formula (1) or pharmaceutical acceptable salts, Formula (1) wherein R¹, R², R³, X, Y, Z, A, B, G¹, m, and n are defined in the description. The present invention relates also to compositions containing said compounds which are useful for inhibiting kinases such as ALK and methods of treating diseases such as cancer.

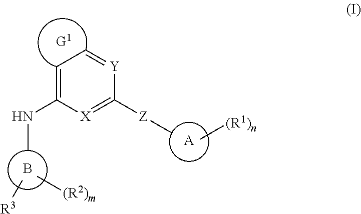

### FIELD OF THE INVENTION

This invention pertains to compounds which inhibit the activity of anaphastic lymphoma kinase (ALK), methods of making the compounds, compositions containing the compounds, and methods of treatment using the compounds.

### BACKGROUND OF THE INVENTION

Signaling through receptor tyrosine kinases (RTKs) regulates and fine-tunes many processes including cell growth, proliferation, differentiation, and apoptosis. The improper activation of RTKs is involved in the pathogenesis, growth, and metastasis of many cancers. The receptor tyrosine kinase ALK (Anaplastic Lymphoma Kinase) is a member of the insulin receptor superfamily that was initially identified from the t(2;5)(p23;q35) translocation in anaplastic large cell lymphoma (ALCL) (Fischer, P., et al. Blood, 72: 234-240. (1988)). The protein product of this translocation is ALK fused to nucleophosmin (NPM) (Morris et al., 1994). When fused to ALK, the dimerization domain of NPM results in constitutive dimerization and activation of ALK (reviewed in Chiarle, R., Nature reviews, 8:11-23 (2008)). Once activated, ALK recruits several adaptor proteins and stimulates multiple signaling pathways known to mediate tumor cell growth and survival including STAT3, PLC-γ, RAS-ERK1,2, and PI3K-AKT (Bai, R. Y., et al. Molecular and cellular biology 18: 6951-6961 (1998); Bai, R. Y., et al. Blood 96:4319-4327 (2000); Chiarle, R., et al. Nature medicine 11:623-629 (2005); Pulford, K., et al. Journal of cellular physiology 199:330-358 (2004)). The dysregulation of ALK is highly oncogenic, as it is sufficient to induce cell transformation in a several immortalized cell lines (Bischof, D., et al. Molecular and cellular biology 17:2312-2325 (199.7); Fujimoto, J., et al. Proceedings of the National Academy of Sciences of the United. States of America 93: 4181-4186 (1996)) and to form tumors in animal models (Chiarle, R., et al. Blood 101: 1919-1927 (2003); Kuefer, M. U., et al: Blood 90: 2901-2910 (1997)). Moreover, NPM-ALK drives tumor formation, proliferation and survival in ALCL (reviewed in (Duyster, J., et al. Oncogene 20: 5623-5637 (2001)).

More recently, ALK translocations have been detected in 5% of non-small cell lung cancers (NSCLC). Similar to ALK translocations in ALCL, the fusion proteins in NSCLC display constitutive ALK activity and drive tumor growth and survival (Soda et al., Nature 448: 561-566 (2007); Soda et al., Proceedings of the National Academy of Sciences of the United States of America 105: 19893-19897 (2008)). NSCLC tumors harboring ALK translocations are mutually exclusive from K-Ras or EGFR aberrations and predominantly occur in younger patients that are non-smokers (Rodig et al., Clin Cancer Res 15: 5216-5223 (2009); Shaw et al., J Clin Oncol 27: 4247-4253 (2009); Wong et al., Cancer 115: 1723-1733 (2009)). In addition to chromosomal rearrangements, activating point mutations and amplifications have been reported in a subset of sporadic and familial neuroblastomas, further expanding the spectrum of tumors dependent on ALK activity (Chen et al., Nature 455: 971-974 (2008); George et al., Nature 455: 975-978 (2008); Janoueix-Lerosey et al., Nature 455: 967-970 (2008); Mosse et al., Nature 455: 930-935 (2008)). Neuroblastomas with ALK genetic aberrations also are dependent on ALK for proliferation and survival, and cells expressing ALK containing activating mutations form tumors in animal models.

Inhibitors of RTKs have the potential to cause lethality in cancerous cells that are reliant on deregulated RTK activity while sparing normal tissues. Thus, small molecule inhibitors of ALK would be beneficial for therapeutic intervention in ALCL, NSCLC, neuroblastoma, and other cancers that are dependent on ALK for growth and survival.

### SUMMARY OF THE INVENTION

The present invention has numerous embodiments. One embodiment of this invention, therefore, pertains to compounds that have formula (I)

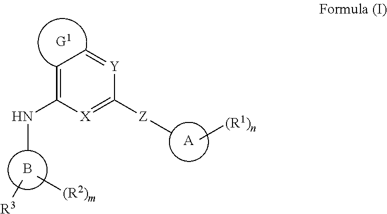

wherein R¹, R², R³, X, Y, Z, A, B, G¹, m, and n are as defined below and subsets therein.

Also provided are pharmaceutically acceptable compositions, comprising a therapeutically effective amount of a compound of formula (I) and a pharmaceutically acceptable salt in combination with a pharmaceutically suitable carrier.

One embodiment is directed to a method of treating cancer in a mammal comprising administering thereto a therapeutically acceptable amount of a compound or pharmaceutically acceptable salt of formula (I). Another embodiment pertains to a method of decreasing tumor volume in a mammal comprising administering thereto a therapeutically acceptable amount of a compound or pharmaceutically acceptable salt of formula (I).

### DETAILED DESCRIPTION OF THE INVENTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### ABBREVIATIONS AND DEFINITIONS

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. In this application, the use of “or” means “and/or” unless stated otherwise. Furthermore, the use of the term “including”, as well as other forms, such as “includes” and “included”, is not limiting. With reference to the use of the words “comprise” or “comprises” or “comprising” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below. For a variable that occurs more than one time in any substituent or in the compound of the invention or any other formulae herein, its definition on each occurrence is independent of its definition at every other occurrence. Combinations of substituents are permissible only if such combinations result in stable compounds. Stable compounds are compounds which can be isolated in a useful degree of purity from a reaction mixture.

It is meant to be understood that proper valences are maintained for all combinations herein, that monovalent moieties having more than one atom are attached through their left ends, and that divalent moieties are drawn from left to right.

As used in the specification and the appended claims, unless specified to the contrary, the following terms have the meaning indicated:

The term “alkyl” (alone or in combination with another term(s)) means a straight- or branched-chain saturated hydrocarbyl substituent typically containing from 1 to about 10 carbon atoms; or in another embodiment, from 1 to about 8 carbon atoms; in another embodiment, from 1 to about 6 carbon atoms; and in another embodiment, from 1 to about 4 carbon atoms. Examples of such substituents include methyl, ethyl, n-propyl, isopropyl, n-butyl, isobutyl, sec-butyl, tert-butyl, pentyl, iso-amyl, and hexyl and the like.

The term “alkenyl” (alone or in combination with another term(s)) means a straight- or branched-chain hydrocarbyl substituent containing one or more double bonds and typically from 2 to about 10 carbon atoms; or in another embodiment, from 2 to about 8 carbon atoms; in another embodiment, from 2 to about 6 carbon atoms; and in another embodiment, from 2 to about 4 carbon atoms. Examples of such substituents include ethenyl(vinyl), 2-propenyl, 3-propenyl, 1,4-pentadienyl, 1,4-butadienyl, 1-butenyl, 2-butenyl, and 3-butenyl and the like.

The term “alkynyl” (alone or in combination with another term(s)) means a straight- or branched-chain hydrocarbyl substituent containing one or more triple bonds and typically from 2 to about 10 carbon atoms; or in another embodiment, from 2 to about 8 carbon atoms; in another embodiment, from 2 to about 6 carbon atoms; and in another embodiment, from 2 to about 4 carbon atoms. Examples of such substituents include ethynyl, 2-propynyl, 3-propynyl, 2-butynyl, and 3-butynyl and the like.

The term “carbocyclyl” (alone or in combination with another term(s)) means a saturated cyclic (i.e., “cycloalkyl”), partially saturated cyclic (i.e., “cycloalkenyl”), or completely unsaturated (i.e., “aryl”) hydrocarbyl substituent containing from 3 to 14 carbon ring atoms (“ring atoms” are the atoms bound together to form the ring or rings of a cyclic substituent). A carbocyclyl may be a single-ring (monocyclic) or polycyclic ring structure.

A carbocyclyl may be a single ring structure, which typically contains from 3 to 8 ring atoms, more typically from 3 to 6 ring atoms, and even more typically 5 to 6 ring atoms. Examples of such single-ring carbocyclyls include cyclopropyl(cyclopropanyl), cyclobutyl(cyclobutanyl), cyclopentyl(cyclopentanyl), cyclopentenyl, cyclopentadienyl, cyclohexyl(cyclohexanyl), cyclohexenyl, cyclohexadienyl, and phenyl. A carbocyclyl may alternatively be polycyclic (i.e., may contain more than one ring). Examples of polycyclic carbocyclyls include bridged, fused, and spirocyclic carbocyclyls. In a spirocyclic carbocyclyl, one atom is common to two different rings. An example of a spirocyclic carbocyclyl is spiropentanyl. In a bridged carbocyclyl, the rings share at least two common non-adjacent atoms. Examples of bridged carbocyclyls include bicyclo[2.2.1]heptanyl, bicyclo[2.2.1]hept-2-enyl, and adamantanyl. In a fused-ring carbocyclyl system, two or more rings may be fused together, such that two rings share one common bond. Examples of two- or three-fused ring carbocyclyls include naphthalenyl, tetrahydronaphthalenyl(tetralinyl), indenyl, indanyl(dihydroindenyl), anthracenyl, phenanthrenyl, and decalinyl.

The term “cycloalkyl” (alone or in combination with another term(s)) means a saturated cyclic hydrocarbyl substituent containing from 3 to 14 carbon ring atoms. A cycloalkyl may be a single carbon ring, which typically contains from 3 to 8 carbon ring atoms and more typically from 3 to 6 ring atoms. Examples of single-ring cycloalkyls include cyclopropyl, cyclobutyl, cyclopentyl, and cyclohexyl. A cycloalkyl may alternatively be polycyclic or contain more than one ring. Examples of polycyclic cycloalkyls include bridged, fused, and spirocyclic carbocyclyls.

The term “aryl” (alone or in combination with another term(s)) means an aromatic carbocyclyl containing from 6 to 14 carbon ring atoms. An aryl may be monocyclic or polycyclic (i.e., may contain more than one ring). In the case of polycyclic aromatic rings, only one ring the polycyclic system is required to be unsaturated while the remaining ring(s) may be saturated, partially saturated or unsaturated. Examples of aryls include phenyl, naphthalenyl, indenyl, indanyl, and tetrahydronapthyl.

In some instances, the number of carbon atoms in a hydrocarbyl substituent (e.g., alkyl, alkenyl, alkynyl, or cycloalkyl) is indicated by the prefix “Cₓ—C_{y}-”, wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₈-cycloalkyl means a saturated hydrocarbyl ring containing from 3 to 8 carbon ring atoms.

The term “hydrogen” (alone or in combination with another term(s)) means a hydrogen radical, and may be depicted as —H.

The term “hydroxy” (alone or in combination with another term(s)) means —OH.

The term “carboxy” (alone or in combination with another term(s)) means —C(O)—OH.

The term “amino” (alone or in combination with another term(s)) means —NH₂.

The term “halogen” or “halo” (alone or in combination with another term(s)) means a fluorine radical (which may be depicted as —F), chlorine radical (which may be depicted as —Cl), bromine radical (which may be depicted as —Br), or iodine radical (which may be depicted as —I).

If a substituent is described as being “substituted”, a non-hydrogen radical is in the place of hydrogen radical on a carbon or nitrogen of the substituent. Thus, for example, a substituted alkyl substituent is an alkyl substituent in which at least one non-hydrogen radical is in the place of a hydrogen radical on the alkyl substituent. To illustrate, monofluoroalkyl is alkyl substituted with a fluoro radical, and difluoroalkyl is alkyl substituted with two fluoro radicals. It should be recognized that if there are more than one substitution on a substituent, each non-hydrogen radical may be identical or different (unless otherwise stated).

If a substituent is described as being “optionally substituted”, the substituent may be either (1) not substituted or (2) substituted. If a substituent is described as being optionally substituted with up to a particular number of non-hydrogen radicals, that substituent may be either (1) not substituted; or (2) substituted by up to that particular number of non-hydrogen radicals or by up to the maximum number of substitutable positions on the substituent, whichever is less. Thus, for example, if a substituent is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

This patent application uses the terms “substituent” and “radical” interchangeably.

The prefix “halo” indicates that the substituent to which the prefix is attached is substituted with one or more independently selected halogen radicals. For example, haloalkyl means an alkyl substituent in which at least one hydrogen radical is replaced with a halogen radical. Examples of haloalkyls include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated).

The prefix “perhalo” indicates that every hydrogen radical on the substituent to which the prefix is attached is replaced with independently selected halogen radicals, i.e., each hydrogen radical on the substituent is replaced with a halogen radical. If all the halogen radicals are identical, the prefix typically will identify the halogen radical. Thus, for example, the term “perfluoro” means that every hydrogen radical on the substituent to which the prefix is attached is substituted with a fluorine radical. To illustrate, the term “perfluoroalkyl” means an alkyl substituent wherein a fluorine radical is in the place of each hydrogen radical.

The term “carbonyl” (alone or in combination with another term(s)) means —C(O)—.

The term “aminocarbonyl” (alone or in combination with another term(s)) means —C(O)—NH₂.

The term “oxo” (alone or in combination with another term(s)) means (═O).

The term “oxy” (alone or in combination with another term(s)) means an ether substituent, and may be depicted as —O—.

The term “alkylhydroxy” (alone or in combination with another term(s)) means alkyl-OH.

The term “alkylamino” (alone or in combination with another term(s)) means alkyl-NH₂.

The term “alkyloxy” (alone or in combination with another term(s)) means an alkylether substituent, i.e., —O-alkyl. Examples of such a substituent include methoxy (—O—CH₃), ethoxy, n-propoxy, isopropoxy, n-butoxy, iso-butoxy, sec-butoxy, and tert-butoxy.

The term “alkylcarbonyl” (alone or in combination with another term(s)) means —C(O)-alkyl.

The term “aminoalkylcarbonyl” (alone or in combination with another term(s)) means —C(O)-alkyl-NH₂.

The term “alkyloxycarbonyl” (alone or in combination with another term(s)) means —C(O)—O-alkyl.

The term “carbocyclylcarbonyl” (alone or in combination with another term(s)) means —C(O)-carbocyclyl.

Similarly, the term “heterocyclylcarbonyl” (alone or in combination with another term(s)) means —C(O)-heterocyclyl.

The term “carbocyclylalkylcarbonyl” (alone or in combination with another term(s)) means —C(O)-alkyl-carbocyclyl.

Similarly, the term “heterocyclylalkylcarbonyl” (alone or in combination with another term(s)) means —C(O)-alkyl-heterocyclyl.

The term “carbocyclyloxycarbonyl” (alone or in combination with another term(s)) means —C(O)—O-carbocyclyl.

The term “carbocyclylalkyloxycarbonyl” (alone or in combination with another term(s)) means —C(O)—O-alkyl-carbocyclyl.

The term “thio” or “thia” (alone or in combination with another term(s)) means a thiaether substituent, i.e., an ether substituent wherein a divalent sulfur atom is in the place of the ether oxygen atom. Such a substituent may be depicted as —S—. This, for example, “alkyl-thio-alkyl” means alkyl-S-alkyl (alkyl-sulfanyl-alkyl).

The term “thiol” or “sulfhydryl” (alone or in combination with another term(s)) means a sulfhydryl substituent, and may be depicted as —SH.

The term “(thiocarbonyl)” (alone or in combination with another term(s)) means a carbonyl wherein the oxygen atom has been replaced with a sulfur. Such a substituent may be depicted as —C(S)—.

The term “sulfonyl” (alone or in combination with another term(s)) means —S(O)₂—.

The term “aminosulfonyl” (alone or in combination with another term(s)) means —S(O)₂—NH₂.

The term “sulfinyl” or “sulfoxido” (alone or in combination with another term(s)) means —S(O)—.

The term “heterocyclyl” (alone or in combination with another term(s)) means a saturated (i.e., “heterocycloalkyl”), partially saturated (i.e., “heterocycloalkenyl”), or completely unsaturated (i.e., “heteroaryl”) ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur. A heterocyclyl may be a single-ring (monocyclic) or polycyclic ring structure.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, dihydrofuranyl, tetrahydrofuranyl, thiophenyl(thiofuranyl), dihydrothiophenyl, tetrahydrothiophenyl, pyrrolyl, pyrrolinyl, pyrrolidinyl, imidazolyl, imidazolinyl, imidazolidinyl, pyrazolyl, pyrazolinyl, pyrazolidinyl, triazolyl, tetrazolyl, oxazolyl, oxazolidinyl, isoxazolidinyl, isoxazolyl, thiazolyl, isothiazolyl, thiazolinyl, isothiazolinyl, thiazolidinyl, isothiazolidinyl, thiodiazolyl, oxadiazolyl (including 1,2,3-oxadiazolyl, 1,2,4-oxadiazolyl, 1,2,5-oxadiazolyl(furazanyl), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl, dihydropyranyl, thiopyranyl, tetrahydrothiopyranyl, pyridinyl(azinyl), piperidinyl, diazinyl (including pyridazinyl(1,2-diazinyl), pyrimidinyl(1,3-diazinyl), or pyrazinyl(1,4-diazinyl)), piperazinyl, triazinyl (including 1,3,5-triazinyl, 1,2,4-triazinyl, and 1,2,3-triazinyl)), oxazinyl (including 1,2-oxazinyl, 1,3-oxazinyl, or 1,4-oxazinyl)), oxathiazinyl (including 1,2,3-oxathiazinyl, 1,2,4-oxathiazinyl, 1,2,5-oxathiazinyl, or 1,2,6-oxathiazinyl)), oxadiazinyl (including 1,2,3-oxadiazinyl, 1,2,4-oxadiazinyl, 1,4,2-oxadiazinyl, or 1,3,5-oxadiazinyl)), morpholinyl, azepinyl, oxepinyl, thiepinyl, and diazepinyl.

A heterocyclyl may alternatively be polycyclic (i.e., may contain more than one ring). Examples of polycyclic heterocyclyls include bridged, fused, and spirocyclic heterocyclyls. In a spirocyclic heterocyclyl, one atom is common to two different rings. In a bridged heterocyclyl, the rings share at least two common non-adjacent atoms. In a fused-ring heterocyclyl, two or more rings may be fused together, such that two rings share one common bond. Examples of fused ring heterocyclyls containing two or three rings include indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl.

Other examples of fused-ring heterocyclyls include benzo-fused heterocyclyls, such as indolyl, isoindolyl(isobenzazolyl, pseudoisoindolyl), indoleninyl(pseudoindolyl), isoindazolyl(benzpyrazolyl), benzazinyl (including quinolinyl(1-benzazinyl) or isoquinolinyl(2-benzazinyl)), phthalazinyl, quinoxalinyl, quinazolinyl, benzodiazinyl (including cinnolinyl(1,2-benzodiazinyl) or quinazolinyl(1,3-benzodiazinyl)), benzopyranyl (including chromanyl or isochromanyl), benzoxazinyl (including 1,3,2-benzoxazinyl, 1,4,2-benzoxazinyl, 2,3,1-benzoxazinyl, or 3,1,4-benzoxazinyl), and benzisoxazinyl (including 1,2-benzisoxazinyl or 1,4-benzisoxazinyl).

The term “heterocycloalkyl” (alone or in combination with another term(s)) means a saturated heterocyclyl.

The term “heteroaryl” (alone or in combination with another term(s)) means an aromatic heterocyclyl containing from 5 to 14 ring atoms. A heteroaryl may be a single ring or 2 or 3 fused rings. Examples of heteroaryl substituents include 6-membered ring substituents such as pyridyl, pyrazyl, pyrimidinyl, pyridazinyl, and 1,3,5-, 1,2,4- or 1,2,3-triazinyl; 5-membered ring substituents such as imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, benzisoxazolyl, benzoxazolyl, purinyl, and anthranilyl; and 6/6-membered fused rings such as benzopyranyl, quinolinyl, isoquinolinyl, cinnolinyl, quinazolinyl, and benzoxazinyl.

A prefix attached to a multi-component substituent only applies to the first component. To illustrate, the term “alkylcycloalkyl” contains two components: alkyl and cycloalkyl. Thus, the C₁-C₆-prefix on C₁-C₆-alkylcycloalkyl means that the alkyl component of the alkylcycloalkyl contains from 1 to 6 carbon atoms; the C₁-C₆-prefix does not describe the cycloalkyl component. To illustrate further, the prefix “halo” on haloalkyloxyalkyl indicates that only the alkyloxy component of the alkyloxyalkyl substituent is substituted with one or more halogen radicals. If halogen substitution may alternatively or additionally occur on the alkyl component, the substituent would instead be described as “halogen-substituted alkyloxyalkyl” rather than “haloalkyloxyalkyl.” And finally, if the halogen substitution may only occur on the alkyl component, the substituent would instead be described as “alkyloxyhaloalkyl.”

The terms “treat”, “treating” and “treatment” refer to a method of alleviating or abrogating a disease and/or its attendant symptoms.

The terms “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

The term “therapeutically effective amount” refers to that amount of the compound being administered sufficient to prevent development of or alleviate to some extent one or more of the symptoms of the condition or disorder being treated.

The term “modulate” refers to the ability of a compound to increase or decrease the function, or activity, of a kinase. “Modulation”, as used herein in its various forms, is intended to encompass antagonism, agonism, partial antagonism and/or partial agonism of the activity associated with kinase. Kinase inhibitors are compounds that, e.g., bind to, partially or totally block stimulation, decrease, prevent, delay activation, inactivate, desensitize, or down regulate signal transduction. Kinase activators are compounds that, e.g., bind to, stimulate, increase, open, activate, facilitate, enhance activation, sensitize or up regulate signal transduction.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The “subject” is defined herein to include animals such as mammals, including, but not limited to, primates (e.g., humans), cows, sheep, goats, horses, dogs, cats, rabbits, rats, mice and the like. In preferred embodiments, the subject is a human.

### Compounds

### Embodiments of Formula (I)

In one embodiment, the present invention is directed, in part, to a class of compounds having a structure of Formula I

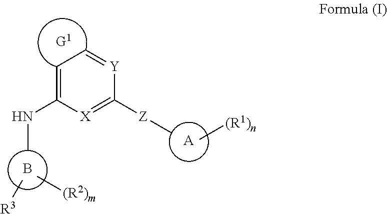

wherein

G¹ is

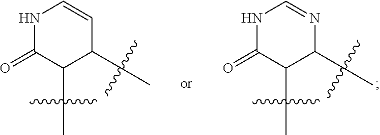 [SMILES: *C1C=CNC(=O)C1* ; *C1N=CNC(=O)C1*]

X is CH or N;

Y is CH or N;

wherein at least one of X and Y is N;

A is phenyl, naphthyl, indenyl, C₃₋₈ cycloalkyl, 5-7 membered heterocycloalkyl, 5-7 membered heterocycloalkenyl, or 5-7 membered heteroaryl;

B is phenyl, pyridinyl, pyrimidinyl, pyrazinyl, pyridazinyl, or pyrazolinyl;

Z is C₁₋₆ alkylene;

R¹, at each occurrence, is independently selected from the group consisting of halo, CN, NO₂, C₁₋₆-haloalkyl, aryl, C₃₋₈ cycloalkyl, heteroaryl, heterocycloalkyl, OR⁵, SR⁵, C(O)R⁵, C(O)NR⁶R⁷, C(O)OR⁵, OC(O)R⁵, OC(O)NR⁶R⁷, NR⁶R⁷, NR⁶C(O)R⁵, S(O)R⁵, S(O)NR⁶R⁷, S(O)₂R⁵, NR⁶S(O)₂R⁵, and S(O)₂NR⁶R⁷; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c};

R², at each occurrence, is independently selected from the group consisting of halo, CN, OH, C₁₋₄ alkyl, C₁₋₄-haloalkyl, C₁₋₄ alkoxy, C₁₋₄ haloalkoxy, C₁₋₄-thioalkoxy, amino, C₁₋₄ alkylamino, and C₁₋₄ dialkylamino;

R³ is selected from the group consisting of aryl, C₃₋₈ cycloalkyl, heteroaryl, heterocycloalkyl, heteroaryl-C₁₋₆-alkyl-, heterocycloalkyl-C₁₋₆-alkyl-, OR⁸, C(O)R⁸, C(O)NR⁹R¹⁰, C(O)OR⁸, OC(O)R⁸, OC(O)NR⁹R¹⁰, NR⁹R¹⁰, NR⁹C(O)R⁸, S(O)R⁸, S(O)NR⁹R¹⁰, S(O)₂R⁸, NR⁹S(O)₂R⁸, and S(O)₂NR⁹R¹⁰, wherein the C₃₋₈ cycloalkyl, aryl, heterocycloalkyl, and heteroaryl, alone or part of another moiety, are optionally substituted with one, two, or three R¹¹;

R⁴ is H or C₁₋₆-alkyl;

R⁵, R⁶, and R⁷, at each occurrence, are independently selected from H, C₁₋₆ alkyl, C₁₋₆ haloalkyl, aryl, C₃₋₈ cycloalkyl, heteroaryl, and heterocycloalkyl, wherein the aryl, C₃₋₈ cycloalkyl, heteroaryl, and heterocycloalkyl moiety are optionally substituted with 1, 2, or 3 substituents independently selected from halo, CN, OH, C₁₋₄ alkyl, C₁₋₄-haloalkyl, C₁₋₄ alkoxy, C₁₋₄ haloalkoxy, amino, C₁₋₄ alkylamino, C₁₋₄ dialkylamino, C(O)OH, C(O)C₁₋₄ alkyl, C(O)NH₂, C(O)NH(C₁₋₄ alkyl), or C(O)N(C₁₋₄ alkyl)₂;

R⁸, R⁹, and R¹⁰, at each occurrence, are independently selected from H, C₁₋₆ alkyl, C₁₋₆ haloalkyl, heteroaryl-C₁₋₆-alkyl-, heterocycloalkyl-C₁₋₆-alkyl-, R¹²R¹³N—C₁₋₆-alkyl-, aryl, C₃₋₈ cycloalkyl, heteroaryl, and heterocycloalkyl, wherein the aryl, C₃₋₈ cycloalkyl, heteroaryl, and heterocycloalkyl, alone or as part of another moiety, are optionally substituted with 1, 2, or 3 substituents independently selected from halo, CN, OH, C₁₋₄ alkyl, C₁₋₄-haloalkyl, C₁₋₄ alkoxy, C₁₋₄ haloalkoxy, amino, C₁₋₄ alkylamino, C₁₋₄ dialkylamino, C(O)OH, C(O)C₁₋₄ alkyl, C(O)NH₂, C(O)NH(C₁₋₄ alkyl), or C(O)N(C₁₋₄ alkyl)₂;

R¹¹, at each occurrence, is independently selected from the group consisting of halo, C₁₋₄ alkyl, C₁₋₄ haloalkyl, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl;

R¹² and R¹³, at each occurrence, are independently selected from the group consisting of H, C₁₋₆ alkyl, C₁₋₆ haloalkyl, aryl, C₃₋₈ cycloalkyl, heteroaryl, and heterocycloalkyl;

R^{a}, at each occurrence, is independently selected from the group consisting of H, C₁₋₆ alkyl, aryl, C₃₋₈ cycloalkyl, heteroaryl, and heterocycloalkyl; wherein the C₁₋₆alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{b} and R^{c}, at each occurrence, are independently selected from the group consisting of H, C₁₋₆ alkyl, aryl, C₃₋₈ cycloalkyl, heteroaryl, and heterocycloalkyl; wherein the C₁₋₆ alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{d}, at each occurrence, is independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{e} and R^{f}, at each occurrence, are independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{g}, at each occurrence, is independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

R^{h} and Rⁱ, at each occurrence, are independently selected from the group consisting of H, C₁₋₆ alkyl, aryl, C₃₋₈ cycloalkyl, heteroaryl, and heterocycloalkyl; wherein the C₁₋₆ alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;

m is 0, 1, 2, or 3; and

n is 1, 2, or 3;

or a pharmaceutically acceptable salt or solvate thereof.

In one embodiment of formula (I), G¹ is

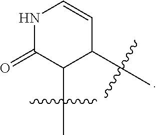 [SMILES: *C1C=CNC(=O)C1*]

In another embodiment of formula (I), G¹ is

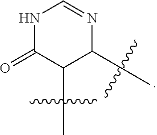 [SMILES: *C1N=CNC(=O)C1*]

In one embodiment of formula (I), X is N; and Y is CH. In another embodiment of formula (I), X is CH; and Y is N. In another embodiment of formula (I), X is N; and Y is N.

In another embodiment of formula (I), G¹ is

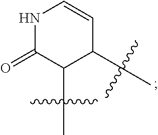 [SMILES: *C1C=CNC(=O)C1*]

X is CH; and Y is N. In another embodiment of formula (I), G¹ is

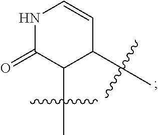 [SMILES: *C1C=CNC(=O)C1*]

X is N; and Y is CH. In another embodiment of formula (I), G¹ is

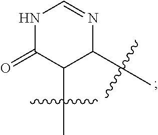 [SMILES: *C1N=CNC(=O)C1*]

### X is N; and Y is CH.

In one embodiment of formula (I), Z is C₁₋₆ alkylene. In another embodiment of formula (I), Z is —CH₂—, —CH₂CH₂—, —CH₂CH₂CH₂—, or —CH₂CH₂CH₂CH₂—. In another embodiment of formula (I), Z is —CH(CH₃)—, —CH₂CH(CH₃)—, —CH(CH₃)CH₂—, —CH(CH₃)CH₂CH₂—, —CH₂CH(CH₃)CH₂—, —CH₂CH₂CH(CH₃)—, —C(CH₃)₂—, —CH₂C(CH₃)₂—, —C(CH₃)₂CH₂—, —CH₂CH₂C(CH₃)₂—, —CH₂C(CH₃)₂CH₂—, or —C(CH₃)₂CH₂CH₂—. In another embodiment of formula (I), Z is CH(CH₂CH₃)—, —CH₂CH(CH₂CH₃)—, —CH(CH₂CH₃)CH₂—, —CH(CH₂CH₃)CH₂CH₂—, —CH₂CH(CH₂CH₃)CH₂—, —CH₂CH₂CH(CH₂CH₃)—, —C(CH₂CH₃)₂—, —CH₂C(CH₂CH₃)₂—, —C(CH₂CH₃)₂CH₂—, —CH₂CH₂C(CH₂CH₃)₂—, —CH₂C(CH₂CH₃)₂CH₂—, or —C(CH₂CH₃)₂CH₂CH₂—. In yet another embodiment of formula (I), Z is —CH₂—, —CH₂CH₂—. —CH(CH₃)—, or —C(CH₃)₂—. In yet another embodiment of formula (I), Z is —CH₂—.

In one embodiment of formula (I), A is phenyl, naphthyl, indenyl or C₃₋₈ cycloalkyl. In yet another embodiment of formula (I), A is phenyl.

In another embodiment of formula (I), A is a 5-7 membered heterocycloalkyl or heterocycloalkenyl. In another embodiment of formula (I), A is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (I), A is dihydrofuranyl, dihydrothiophenyl, pyrrolinyl, imidazolinyl, pyrazolinyl, thiazolinyl, isothiazolinyl, dihydropyranyl, oxathiazinyl, oxadiazinyl, or oxazinyl.

In one embodiment of formula (I), A is a 5-7 membered heteroaryl. In another embodiment of formula (I), A is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl.

In one embodiment of formula (I), A is optionally substituted with —(R¹)ₙ, wherein n is 0, 1, 2, or 3. In one embodiment of formula (I), R¹, at each occurrence, is independently selected from the group consisting of halo, CN, NO₂, C₁₋₆-haloalkyl, aryl, C₃₋₈ cycloalkyl, heteroaryl, heterocycloalkyl, OR⁵, SR⁵, C(O)R⁵, C(O)NR⁶R⁷, C(O)OR⁵, OC(O)R⁵, OC(O)NR⁶R⁷, NR⁶R⁷, NR⁶C(O)R⁵, S(O)R⁵, S(O)NR⁶R⁷, S(O)₂R⁵, NR⁶S(O)₂R⁵, and S(O)₂NR⁶R⁷; wherein the C₃₋₈ cycloalkyl, aryl, heterocycloalkyl, and heteroaryl are optionally substituted with 1, 2, or 3 substituents independently selected from halo, C₁₋₄ alkyl. C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c}.

In another embodiment of formula (I), A is phenyl, n is 2, and R¹, at each occurrence, is halo.

In one embodiment of formula (I), B is phenyl, pyridinyl, pyrimidinyl, pyrazinyl, pyridazinyl, or pyrazolinyl. In another embodiment of formula (I), B is phenyl.

In one embodiment of formula (I), B is

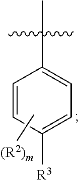

wherein R², R³, and m are as defined above. In another embodiment of formula (I), m is 0. In another embodiment of formula (I), m is 1, and R², at each occurrence, is independently selected from the group consisting of halo, CN, OH, C₁₋₄ alkyl, C₁₋₄-haloalkyl, C₁₋₄ alkoxy, C₁₋₄ haloalkoxy, C₁₋₄-thioalkoxy, amino, C₁₋₄-alkylamino, and C₁₋₄ dialkylamino. In yet another embodiment of formula (I), m is 1 and R² is selected from the group consisting of halo, and C₁₋₄ alkoxy. In another embodiment of formula (I), R³ is selected from the group consisting aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁸, C(O)R⁸, C(O)NR⁹R¹⁰, C(O)OR⁸, OC(O)R⁸, OC(O)NR⁹R¹⁰, NR⁹R¹⁰, NR⁹C(O)R⁸, S(O)R⁸, S(O)NR⁹R¹⁰, S(O)₂R⁸, NR⁹S(O)₂R⁸, and S(O)₂NR⁹R¹⁰, wherein the C₃₋₈ cycloalkyl, aryl, heterocycloalkyl, and heteroaryl, alone or part of another moiety, are optionally substituted with one, two, or three R¹¹, wherein R¹¹ is defined above.

In yet another embodiment of formula (I), B is phenyl, and R³ is heterocycloalkyl. In yet another embodiment of formula (I), R³ is heterocycloalkyl. In yet another embodiment of formula (I), R³ is heterocycloalkyl, which is optionally substituted with one R¹¹, and R¹¹ is selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{d}, R^{e}, and R^{f} are as defined above. In yet another embodiment of formula (I), R³ is heterocycloalkyl, which is optionally substituted with one R¹¹, and R¹¹ is selected from the group consisting of C₁₋₄ alkyl and NR^{e}R^{f}. In yet another embodiment of formula (I), R³ is heterocycloalkyl, which is optionally substituted with one R¹¹, and R¹¹ is C₁₋₄ alkyl. In yet another embodiment of formula (I), R³ is heterocycloalkyl, which is optionally substituted with one R¹¹, and R¹¹ is NR^{e}R^{f}, wherein R^{e} and R^{f} are C₁₋₆ alkyl.

In one embodiment of formula (I), B is

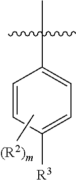

m is 0 or 1;
R² is halo or C₁₋₄ alkoxy;

### R³ is

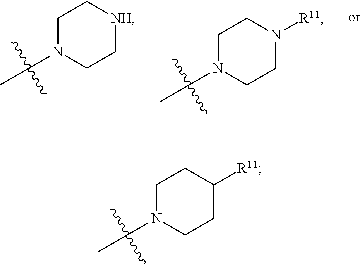

and
R¹¹ is halo, C₁₋₄ alkyl, C₁₋₄ haloalkyl, amino-C₁₋₄-alkyl-, C₁₋₄-alkylamino-C₁₋₄ alkyl-, C₁₋₄ dialkylamino-C₁₋₄ hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl.

In another embodiment of formula (I), B is

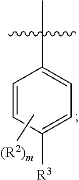

m is 0 or 1;
R² is halo, or C₁₋₄ alkoxy;

### R³ is

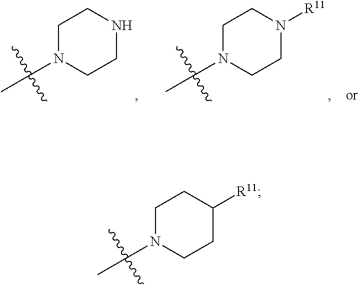

and
R¹¹ is C₁₋₄ alkyl, or NR^{e}R^{f}.

In another embodiment of formula (I), B is

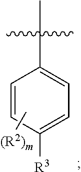

m is 0 or 1;
R² is halo, or C₁₋₄ alkoxy;

### R³ is

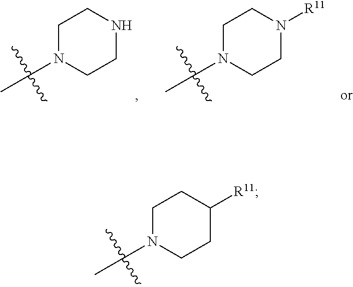

R¹¹ is C₁₋₄ alkyl, or NR^{e}R^{f}; and
R^{e} and R^{f} are C₁₋₆ alkyl.

### Embodiments of Formula (II)

In one embodiment of formula (II), the present invention is directed, in part, to a class of compounds having a structure of Formula (II)

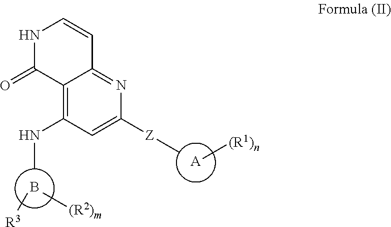

wherein R¹, R², R³, A, B, Z, m, and n are as described in formula (I).

In one embodiment of formula (II), Z is C₁₋₆ alkylene. In another embodiment of formula (II), Z is —CH₂—, —CH₂CH₂—, —CH₂CH₂CH₂—, or —CH₂CH₂CH₂CH₂—, In another embodiment of formula (II), Z is —CH(CH₃)—, —CH₂CH(CH₃)—, —CH(CH₃)CH₂—, —CH(CH₃)CH₂CH₂—, —CH₂CH(CH₃)CH₂—, —CH₂CH₂CH(CH₃)—, —C(CH₃)₂—, —CH₂C(CH₃)₂—, —C(CH₃)₂CH₂—, —CH₂CH₂C(CH₃)₂—, —CH₂C(CH₃)₂CH₂—, or —C(CH₃)₂CH₂CH₂—. In another embodiment of formula (II), Z is CH(CH₂CH₃)—, —CH₂CH(CH₂CH₃)—, —CH(CH₂CH₃)CH₂—, —CH(CH₂CH₃)CH₂CH₂—, —CH₂CH(CH₂CH₃)CH₂—, —CH₂CH₂CH(CH₂CH₃)—, —C(CH₂CH₃)₂—, —CH₂C(CH₂CH₃)₂—, —C(CH₂CH₃)₂CH₂—, —CH₂CH₂C(CH₂CH₃)₂—, —CH₂C(CH₂CH₃)₂CH₂—, or —C(CH₂CH₃)₂CH₂CH₂—. In yet another embodiment of formula (II), Z is —CH₂—, —CH₂CH₂—, —CH(CH₃)—, or —C(CH₃)₂—. In yet another embodiment of formula (II), Z is —CH₂—.

In one embodiment of formula (II), A is phenyl, naphthyl, indenyl or C₃₋₈ cycloalkyl. In yet another embodiment of formula (II), A is phenyl.

In another embodiment of formula (II), A is a 5-7 membered heterocycloalkyl or heterocycloalkenyl. In another embodiment of formula (II), A is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (II), A is dihydrofuranyl, dihydrothiophenyl, pyrrolinyl, imidazolinyl, pyrazolinyl, thiazolinyl, isothiazolinyl, dihydropyranyl, oxathiazinyl, oxadiazinyl, or oxazinyl.

In one embodiment of formula (II), A is a 5-7 membered heteroaryl. In another embodiment of formula (II), A is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl.

In one embodiment of formula (II), A is optionally substituted with —(R¹)ₙ, wherein n is 0, 1, 2, or 3. In one embodiment of formula (II), R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁵, SR⁵, C(O)R⁵, C(O)NR⁶R⁷, C(O)OR⁵, OC(O)R⁵, OC(O)NR⁶R⁷, NR⁶R⁷, NR⁶C(O)R⁵, S(O)R⁵, S(O)NR⁶R⁷, S(O)₂R⁵, NR⁶S(O)₂R⁵, and S(O)₂NR⁶R⁷; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂NR^{a}, and S(O)₂NR^{b}R^{c}.

In another embodiment of formula (II), A is phenyl, n is 2, and R¹, at each occurrence, is halo.

In one embodiment of formula (II), B is phenyl, pyridinyl, pyrimidinyl, pyrazinyl, pyridazinyl, or pyrazolinyl. In another embodiment of formula (II), B is phenyl.

In one embodiment of formula (II), B is

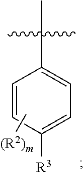

wherein R², R³, and m are as defined above. In another embodiment of formula (II), m is 0.

In another embodiment of formula (II), m is 1, and R², at each occurrence, is independently selected from the group consisting of halo, CN, OH, C₁₋₄ alkyl, C₁₋₄-haloalkyl, C₁₋₄ alkoxy, C₁₋₄ haloalkoxy, C₁₋₄-thioalkoxy, amino, C₁₋₄ alkylamino, and C₁₋₄ dialkylamino. In yet another embodiment of formula (II), m is 1 and R² is selected from the group consisting of halo, and C₁₋₄ alkoxy. In another embodiment of formula (II), R³ is selected from the group consisting aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁸, C(O)R⁸, C(O)NR⁹R¹⁰, C(O)OR⁸, OC(O)R⁸, OC(O)NR⁹R¹⁰, NR⁹R¹⁰, NR⁹C(O)R⁸, S(O)R⁸, S(O)NR⁹R¹⁰, S(O)₂R⁸, NR⁹S(O)₂R⁸, and S(O)₂NR⁹R¹⁰, wherein the C₃₋₈ cycloalkyl, aryl, heterocycloalkyl, and heteroaryl, alone or part of another moiety, are optionally substituted with one, two, or three R¹¹, wherein R¹¹ is defined above. In yet another embodiment of formula (II), B is phenyl, and R³ is heterocycloalkyl. In yet another embodiment of formula (II), R³ is heterocycloalkyl. In yet another embodiment of formula (II), R³ is heterocycloalkyl, which is optionally substituted with one R¹¹, and R¹¹ is selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{d}, R^{e}, and R^{f} are as defined above. In yet another embodiment of formula (II), R³ is heterocycloalkyl, which is optionally substituted with one R¹¹, and R¹¹ is selected from the group consisting of C₁₋₄ alkyl and NR^{e}R^{f}. In yet another embodiment of formula (II), R³ is heterocycloalkyl, which is optionally substituted with one R¹¹, and R¹¹ is C₁₋₄ alkyl. In yet another embodiment of formula (II), R³ is heterocycloalkyl, which is optionally substituted with one R¹¹, and R¹¹ is NR^{e}R^{f}, wherein R^{e} and R^{f} are C₁-6 alkyl.

In one embodiment of formula (II), B is

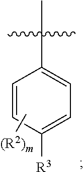

m is 0 or 1;
R² is halo or C₁₋₄ alkoxy;

### R³ is

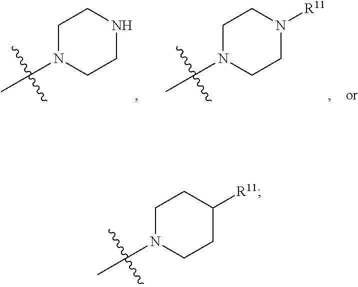

and
R¹¹ is halo, C₁₋₄ alkyl, C₁₋₄ haloalkyl, amino-C₁₋₄-alkyl-, C₁₋₄-alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C_{—}2 alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl.

In another embodiment of formula (II), B is

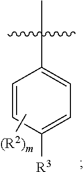

m is 0 or 1;
R² is halo, or C₁₋₄ alkoxy;

### R³ is

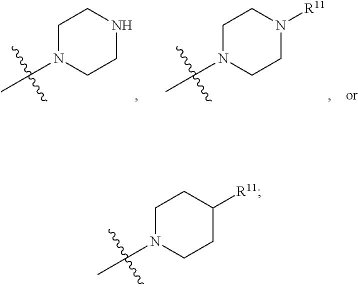

and
R¹¹ is C₁₋₄ alkyl, or NR^{e}R^{f}.

In another embodiment of formula (II), B is

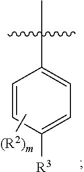

m is 0 or 1;
R² is halo, or C₁₋₄ alkoxy;

### R³ is

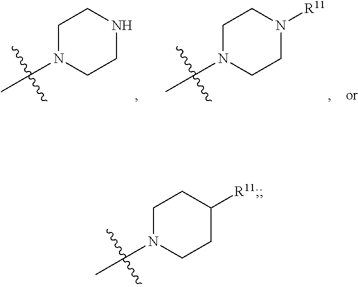

R¹⁰ is C₁₋₄ alkyl, or NR^{e}R^{f}; and
R^{e} and R^{f} are C₁₋₆ alkyl.

In one embodiment of formula (II), the present invention is directed; in part, to a class of compounds having a structure of Formula (IIb),

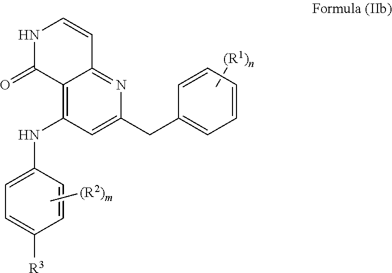

wherein R¹, R², R³, m, and n are as described in formula (I).

In one embodiment of formula (IIb), n is 0, 1, 2, or 3. In one embodiment of formula (IIb), R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁵, SR⁵, C(O)R⁵, C(O)NR⁶R⁷, C(O)OR⁵, OC(O)R⁵, OC(O)NR⁶R⁷, NR⁶R⁷, NR⁶C(O)R⁵, S(O)R⁵, S(O)NR⁶R⁷, S(O)₂R⁵, NR⁶S(O)₂R⁵, and S(O)₂NR⁶R⁷; wherein the C₃₋₈ cycloalkyl, aryl, heterocycloalkyl; and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c}.

In another embodiment of formula (IIb), n is 2, and R¹, at each occurrence, is halo. In another embodiment of formula (IIb), m is 0. In another embodiment of formula m is 1, and R², at each occurrence, is independently selected from the group consisting of halo, CN, OH, C₁₋₄ alkyl, C₁₋₄-haloalkyl, C₁₋₄ alkoxy, C₁₋₄ haloalkoxy, C₁₋₄-thioalkoxy, amino, C₁₋₄ alkylamino, and C₁₋₄ dialkylamino. In yet another embodiment of formula (IIb), m is 1 and R² is selected from the group consisting of halo, and C₁₋₄ alkoxy. In another embodiment of formula (IIb), R³ is selected from the group consisting aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁸, C(O)R⁸, C(O)NR⁹R¹⁰, C(O)OR⁸, OC(O)R⁸, OC(O)NR⁹R¹⁰, NR⁹R¹⁰, NR⁹C(O)R⁸, S(O)R⁸, S(O)NR⁹R¹⁰, S(O)₂R⁸, NR⁹S(O)₂R⁸, and S(O)₂NR⁹R¹⁰, wherein the C₃₋₈ cycloalkyl, aryl, heterocycloalkyl, and heteroaryl, alone or part of another moiety, are optionally substituted with one, two, or three R¹¹, wherein R¹¹ is defined above. In yet another embodiment of formula (IIb), R³ is heterocycloalkyl. In yet another embodiment of formula (IIb). R³ is heterocycloalkyl, which is optionally substituted with one R¹¹, and R¹¹ is selected from the group consisting of halo, C₁₋₄ alkyl, C₁₋₄ haloalkyl, amino-C₁₋₄-alkyl-, C₁₋₄ alkylamino-C₁₄ alkyl-, C₁₋₄ dialkylamino-C₁₋₄ hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{d}, R^{e}, and R^{f} are as defined above. In yet another embodiment of formula (IIb), R³ is heterocycloalkyl, which is optionally substituted with one R¹¹, and R¹¹ is selected from the group consisting of C₁₋₄ alkyl and NR^{e}R^{f}. In yet another embodiment of formula (IIb), R³ is heterocycloalkyl, which is optionally substituted with one R¹¹, and R¹¹ is C₁₋₄ alkyl. In yet another embodiment of formula (IIb), R³ is heterocycloalkyl, which is optionally substituted with one Rⁿ, and R¹¹ is NR^{e}R^{f}, wherein R^{e} and R^{f} are C₁₋₆ alkyl.

In one embodiment of formula (IIb),

m is 0 or 1;
R² is halo or C₁₋₄ alkoxy;

### R³ is

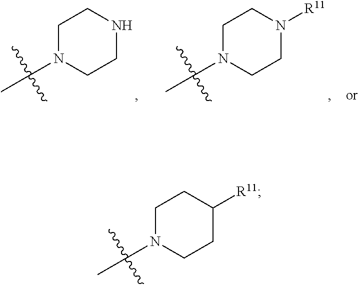

and
R¹¹ is halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ hydroxy-C₁₋₄-alkyl-, C₁₋₄ alkyl-C₁₋₄-alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl.

In another embodiment of formula (IIb),

m is 0 or 1;
R² is halo, or C₁₋₄ alkoxy;

### R³ is

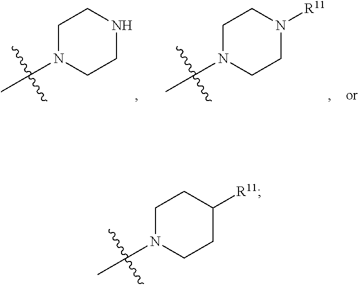

and
R¹¹ is C₁₋₄ alkyl, or NR^{e}R^{f}.

In another embodiment of formula (IIb),

m is 0 or 1;
R² is halo, or C₁₋₄ alkoxy;

### R³ is

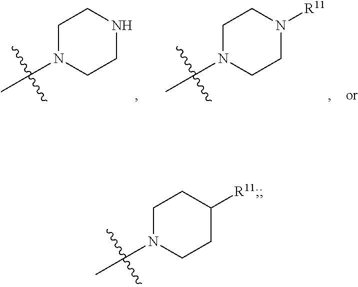

R¹¹ is C₁₋₄ alkyl, or NR^{e}R^{f}; and
R^{e} and R^{f} are C₁₋₆ alkyl.

### Embodiments of Formula (III)

In one embodiment, the present invention is directed, in part, to a class of compounds having a structure of Formula (III),

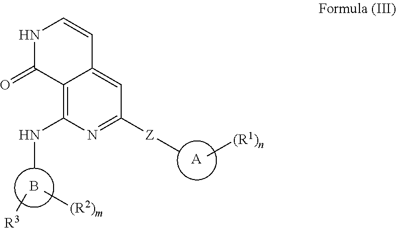

wherein R¹, R², R³, A, B, Z, m, and n are as described in formula (I).

In one embodiment of formula (III), Z is C₁₋₆ alkylene. In another embodiment of formula (III). Z is —CH₂—, —CH₂CH₂—, —CH₂CH₂CH₂—, or —CH₂CH₂CH₂CH₂—. In another embodiment of formula (III), Z is —CH(CH₃)—, —CH₂CH(CH₃)—, —CH(CH₃)CH₂—, —CH(CH₃)CH₂CH₂—, —CH₂CH(CH₃)CH₂—, —CH₂CH₂CH(CH₃)—, —C(CH₃)₂—, —CH₂C(CH₃)₂—, —C(CH₃)₂CH₂—, —CH₂CH₂C(CH₃)₂—, —CH₂C(CH₃)₂CH₂—, or —C(CH₃)₂CH₂CH₂—. In another embodiment of formula (III), Z is CH(CH₂CH₃)—, —CH₂CH(CH₂CH₃)—, —CH(CH₂CH₃)CH₂—, —CH(CH₂CH₃)CH₂CH₂—, —CH₂CH(CH₂CH₃)CH₂—, —CH₂CH₂CH(CH₂CH₃)—, —C(CH₂CH₃)₂—, —CH₂C(CH₂CH₃)₂—, —C(CH₂CH₃)₂CH₂—, —CH₂CH₂C(CH₂CH₃)₂—, —CH₂C(CH₂CH₃)₂CH₂—, or —C(CH₂CH₃)₂CH₂CH₂—. In yet another embodiment of formula (III), Z is —CH₂—, —CH₂CH₂—, —CH(CH₃)—, or —C(CH₃)₂—. In yet another embodiment of formula (III), Z is —CH₂—.

In one embodiment of formula (III), A is phenyl, naphthyl, indenyl or C₃₋₈ cycloalkyl. In yet another embodiment of formula (III), A is phenyl.

In another embodiment of formula (III), A is a 5-7 membered heterocycloalkyl or heterocycloalkenyl. In another embodiment of formula (III), A is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (III), A is dihydrofuranyl, dihydrothiophenyl, pyrrolinyl, imidazolinyl, pyrazolinyl, thiazolinyl, isothiazolinyl, dihydropyranyl, oxathiazinyl, oxadiazinyl, or oxazinyl.

In one embodiment of formula (III), A is a 5-7 membered heteroaryl. In another embodiment of formula (III), A is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl.

In one embodiment of formula (III), A is optionally substituted with —(R¹)ₙ, wherein n is 0, 1, 2, or 3. In one embodiment of formula (III), R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁵, SR⁵, C(O)R⁵, C(O)NR⁶R⁷, C(O)OR⁵, OC(O)R⁵, OC(O)NR⁶R⁷, NR⁶R⁷, NR⁶C(O)R⁵, S(O)R⁵, S(O)NR⁶R⁷, S(O)₂R⁵, NR⁶S(O)₂R⁵, and S(O)₂NR⁶R⁷; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c}.

In another embodiment of formula (III), A is phenyl, n is 2, and R¹, at each occurrence, is halo.

In one embodiment of formula (III), B is phenyl, pyridinyl, pyrimidinyl, pyrazinyl, pyridazinyl, or pyrazolinyl. In another embodiment of formula (III), B is phenyl.

In one embodiment of formula (III), B is

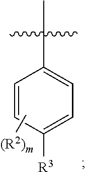

wherein R², R³, and in are as defined above. In another embodiment of formula (III), m is 0. In another embodiment of formula (III), m is 1, and R², at each occurrence, is independently selected from the group consisting of halo, CN, OH, C₁₋₄ alkyl, C₁₋₄-haloalkyl, C₁₋₄ alkoxy, C₁₋₄ haloalkoxy, C₁₋₄-thioalkoxy, amino, C₁₋₄ alkylamino, and C₁₋₄ dialkylamino. In yet another embodiment of formula (III), m is 1 and R² is selected from the group consisting of halo, and C₁₋₄ alkoxy. In another embodiment of formula (III), R³ is selected from the group consisting aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁸, C(O)R⁸, C(O)NR⁹R¹⁰, C(O)OR⁸, OC(O)R⁸, OC(O)NR⁹R¹⁰, NR⁹R¹⁰, NR⁹C(O)R⁸, S(O)R⁸, S(O)NR⁹R¹⁰, S(O)₂R⁸, NR⁹S(O)₂R⁸, and S(O)₂NR⁹R¹⁰, wherein the C₃₋₈ cycloalkyl, aryl, heterocycloalkyl, and heteroaryl, alone or part of another moiety, are optionally substituted with one, two, or three R¹¹, wherein R¹¹ is defined above. In yet another embodiment of formula (III), B is phenyl, and R³ is heterocycloalkyl. In yet another embodiment of formula (III), R³ is heterocycloalkyl. In yet another embodiment of formula (III), R³ is heterocycloalkyl, which is optionally substituted with one R¹¹, and R¹¹ is selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{d}, R^{e}, and R^{f} are as defined above. In yet another embodiment of formula (III), R³ is heterocycloalkyl, which is optionally substituted with one R¹¹, and R¹¹ is selected from the group consisting of C₁₋₄ alkyl and NR^{e}R^{f}. In yet another embodiment of formula (III), R³ is heterocycloalkyl, which is optionally substituted with one R¹¹, and R¹¹ is C₁₋₄ alkyl. In yet another embodiment of formula (III), R³ is heterocycloalkyl, which is optionally substituted with one and R¹ is NR^{e}R^{f}, wherein R^{e} and R^{f} are C₁₋₆ alkyl.

In one embodiment of formula (III), B is

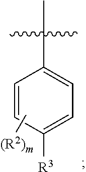

m is 0 or 1;
R² is halo or C₁₋₄ alkoxy;

### R³ is

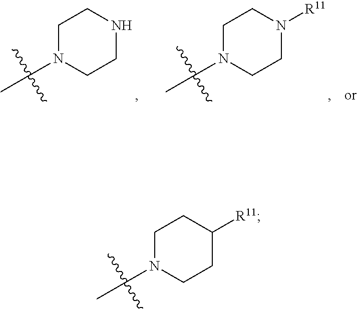

and
R¹¹ is halo, C₁₋₄ alkyl, C₁₋₄ haloalkyl, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl.

In another embodiment of formula (III), B is

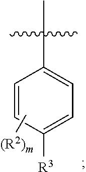

m is 0 or 1;
R² is halo, or C₁₋₄ alkoxy;

### R³ is

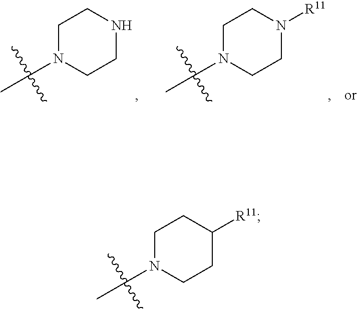

and
R¹⁰ is C₁₋₄ alkyl, or NR^{e}R^{f}.

In another embodiment of formula (III), B is

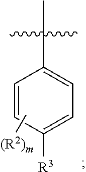

m is 0 or 1;
R² is halo, or C₁₋₄ alkoxy;

### R³ is

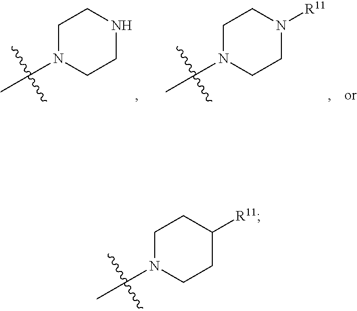

R¹¹ is C₁₋₄ alkyl, or NR^{e}R^{f}; and
R^{e} and R^{f} are C₁₋₆ alkyl.

In one embodiment of formula (III), the present invention is directed, in part, to a class of compounds having a structure of Formula (IIb),

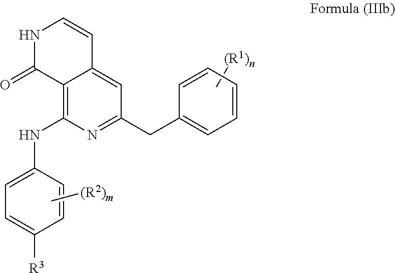

wherein R¹, R², R³, m, and n are as described in formula (I).

In one embodiment of formula (IIb), n is 0, 1, 2, or 3. In one embodiment of formula (IIb), R¹, at each occurrence, is independently selected from the group consisting of halo, CN, NO₂, C₁₋₆-haloalkyl, aryl, C₃₋₈ cycloalkyl, heteroaryl, heterocycloalkyl, OR⁵, SR⁶, C(O)R⁵, C(O)NR⁶R⁷, C(O)OR⁵, OC(O)R⁵, OC(O)NR⁶R⁷, NR⁶R⁷, NR⁶C(O)R⁵, S(O)R⁵, S(O)NR⁶R⁷, S(O)₂R⁵, NR⁶S(O)₂R⁵, and S(O)₂NR⁶R⁷; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c}.

In another embodiment of formula (IIIb), n is 2, and R¹, at each occurrence, is halo.

In another embodiment of formula (IIb), m is 0. In another embodiment of formula (IIb), m is 1, and R², at each occurrence, is independently selected from the group consisting of halo, CN, OH, C₁₋₄ alkyl, C₁₋₄-haloalkyl, C₁₋₄ alkoxy, C₁₋₄ haloalkoxy, C₁₋₄-thioalkoxy, amino, C₁₋₄ alkylamino, and C₁₋₄ dialkylamino. In yet another embodiment of formula (IIIb), m is 1 and R² is selected from the group consisting of halo, and C₁₋₄alkoxy. In another embodiment of formula (IIb), R³ is selected from the group consisting aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁸, C(O)R⁸, C(O)NR⁹R¹⁰, C(O)OR⁸, OC(O)R⁸: OC(O)NR⁹R¹⁰, NR⁹R¹⁰, NR⁹C(O)R⁸, S(O)R⁸, S(O)NR⁹R¹⁰, S(O)₂R⁸, NR⁹S(O)₂R⁸, and S(O)₂NR⁹R¹⁰, wherein the C₃₋₈ cycloalkyl, aryl, heterocycloalkyl, and heteroaryl, alone or part of another moiety, are optionally substituted with one, two, or three R¹¹, wherein R¹ is defined above. In yet another embodiment of formula (IIIb), R³ is heterocycloalkyl. In yet another embodiment of formula (IIIb), R³ is heterocycloalkyl, which is optionally substituted with one R¹¹, and R¹¹ is selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ hydroxy-C₁₋₄-alkyl-, C₁₋₄ alkyl-C₁₋₄alkoxy, aryl, C₃₋₈ cycloalkyl, heteroaryl, heterocycloalkyl, aryl-(C₁₋₂ alkyl)-, C₃₋₈ cycloalkyl-(C₁₋₂ heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{d}, R^{e}, and R^{f} are as defined above. In yet another embodiment of formula (IIIb), R³ is heterocycloalkyl, which is optionally substituted with one R¹¹, and R¹¹ is selected from the group consisting of C₁₋₄ alkyl and NR^{e}R^{f}. In yet another embodiment of formula (IIIb), R³ is heterocycloalkyl, which is optionally substituted with one R¹¹, and R¹¹ is C₁₋₄ alkyl. In yet another embodiment of formula (IIIb), R³ is heterocycloalkyl, which is optionally substituted with one R¹¹, and R¹¹ is NR^{e}R^{f}, wherein R^{e} and R^{f} are C₁₋₆ alkyl.

In one embodiment of formula (IIb),

m is 0 or 1;
R² is halo or C₁₋₄ alkoxy;

### R³ is

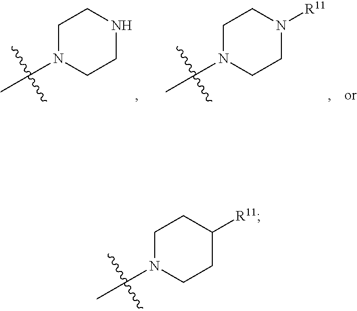

and
R¹¹ is halo, C₁₋₄ alkyl, C₁₋₄ haloalkyl, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl.

In another embodiment of formula (IIIb),

m is 0 or 1;
R² is halo, or C₁₋₄ alkoxy;

### R³ is

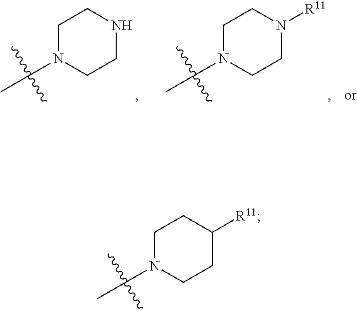

and
R^{I1} is C₁₋₄ alkyl, or NₑR^{f}.

In another embodiment of formula (IIIb),

m is 0 or 1;
R² is halo, or C₁₋₄ alkoxy;

### R³ is

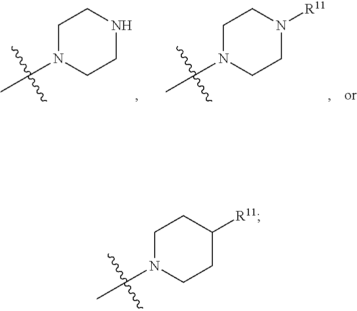

R¹¹ is C₁₋₄ alkyl, or NR^{e}R^{f}; and
R^{e} and R^{f} are C₁₋₆ alkyl.

### Embodiments of Formula (IV)

In one embodiment, the present invention is directed, in part, to a class of compounds having a structure of Formula (IV),

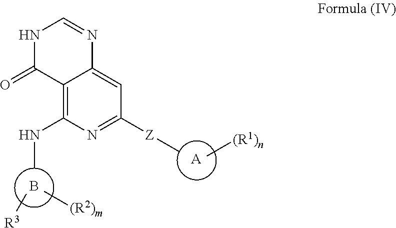

wherein R¹, R², R³, A, B, Z, m, and n are as described in formula (I).

In one embodiment of formula (IV), Z is C₁₋₆ alkylene. In another embodiment of formula (IV), Z is —CH₂—, —CH₂CH₂—, —CH₂CH₂CH₂—, or —CH₂CH₂CH₂CH₂—. In another embodiment of formula (IV), Z is —CH(CH₃)—, —CH₂CH(CH₃)—, —CH(CH₃)CH₂—, —CH(CH₃)CH₂CH₂—, —CH₂CH(CH₃)CH₂—, —CH₂CH₂CH(CH₃)—, —C(CH₃)₂—, —CH₂C(CH₃)₂—, —C(CH₃)₂CH₂—, —CH₂CH₂C(CH₃)₂—, —CH₂C(CH₃)₂CH₂—, or —C(CH₃)₂CH₂CH₂—. In another embodiment of formula (IV), Z is CH(CH₂CH₃)—, —CH₂CH(CH₂CH₃)—, —CH(CH₂CH₃)CH₂—, —CH(CH₂CH₃)CH₂CH₂—, —CH₂CH(CH₂CH₃)CH₂—, —CH₂CH₂CH(CH₂CH₃)—, —C(CH₂CH₃)₂—, —CH₂C(CH₂CH₃)₂—, —C(CH₂CH₃)₂CH₂—, —CH₂CH₂C(CH₂CH₃)₂—, —CH₂C(CH₂CH₃)₂CH₂—, or —C(CH₂CH₃)₂CH₂CH₂—. In yet another embodiment of formula (IV), Z is —CH₂—, —CH₂CH₂—, —CH(CH₃)—, or —C(CH₃)₂—. In yet another embodiment of formula (IV), Z is —CH₂—.

In one embodiment of formula (IV), A is phenyl, naphthyl, indenyl or C₃₋₈ cycloalkyl. In yet another embodiment of formula (IV), A is phenyl.

In another embodiment of formula (IV), A is a 5-7 membered heterocycloalkyl or heterocycloalkenyl. In another embodiment of formula (IV), A is pyrrolidinyl, tetrahydrofuryl, tetrahydrothienyl, imidazolidinyl, pyrazolidinyl, piperidinyl, tetrahydropyranyl, piperazinyl, dioxanyl, morpholinyl, 2-oxopyrrolidinyl, 2,5-dioxopyrrolidinyl, 2-oxopiperidinyl, 4-oxopiperidinyl, or 2,6-dioxopiperidinyl. In yet another embodiment of formula (IV), A is dihydrofuranyl, dihydrothiophenyl, pyrrolinyl, imidazolinyl, pyrazolinyl, thiazolinyl, isothiazolinyl, dihydropyranyl, oxathiazinyl, oxadiazinyl, or oxazinyl.

In one embodiment of formula (IV), A is a 5-7 membered heteroaryl. In another embodiment of formula (IV), A is pyridyl, pyrazyl, pyridinyl, pyrimidinyl, pyridazinyl, 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl, or isothiazolyl.

In one embodiment of formula (IV), A is optionally substituted with —(R¹)ₙ, wherein n is 0, 1, 2, or 3. In one embodiment of formula (IV), R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁵, SR⁵, C(O)R⁵, C(O)NR⁶R⁷, C(O)OR⁵, OC(O)R⁵, OC(O)NR⁶R⁷, NR⁶R⁷, NR⁶C(O)R⁵, S(O)R⁵, S(O)NR⁶R⁷, S(O)₂R⁵, NR⁶S(O)₂R⁵, and S(O)₂NR⁶R⁷; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c}.

In another embodiment of formula (IV), A is phenyl, n is 2, and R¹, at each occurrence, is halo.

In one embodiment of formula (IV), B is phenyl, pyridinyl, pyrimidinyl, pyrazinyl, pyridazinyl, or pyrazolinyl. In another embodiment of formula (IV), B is phenyl.

In one embodiment of formula (IV), B is

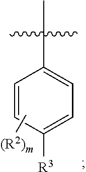

wherein R², R³, and m are as defined above. In another embodiment of formula (IV), m is 0. In another embodiment of formula (IV), m is 1, and R², at each occurrence, is independently selected from the group consisting of halo, CN, OH, C₁₋₄ alkyl, C₁₋₄-haloalkyl, C₁₋₄alkoxy, C₁₋₄ haloalkoxy, C₁₋₄-thioalkoxy, amino, C₁₋₄alkylamino, and C₁₋₄ dialkylamino. In yet another embodiment of formula (IV), m is 1 and R² is selected from the group consisting of halo, and C₁₋₄ alkoxy. In another embodiment of formula (IV), R³ is selected from the group consisting aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁸, C(O)R⁸, C(O)NR⁹R¹⁰, C(O)OR⁸, OC(O)R⁸, OC(O)NR⁹R¹⁰, NR⁹R¹⁰, NR⁹C(O)R⁸, S(O)R⁸, S(O)NR⁹R¹⁰, S(O)₂R⁸, NR⁹S(O)₂R⁸, and S(O)₂NR⁹R¹⁰, wherein the C₃₋₈ cycloalkyl, aryl, heterocycloalkyl, and heteroaryl, alone or part of another moiety, are optionally substituted with one, two, or three R¹¹, wherein R¹¹ is defined above. In yet another embodiment of formula (IV), B is phenyl, and R³ is heterocycloalkyl. In yet another embodiment of formula (IV), R³ is heterocycloalkyl. In yet another embodiment of formula (IV), R³ is heterocycloalkyl, which is optionally substituted with one R¹¹, and R¹¹ is selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{d}, R^{e}, and R^{f} are as defined above. In yet another embodiment of formula (IV), R³ is heterocycloalkyl, which is optionally substituted with one R¹¹, and R¹¹ is selected from the group consisting of C₁₋₄ alkyl and NR^{e}R^{f}. In yet another embodiment of formula (IV), R³ is heterocycloalkyl, which is optionally substituted with one R¹¹, and R¹¹ is C₁₋₄ alkyl. In yet another embodiment of formula (IV), R³ is heterocycloalkyl, which is optionally substituted with one R¹¹, and R¹¹ is NR^{e}R^{f}, wherein R^{e} and R^{f} are C₁₋₆ alkyl.

In one embodiment of formula (IV), B is

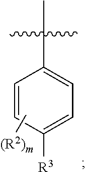

m is 0 or 1;
R² is halo or C₁₋₄alkoxy;

### R³ is

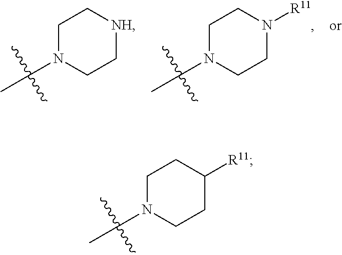

and
R¹¹ is halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl.

In another embodiment of formula (IV), B is

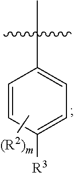

m is 0 or 1;
R² is halo, or C₁₋₄ alkoxy;

### R³ is

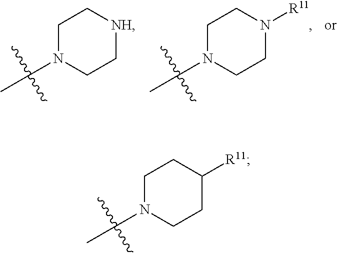

and
R¹¹ is C₁₋₄ alkyl, or NR^{e}R^{f}.

In another embodiment of formula (IV), B is

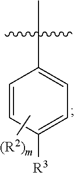

m is 0 or 1;
R² is halo, or C₁₋₄ alkoxy;

### R³ is

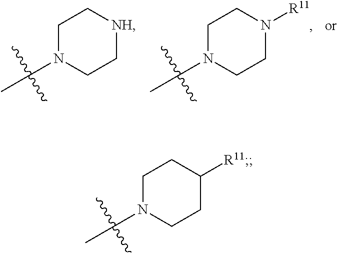

R¹¹ is C₁₋₄ alkyl, or NR^{e}R^{f}; and
R^{e} and R^{f} are C₁₋₆ alkyl.

In one embodiment of formula (IV), the present invention is directed, in part, to a class of compounds having a structure of Formula (IVb),

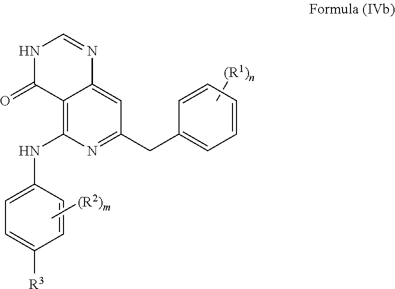

wherein R¹, R², R³, m, and n are as described in formula (I).

In one embodiment of formula (IVb), n is 0, 1, 2, or 3. In one embodiment of formula (IVb), R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁵, SR⁵, C(O)R⁵, C(O)NR⁶R⁷, C(O)OR⁵, OC(O)R⁵, OC(O)NR⁶R⁷, NR⁶R⁷, NR⁶C(O)R⁵, S(O)R⁵, S(O)NR⁶R⁷, S(O)₂R⁵, NR⁶S(O)₂R⁵, and S(O)₂NR⁶R⁷; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c}.

In another embodiment of formula (IVb), n is 2, and R¹, at each occurrence, is halo.

In another embodiment of formula (IVb), m is 0. In another embodiment of formula (IVb), m is 1, and R², at each occurrence, is independently selected from the group consisting of halo, CN, OH, C₁₋₄ alkyl, C₁₋₄-haloalkyl, C₁₋₄ alkoxy, C₁₋₄ haloalkyl, C₁₋₄-thioalkoxy, amino, C₁₋₄ alkylamino, and C₁₋₄ dialkylamino. In yet another embodiment of formula (IVb), m is 1 and R² is selected from the group consisting of halo, and C₁₋₄ alkoxy. In another embodiment of formula (IVb), R³ is selected from the group consisting aryl, C₃₋₈ cycloalkyl, heteroaryl, heterocycloalkyl, aryl-C₁₋₆-alkyl-, C₃₋₄ cycloalkyl-C₁₋₆-alkyl-, heteroaryl-C₁₋₆-alkyl-, heterocycloalkyl-C₁₋₆-alkyl-, OR⁸, C(O)R⁸, C(O)NR⁹R¹⁰, C(O)OR⁸, OC(O)R⁸, OC(O)NR⁹R¹⁰, NR⁹R¹⁰, NR⁹C(O)R⁸, S(O)R⁸, S(O)NR⁹R¹⁰, S(O)₂R⁸, NR⁹S(O)₂R⁸, and S(O)₂NR⁹R¹⁰, wherein the C₃₋₄ cycloalkyl, aryl, heterocycloalkyl, and heteroaryl, alone or part of another moiety, are optionally substituted with one, two, or three R¹¹, wherein R¹¹ is defined above. In yet another embodiment of formula (IVb), R³ is heterocycloalkyl. In yet another embodiment of formula (IVb), R³ is heterocycloalkyl, which is optionally substituted with one R¹¹, and R¹¹ is selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-. C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl; and wherein R^{d}, R^{e}, and R^{f} are as defined above. In yet another embodiment of formula (IVb), R³ is heterocycloalkyl, which is optionally substituted with one R¹¹, and R¹¹ is selected from the group consisting of C₁₋₄ alkyl and NR^{e}R^{f}. In yet another embodiment of formula (IVb), R³ is heterocycloalkyl, which is optionally substituted with one R¹¹, and R¹¹ is C₁₋₄ alkyl. In yet another embodiment of formula (IVb), R³ is heterocycloalkyl, which is optionally substituted with one R¹¹, and R¹¹ is NR^{e}R^{f}, wherein R^{e} and R^{f} are C₁₋₆ alkyl.

In one embodiment of formula (IVb),

m is 0 or 1;
R² is halo or C₁₋₄alkoxy;

### R³ is

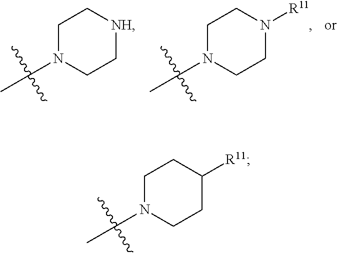

and
R¹¹ is halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl.

In another embodiment of formula (IVb),

m is 0 or 1;
R² is halo, or C₁₋₄ alkoxy;

### R³ is

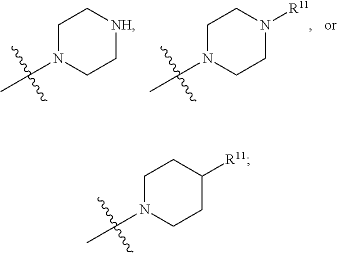

and
R¹¹ is C₁₋₄ alkyl, or NR^{e}R^{f}.

In another embodiment of formula (IVb),

m is 0 or 1;
R² is halo, or C₁₋₄ alkoxy;

### R³ is

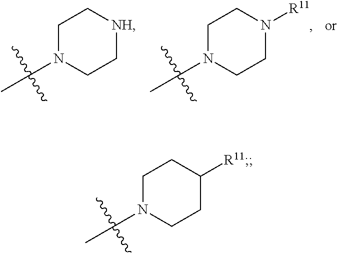

R¹¹ is C₁₋₄ alkyl, or NR^{e}R^{f}; and
Re and R^{f} are C₁₋₆ alkyl.

Specific embodiments contemplated as part of the invention include, but are not limited to, compounds of formula (I), for example:
2-(2,6-dichlorobenzyl)-4-{[2-methoxy-4-(4-methylpiperazin-1-yl)phenyl]amino}-1,6-naphthyridin-5(6H)-one;
2-(2,6-dichlorobenzyl)-4-{[2-methoxy-4-(piperazin-1-yl)phenyl]amino}-1,6-naphthyridin-5(6H)-one;
2-(2,6-dichlorobenzyl)-4-{[4-(piperazin-1-yl)phenyl]amino}-1,6-naphthyridin-5 (6H)-one;
6-(2,6-dichlorobenzyl)-8-{[2-methoxy-4-(4-methylpiperazin-1-yl)phenyl]amino}-2,7-naphthyridin-1(2H)-one;
7-(2,6-dichlorobenzyl)-5-{[2-methoxy-4-(4-methylpiperazin-1-yl)phenyl]amino}pyrido[4,3-d]pyrimidin-4(3H)-one;
7-(2,6-dichlorobenzyl)-5-{[2-methoxy-4-(piperazin-1-yl)phenyl]amino}pyrido[4,3-d]pyrimidin-4(3H)-one;
7-(2,6-dichlorobenzyl)-5-{[4-(piperazin-1-yl)phenyl]amino}pyrido[4,3-d]pyrimidin-4(3H)-one;
6-(2,6-dichlorobenzyl)-8-{[2-methoxy-4-(piperazin-1-yl)phenyl]amino}-2,7-naphthyridin-1 (2H)-one;
6-(2,6-dichlorobenzyl)-8-{[4-(piperazin-1-yl)phenyl]amino}-2,7-naphthyridin-1(2H)-one;
6-(2,6-dichlorobenzyl)-8-({4-[4-(dimethylamino)piperidin-1-yl]-2-methoxyphenyl}amino)-2,7-naphthyridin-1(2H)-one;
7-(2,6-dichlorobenzyl)-5-{[2-methoxy-4-(piperazin-1-yl)phenyl]amino}pyrido[2,3-d]pyrimidin-4(3H)-one;
7-(2,6-dichlorobenzyl)-5-{[2-methoxy-4-(piperazin-1-yl)phenyl]amino}pyrido[2,3-d]pyrimidin-4(3H)-one;
-(2,6-dichlorobenzyl)-5-{[2-methoxy-4-(piperazin-1-yl)phenyl]amino}pyrimido[4,5-d]pyrimidin-4(3H)-one;
2-(2,6-dichlorobenzyl)-4-{[3-fluoro-4-(piperazin-1-yl)phenyl]amino}-1,6-naphthyridin-5 (6H)-one;
2-(2,6-dichlorobenzyl)-4-{[2-methoxy-4-(piperazin-1-yl)phenyl]amino}pyrido[4,3-d]pyrimidin-5(6H)-one;
2-(2,6-dichlorobenzyl)-4-{[4-(piperazin-1-yl)phenyl]amino}pyrido[4,3-d]pyrimidin-5(6H)-one;
6-(2-chloro-6-fluorobenzyl)-8-{[4-(piperazin-1-yl)phenyl]amino}-2,7-naphthyridin-1 (2H)-one;

6-(2,6-dichlorobenzyl)-8-({2-methoxy-4-[(4-methylpiperazin-1-yl)carbonyl]phenyl}amino)-2,7-naphthyridin-1(2H)-one;
4-{[2-(2,6-dichlorobenzyl)-5-oxo-5,6-dihydro-1,6-naphthyridin-4-yl]amino}-3-methoxy-N-[3-(morpholin-4-yl)propyl]benzamide;
4-{[3-(2,6-dichlorobenzyl)-8-oxo-7,8-dihydro-2,7-naphthyridin-1-yl]amino}-3-methoxy-N-[3-(morpholin-4-yl)propyl]benzamide;
2-(2,6-dichlorobenzyl)-4-({2-methoxy-4-[(4-methylpiperazin-1-yl)carbonyl]phenyl}amino)-1,6-naphthyridin-5(6H)-one;
4-{[2-(2,6-dichlorobenzyl)-5-oxo-5,6-dihydro-1,6-naphthyridin-4-yl]amino}-3-methoxy-N-[2-(pyrrolidin-1-yl)ethyl]benzamide;
4-{[3-(2,6-dichlorobenzyl)-8-oxo-7,8-dihydro-2,7-naphthyridin-1-yl]amino}-3-methoxy-N-[2-(pyrrolidin-1-yl)ethyl]benzamide;
4-{[2-(2,6-dichlorobenzyl)-5-oxo-5,6-dihydro-1,6-naphthyridin-4-yl]amino}-N-[2-(dimethylamino)ethyl]-3-methoxybenzamide;
4-{[2-(2,6-dichlorobenzyl)-5-oxo-5,6-dihydro-1,6-naphthyridin-4-yl]amino}-3-methoxy-N-[2-(piperidin-1-yl)ethyl]benzamide;
4-{[2-(2,6-dichlorobenzyl)-5-oxo-5,6-dihydro-1,6-naphthyridin-4-yl]amino}-3-methoxy-N-[2-(4-methylpiperazin-1-yl)ethyl]benzamide;
4-{[3-(2,6-dichlorobenzyl)-8-oxo-7,8-dihydro-2,7-naphthyridin-1-yl]amino}-3-methoxy-N-[2-(4-methylpiperazin-1-yl)ethyl]benzamide;

4-{[3-(2,6-dichlorobenzyl)-8-oxo-7,8-dihydro-2,7-naphthyridin-1-yl]amino}-3-methoxy-N-[2-(piperidin-1-yl)ethyl]benzamide;
4-{[3-(2,6-dichlorobenzyl)-8-oxo-7,8-dihydro-2,7-naphthyridin-1-yl]amino}-N-[4-(dimethylamino)butyl]-3-methoxybenzamide;
2-(2,6-dichlorobenzyl)-4-{[2-methoxy-4-(piperazin-1-ylcarbonyl)phenyl]amino}-1,6-naphthyridin-5(6H)-one;
4-{[2-(2,6-dichlorobenzyl)-5-oxo-5,6-dihydro-1,6-naphthyridin-4-yl]amino}-N-[4-(dimethylamino)butyl]-3-methoxybenzamide; and
2-(2-chlorobenzyl)-4-{[2-methoxy-4-(piperazin-1-yl)phenyl]amino}-1,6-naphthyridin-5(6H)-one.

Compounds of this invention may contain asymmetrically substituted carbon atoms in the R or S configuration, wherein the terms “R” and “S” are as defined in Pure Appl. Chem. (1976) 45, 13-10. Compounds having asymmetrically substituted carbon atoms with equal amounts of R and S configurations are racemic at those atoms. Atoms having excess of one configuration over the other are assigned the configuration in excess, preferably an excess of about 85%-90%, more preferably an excess of about 95%-99%, and still more preferably an excess greater than about 99%. Accordingly, this invention is meant to embrace racemic mixtures and relative and absolute diastereoisomers of the compounds thereof.

Compounds of this invention may also contain carbon-carbon double bonds or carbon-nitrogen double bonds in the E or Z configuration, wherein the term “E” represents higher order substituents on opposite sides of the carbon-carbon or carbon-nitrogen double bond and the term “Z” represents higher order substituents on the same side of the carbon-carbon or carbon-nitrogen double bond as determined by the Cahn-Ingold-Prelog Priority Rules. The compounds of this invention may also exist as a mixture of “E” and “Z” isomers.

Additional geometric isomers may exist in the present compounds. For example, the invention contemplates the various geometric isomers and mixtures thereof resulting from the disposition of substituents around a cycloalkyl group or a heterocycle group. Substituent: around a cycloalkyl or a heterocycle are designated as being of cis or trans configuration.

Compounds of this invention may also exist as tautomers or equilibrium mixtures thereof wherein a proton of a compound shifts from one atom to another. Examples of tautomers include, but are not limited to, keto-enol, phenol-keto, oxime-nitroso, nitro-aci, imine-enamine and the like. Tautomeric forms are intended to be encompassed by the scope of this invention, even though only one tautomeric form may be depicted.

This invention also is directed, in part, to all salts of the compounds of formula (I). A salt of a compound may be advantageous due to one or more of the salt's properties, such as, for example, enhanced pharmaceutical stability in differing temperatures and humidities, or a desirable solubility in water or other solvents. Where a salt is intended to be administered to a patient (as opposed to, for example, being in use in an in vitro context), the salt preferably is pharmaceutically acceptable and/or physiologically compatible. The term “pharmaceutically acceptable” is used adjectivally in this patent application to mean that the modified noun is appropriate for use as a pharmaceutical product or as a part of a pharmaceutical product. Pharmaceutically acceptable salts include salts commonly used to form alkali metal salts and to form addition salts of free acids or free bases. In general, these salts typically may be prepared by conventional means by reacting, for example, the appropriate acid or base with a compound of the invention.

Pharmaceutically acceptable acid addition salts of the compounds of formula (I) can be prepared from an inorganic or organic acid. Examples of often suitable inorganic acids include hydrochloric, hydrobromic, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Specific examples of often suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), ethanesulfonate, benzenesulfonate, pantothenate, 2-hydroxy ethanesulfonate, sulfanilate, cyclohexylaminosulfonate, algenic acid, beta-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, heptanoate, hexanoate, nicotinate, oxalate, palmoate, pectinate, 2-naphthalesulfonate, 3-phenylpropionate, picrate, pivalate, thiocyanate, tosylate, and undecanoate.

Pharmaceutically acceptable base addition salts of the compounds of formula (I) include, for example, metallic salts and organic salts. Preferred metallic salts include alkali metal (group Ia) salts, alkaline earth metal (group IIa) salts, and other physiologically acceptable metal salts. Such salts may be made from aluminum, calcium, lithium, magnesium, potassium, sodium, and zinc. Preferred organic salts can be made from amines, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

Compounds of formula (I) (and salts thereof) with any level of purity (including pure and substantially pure) are within the scope of Applicants' invention. The term “substantially pure” in reference to a compound/salt/isomer, means that the preparation/composition containing the compound/salt/isomer contains more than about 85% by weight of the compound/salt/isomer, preferably more than about 90% by weight of the compound/salt/isomer, preferably more than about 95% by weight of the compound/salt/isomer, preferably more than about 97% by weight of the compound/salt/isomer, and preferably more than about 99% by weight of the compound/salt/isomer.

### Preparation of Compounds

Compounds of this invention may be made by synthetic chemical processes, examples of which are shown herein. It is meant to be understood that the order of the steps in the processes may be varied, that reagents, solvents and reaction conditions may be substituted for those specifically mentioned, and that vulnerable moieties may be protected and deprotected, as necessary.

Protecting groups for C(O)OH moieties include, but are not limited to, acetoxymethyl, allyl, benzoylmethyl, benzyl, benzyloxymethyl, tert-butyl, tert-butyldiphenylsilyl, diphenylmethyl, cyclobutyl, cyclohexyl, cyclopentyl, cyclopropyl, diphenylmethylsilyl, ethyl, para-methoxybenzyl, methoxymethyl, methoxyethoxymethyl, methyl, methylthiomethyl, naphthyl, para-nitrobenzyl, phenyl, n-propyl, 2,2,2-trichloroethyl, triethylsilyl, 2-(trimethylsilyl)ethyl, 2-(trimethylsilyl)ethoxymethyl, triphenylmethyl and the like.

Protecting groups for C(O) and C(O)H moieties include, but are not limited to, 1,3-dioxylketal, diethylketal, dimethylketal, 1,3-dithianylketal, O-methyloxime, O-phenyloxime and the like.
Protecting groups for NH moieties include, but are not limited to, acetyl, alanyl, benzoyl, benzyl(phenylmethyl), benzylidene, benzyloxycarbonyl (Cbz), tert-butoxycarbonyl (Boc), 3,4-dimethoxybenzyloxycarbonyl, diphenylmethyl, diphenylphosphoryl, formyl, methanesulfonyl, para-methoxybenzyloxycarbonyl, phenylacetyl, phthaloyl, succinyl, trichloroethoxycarbonyl, triethylsilyl, trifluoroacetyl, trimethylsilyl, triphenylmethyl, triphenylsilyl, para-toluenesulfonyl and the like.

Protecting groups for OH and SH moieties include, but are not limited to, acetyl, allyl, allyloxycarbonyl, benzyloxycarbonyl (Cbz), benzoyl, benzyl, tert-butyl, tert-butyldimethylsilyl, tert-butyldiphenylsilyl, 3,4-dimethoxybenzyl, 3,4-dimethoxybenzyloxycarbonyl, 1,1-dimethyl-2-propenyl, diphenylmethyl, formyl, methanesulfonyl, methoxyacetyl, 4-methoxybenzyloxycarbonyl, para-methoxybenzyl, methoxycarbonyl, methyl, para-toluenesulfonyl, 2,2,2-trichloroethoxycarbonyl, 2,2,2-trichloroethyl, triethylsilyl, trifluoroacetyl, 2-(trimethylsilyl)ethoxycarbonyl, 2-trimethylsilylethyl, triphenylmethyl, 2-(triphenylphosphonio)ethoxycarbonyl and the like.

### Schemes

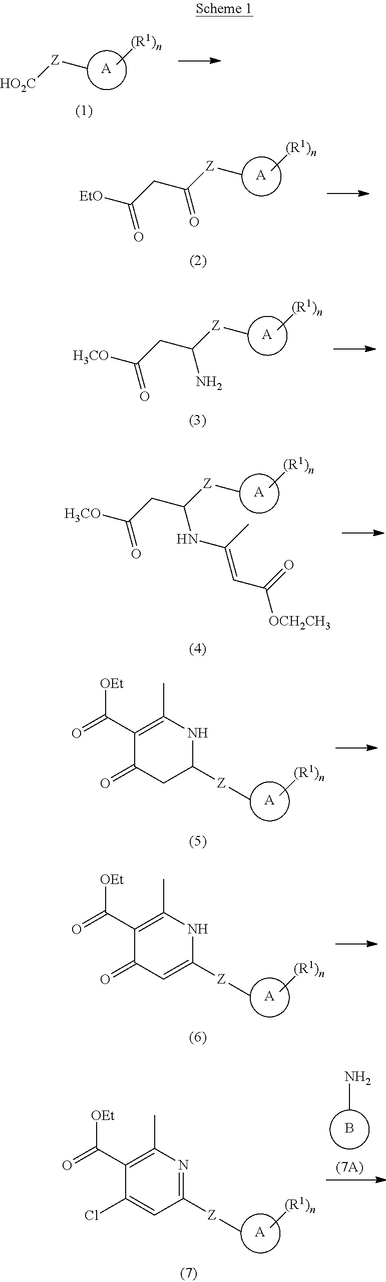

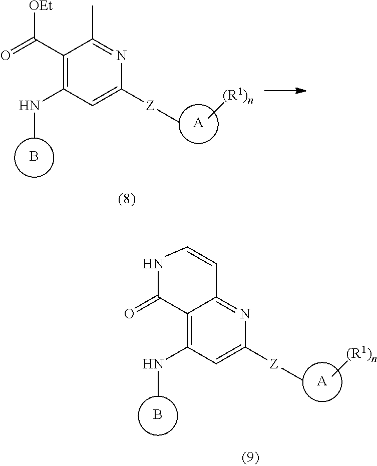

As shown in Scheme 1, compounds of formula (I), wherein A, Z, R¹ and n are as described herein, can be reacted at room temperature with N,N-carbonyldiimidazole in a solvent such as, but not limited to, dry tetrahydrofuran, followed by the addition magnesium chloride and ethyl potassium malonate at elevated temperature, to provide compounds of formula (2), wherein Et is CH₂CH₃. Compounds of formula (3) can be prepared from compounds of formula (2) by reacting the latter with ammonium acetate, magnesium sulfate, and sodium cyanoborohydride. The reaction is typically performed in a solvent such as, but not limited to, methanol at elevated temperatures. Compounds of formula (4) can be prepared by reacting compounds of formula (3) with acetic acid, ethyl acetoacetate, and magnesium sulfate. The reaction is typically performed at elevated temperature, in a solvent such as but not limited to toluene. Compounds of formula (4) can be reacted with a base such as, but not limited to, potassium t-butoxide at ambient temperature in a solvent such as but not limited to tetrahydrofuran, to provide compounds of formula (5), wherein Et is CH₂CH₃. Compounds of formula (6) can be prepared by reacting compounds of formula (5) with 2,3-dichloro-5,6-dicyano-1,4-benzoquinone. A solvent such as but not limited to tetrahydrofuran is typically employed. Compounds of formula (6) can be reacted with phosphorus oxychloride to provide compounds of formula (7). The reaction is typically performed at elevated temperature. Compounds of formula (8) can be prepared from compounds of formula (7) by reacting the latter with compounds of formula (7A), wherein B is as described herein, in the presence of p-toluenesulfonic acid. The reaction is typically performed at elevated temperature in a solvent such as but not limited to n-butanol. Compounds of formula (9), which are representative of the compounds of Formula (I), can be prepared by reacting compounds of formula (8) with sodium hydride, followed by 1,3,5-triazine at elevated temperature. The reaction is typically performed in a solvent such as but not limited to N,N-dimethylformamide.

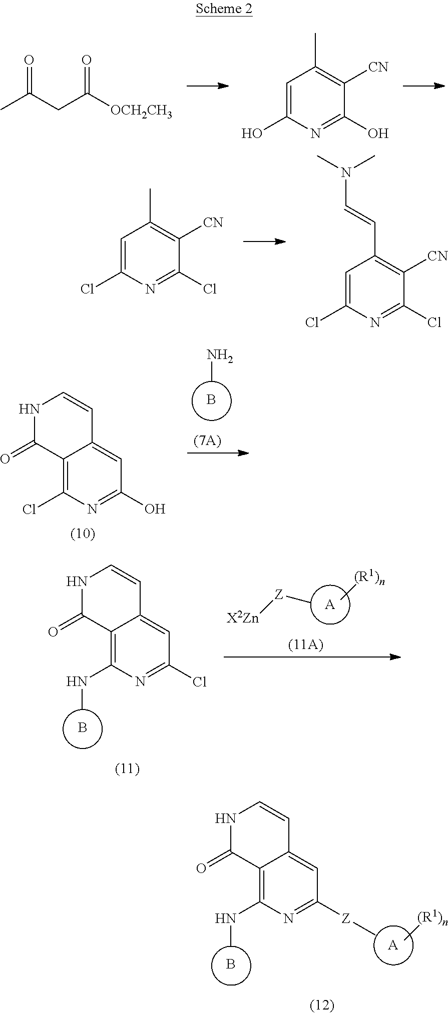

Potassium hydroxide can be added to a solution of 2-cyanoacetamide and ethyl 3-oxobutanoate in a solvent such as but not limited to methanol to provide 2,6-dihydroxy-4-methylnicotinonitrile, as shown in Scheme 2. The reaction is typically performed at elevated temperature. 2,6-Dihydroxy-4-methylnicotinonitrile and phosphorus oxychloride can heated in a sealed tube without an additional solvent to provide 2,6-dichloro-4-methylnicotinonitrile. N,N-Dimethylformamide dimethyl acetal can be added to a solution of 2,6-dichloro-4-methylnicotinonitrile in a solvent such as but not limited to N,N-dimethylformamide to provide (E)-2,6-dichloro-4-(2-(dimethylamino)vinyl)nicotinonitrile. The reaction is typically performed at an elevated temperature. Concentrated hydrochloric acid and (E)-2,6-dichloro-4-(2-(dimethylamino)vinyl)nicotinonitrile can be heated in a sealed tube to provide 6,8-dichloro-2,7-naphthyridin-1 (2H)-one (10). Compounds of formula (11) can be prepared from compounds of formula (10) by reacting the latter with compounds of formula (7A), wherein B is as described herein, in a solvent such as but not limited to N-methylpyrrolidone. The reaction is typically performed at elevated temperature and may be performed in a microwave oven. Compounds of formula (12), which are representative of the compounds of Formula (I), can be prepared by reacting compounds of formula (11) with an organozinc compound of formula (11A), wherein Z, A, R¹, and n are as described herein and X² is a halide. The reaction typically involves the use of heat and a nickel or palladium catalyst such as but not limited to bis(triphenylphosphine)palladium(II) dichloride in a solvent such as but not limited to N-methylpyrrolidone, tetrahydrofuran, or mixtures thereof.

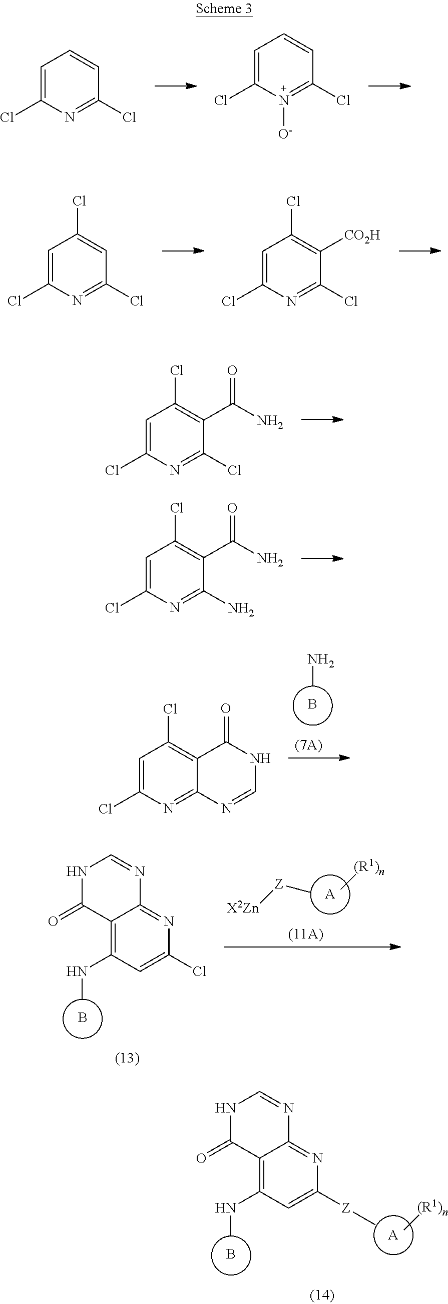

As shown in Scheme 3, 2,6-dichloropyridine 1-oxide can be prepared by reacting a solution of 2,6-dichloropyridine, 30% hydrogen peroxide, and an acid such as but not limited to trifluoroacetic acid at elevated temperature. 2,6-Dichloropyridine 1-oxide can be reacted with phosphorus oxychloride at elevated temperature to provide 2,4,6-trichloropyridine. Carboxylation of 2,4,6-trichloropyridine to provide 2,4,6-trichloronicotinic acid can be performed by adding solid carbon dioxide (dry ice) to a solution of 2,4,6-trichloropyridine and diisopropylamine treated with n-butyl lithium. The n-butyl lithium is typically added at low temperature to a mixture of 2,4,6-trichloropyridine and diisopropylamine in a solvent such as but not limited to tetrathydrofuran, before adding the carbon dioxide gas and warming to room temperature. 2,4,6-Trichloronicotinic acid can be treated at ambient temperature with oxalyl chloride in a solvent such as but not limited to dichloromethane, N,N-dimethylformamide, or mixtures thereof. Ammonia gas can be bubbled through a solution of the crude acid chloride in a solvent such as but not limited to tetrahydrofuran to provide 2,4,6-trichloronicotinamide. 2-Amino-4,6-dichloronicotinamide can be prepared by reacting 2,4,6-trichloronicotinamide with ammonia. The reaction is typically performed at elevated temperature in a solvent such as but not limited to 1,4-dioxane. 2-Amino-4,6-dichloronicotinamide can be reacted with triethyl orthoformate at elevated temperature to provide 5,7-dichloropyrido[2,3-d]pyrimidin-4(3H)-one. 5,7-Dichloropyrido[2,3-d]pyrimidin-4(31-1)-one can be reacted with a compound of formula (7A), wherein B is as described herein, in the presence of a base such as but not limited to N,N-diisopropylethylamine or triethylamine, to provide compounds of formula (13). The reaction is typically performed at elevated temperature in a solvent such as but not limited to 1,4-dioxane. Compounds of formula (14), which are representative of the compounds of Formula (1), can be prepared by reacting compounds of formula (13) with an organic compound of formula (11A), wherein Z, A, R¹, and n are as described herein and X² is a halide. The reaction typically involves the use of heat, and a nickel or palladium catalyst such as but not limited to tetrakis(triphenylphosphine)palladium in a solvent such as but not limited to N-methylpyrrolidone, tetrahydrofuran, or mixtures thereof

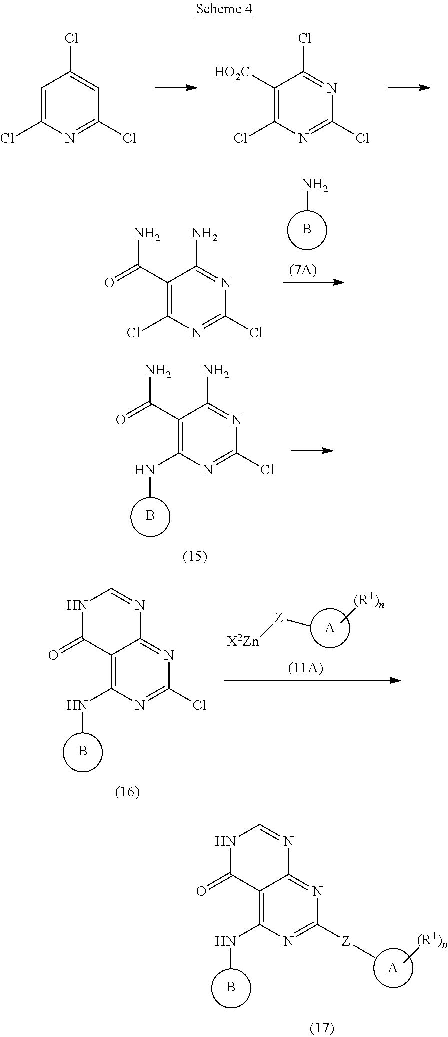

Carboxylation of 2,4,6-trichloropyrimidine to provide 2,4,6-trichloropyrimidine-5-carboxylic acid can be performed by adding solid carbon dioxide (dry ice) to a solution of 2,4,6-trichloropyridine and diisopropylamine treated with n-butyl lithium. The n-butyl lithium is typically added at low temperature to a mixture of 2,4,6-trichloropyridine and diisopropylamine in a solvent such as but not limited to tetrahydrofuran, before adding the carbon dioxide gas and warming to room temperature. 4-Amino-2,6-dichloropyrimidine-5-carboxamide can be prepared from 2,4,6-trichloropyrimidine-5-carboxylic acid by reacting the latter first with oxalyl chloride at low temperature in a solvent such as but not limited to dichloromethane, N,N-dimethylformamide, or mixtures thereof. The resulting crude acid chloride can be reacted with ammonium hydroxide at low temperature in a solvent such as but not limited to tetrahydrofuran to provide 4-amino-2,6-dichloropyrimidine-5-carboxamide. 4-Amino-2,6-dichloropyrimidine-5-carboxamide can be reacted with a compound of formula (7A), wherein B is as described herein, in the presence of a base such as but not limited to N,N-diisopropylethylamine or triethylamine, to provide compounds of formula (15). The reaction is typically performed at elevated temperature in a solvent such as but not limited to 1,4-dioxane. Triethyl orthoformate can be reacted with compounds of formula (15) to provide compounds of formula (16). The reaction typically involves the use of heat and may employ a solvent such as but not limited to N,N-dimethylformamide. Compounds of formula (17), which are representative of the compounds of Formula (I), can be prepared by reacting compounds of formula (16) with an organozinc compound of formula (11A), wherein Z, A, R¹, and n are as described herein and X² is a halide. The reaction typically involves the use of heat, and a nickel or palladium catalyst such as but not limited to bis(triphenylphosphine)palladium(II) dichloride in a solvent such as but not limited to N-methylpyrrolidone, tetrahydrofuran, or mixtures thereof.

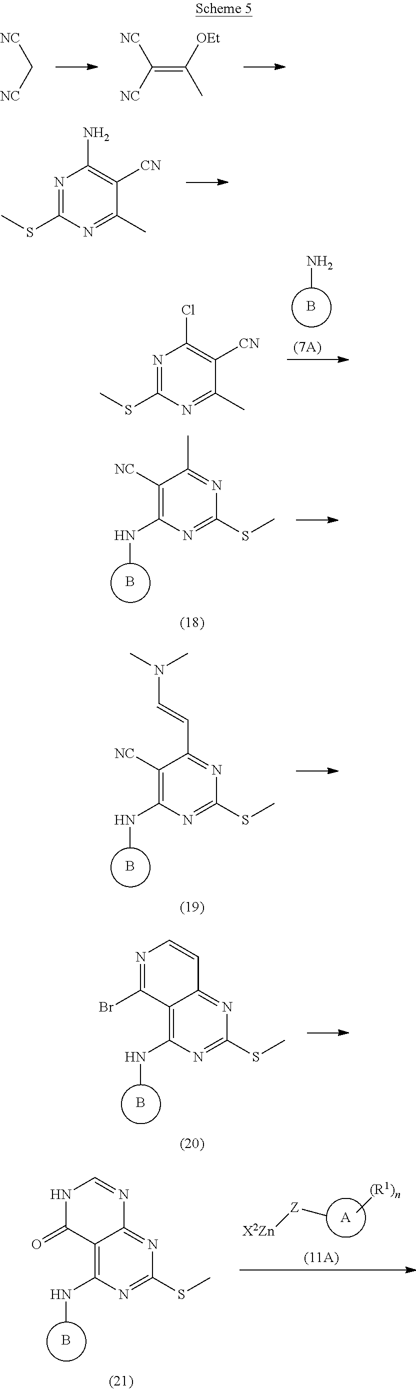

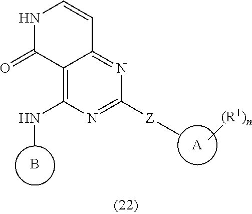

Malononitrile can be reacted with triethyl orthoacetate in glacial acetic acid to provide 2-(1-ethoxyethylidene)malononitrile. The reaction is typically performed at elevated temperature. 4-Amino-6-methyl-2-(methylthio)pyrimidine-5-carbonitrile can be prepared by reacting 2-(1-ethoxyethylidene)malononitrile with S-methlylisothiourea hemisulfate salt in the presence of sodium methanolate. The addition is typically performed in a solvent such as but not limited to methanol at reduced temperature before warming to ambient temperature. A mixture of anhydrous copper (II) chloride and tert-butylnitrite can be reacted with 4-amino-6-methyl-2-(methylthio)pyrimidine-5-carbonitrile to provide 4-chloro-6-methyl-2-(methylthio)pyrimidine-5-carbonitrile. The reaction is typically performed at elevated temperature in a solvent such as but not limited to acetonitrile. 4-Chloro-6-methyl-2-(methylthio)pyrimidine-5-carbonitrile can be reacted with a compound of (7A), wherein B is as described herein, in the presence of a base such as but not limited to N,N-diisopropylethylamine or triethylamine, to provide compounds of formula (18). The reaction is typically performed at elevated temperature in a solvent such as but not limited to N,N-dimethylformamide or 1,4-dioxane. Compounds of formula (18) can be reacted with N,N-dimethylformamide dimethyl acetal to provide compounds of formula (19). The reaction is typically performed at elevated temperature in a solvent such as but not limited to N,N-dimethylformamide. Compounds of formula (19) can be reacted with hydrobromic acid in glacial acetic acid to provide compounds of formula (20). The reaction is typically performed at ambient temperature. Compounds of formula (20) can be treated a mixture of acetic acid and aqueous hydrochloric acid to provide compounds of formula (21). The reaction is typically performed at elevated temperature. Compounds of formula (22), which are representative of the compounds of Formula (I), can be prepared by reacting compounds of formula (21) with an organozinc compound of formula (11A), wherein Z, A, R¹, and n are as described herein and X² is a halide. The reaction typically involves the use of heat, and a nickel or palladium catalyst such as but not limited to tris(dibenzylideneacetone)dipalladium or bis(triphenylphosphine)palladium(II) dichloride in a solvent such as but not limited to N-methylpyrrolidone, tetrahydrofuran, or mixtures thereof. Additionally, the reaction may be performed in a microwave oven.

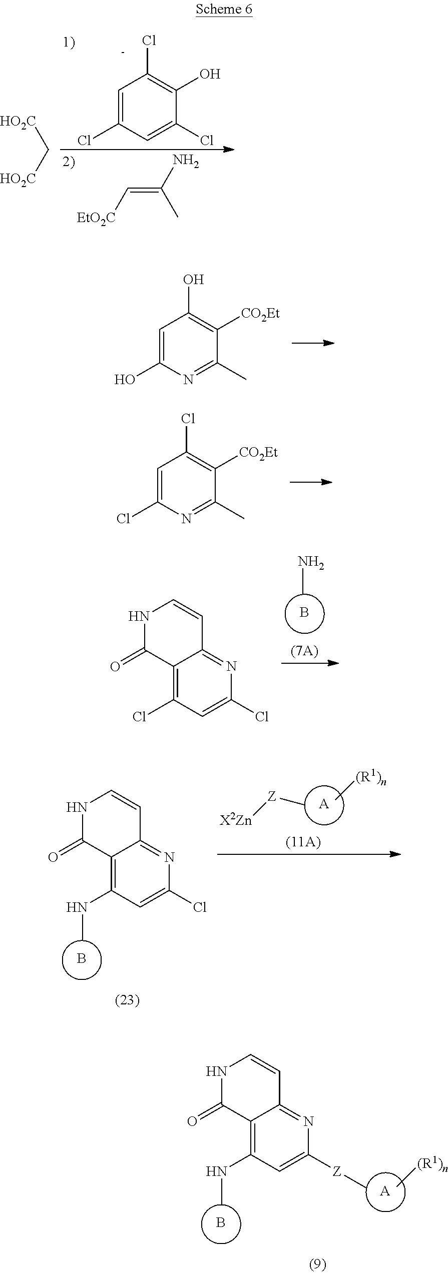

As shown in Scheme 6, ethyl 4,6-dihydroxy-2-methylnicotinate can be prepared by reacting 2,4,6-trichlorophenol, malonic acid and phosphorus oxychloride, followed by work up and reaction with ethyl 3-aminocrotonate. The first step is typically performed at elevated temperature. The second step is typically performed in a solvent such as but not limited to bromobenzene at an elevated temperature. Reaction of ethyl 4,6-dihydroxy-2-methylnicotinate with phosphorus oxychloride will provide ethyl 4,6-dichloro-2-methylnicotinate. The reaction is typically performed at elevated temperature. 2,4-Dichloro-1,6-naphthyridin-5(6H)-one can be prepared by reacting provide ethyl 4,6-dichloro-2-methylnicotinate will sodium hydride followed by triazine. The reaction is typically performed at ambient temperature in a solvent such as but not limited to N,N-dimethylformamide, toluene, or mixtures thereof. 2,4-Dichloro-1,6-naphthyridin-5(6H)-one can be reacted with a compound of (7A), wherein B is as described herein, in the presence of a base such as but not limited to N,N—N,N-diisopropylethylamine or triethylamine, to provide compounds of formula (23). The reaction is typically performed at elevated temperature in a solvent such as but not limited to N,N-dimethylformamide or 1,4-dioxane. Compounds of formula (9), which are representative of the compounds of Formula (I), can be prepared by reacting compounds of formula (23) with an organozinc compound of formula (11A), wherein Z, A, R¹, and n are as described herein and X² is a halide. The reaction typically involves the use of heat, and a nickel or palladium catalyst such as but not limited to tetrakis(triphenylphosphine)palladium, tris(dibenzylideneacetone)dipalladium or bis(triphenylphosphine)palladium(II) dichloride in a solvent such as but not limited to N-methylpyrrolidone, tetrahydrofuran, or mixtures thereof. Additionally, the reaction may be performed in a microwave oven.

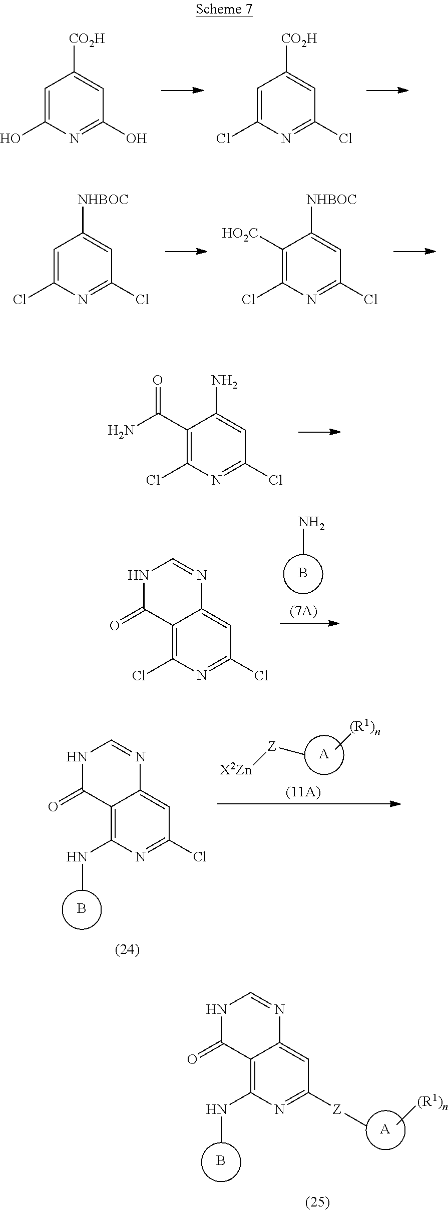

As shown in Scheme 7, 2,6-dihydroxypyridine-4-carboxylic acid and phosphoryl trichloride can be heated in a sealed tube to provide 2,6-dichloropyridine-4-carboxylic acid. Diphenylphosphoryl azide and a base such as but not limited to N,N—N,N-diisopropylethylamine in tert-butanol can be added to 2,6-dichloropyridine-4-carboxylic acid to provide tert-butyl 2,6-dichloropyridin-4-ylcarbamate. The reaction typically requires the use of heat. Carboxylation of tert-butyl 2,6-dichloropyridin-4-ylcarbamate to provide 4-(tert-butoxycarbonylamino)-2,6-dichloronicotinic acid can be performed by bubbling dry carbon dioxide gas through a solution of tert-butyl 2,6-dichloropyridin-4-ylcarbamate and N,N,N′,N′-tetramethylethylenediamine treated with n-butyl lithium. The n-butyl lithium is typically added at low temperature to a mixture of tert-butyl 2,6-dichloropyridin-4-ylcarbamate and N,N,N′,N′-tetramethylethylenediamine in a solvent such as but not limited to tetrathydrofuran, before adding the carbon dioxide and warming to room temperature. A solution of 4-(tert-Butoxycarbonylamino)-2,6-dichloronicotinic acid and 1,1′-carbonyldiimidazole in solvent such as but not limited to N,N-dimethylformamide can be stirred at elevated temperature before the addition of ammonia gas at reduced temperature to provide 4-amino-2,6-dichloropyridine-3-carboxamide. Triethyl orthoformate can be reacted with 4-amino-2,6-dichloropyridine-3-carboxamide to provide 5,7-dichloropyrido[4,3-d]pyrimidin-4(3H)-one. The reaction typically involves the use of heat and a solvent such as but not limited to N,N-dimethylformamide. 5,7-Dichloropyrido[4,3-d]pyrimidin-4(3H)-one can be reacted with a compound of formula (7A), wherein B is as described herein, in the presence of a base such as but not limited to triethylamine, to provide compounds of formula (24). The reaction is typically performed at elevated temperature in a solvent such as but not limited to 1,4-dioxane. Compounds of formula (25), which are representative of compounds of Formula (I), can be prepared by reacting compounds of formula (24) with an organozinc compound of formula (11A), wherein Z, A, R¹, and n are as described herein and X² is a halide. The reaction typically involves the use of heat and a nickel or palladium catalyst such as but not limited to bis(triphenylphosphine)palladium(II) dichloride in a solvent such as but not limited to N-methylpyrrolidone, tetrahydrofuran, or mixtures thereof.

### Compositions

In another aspect, the present invention provides pharmaceutical compositions for modulating kinase activity in a humans and animals that will typically contain a compound of formula (I) and a pharmaceutically acceptable carrier.

Compounds having formula (I) may be administered, for example, bucally, ophthalmically, orally, osmotically, parenterally (intramuscularly, intraperintoneally intrasternally, intravenously, subcutaneously), rectally, topically, transdermally, vaginally and intraarterially as well as by intraarticular injection, infusion, and placement in the body, such as, for example, the vasculature.

Compounds having formula (I) may be administered with or without an excipient. Excipients include, but are not limited to, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising a compound having formula (I) to be administered orally include, but are not limited to, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, mixtures thereof and the like. Excipients for preparation of compositions comprising a compound having formula (I) to be administered ophthalmically or orally include, but are not limited to, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, mixtures thereof and the like. Excipients for preparation of compositions comprising a compound having formula (I) to be administered osmotically include, but are not limited to, chlorofluorohydrocarbons, ethanol, water, mixtures thereof and the like. Excipients for preparation of compositions comprising a compound having formula (I) to be administered parenterally include, but are not limited to, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, mixtures thereof and the like. Excipients for preparation of compositions comprising a compound having formula (I) to be administered rectally or vaginally include, but are not limited to, cocoa butter, polyethylene glycol, wax, mixtures thereof and the like.

The pharmaceutical composition and the method of the present invention may further comprise other therapeutically active compounds as noted herein which are usually applied in the treatment of the above-mentioned pathological conditions.

### Methods of Use

In another aspect, the present invention provides methods of using a compound or composition of the invention to treat or prevent a disease or condition involving mediation, overexpression or disregulation of kinases in a mammal. In particular, compounds of this invention are expected to have utility in treatment of diseases or conditions during which protein kinases such as any or all CDC-7 family members are expressed.

In one group of embodiments, diseases and conditions of humans or other animals that can be treated with inhibitors of kinases, include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenström's macroglobulinemia, testicular tumors, uterine cancer and Wilms' tumor.

The methods of the present invention typically involve administering to a subject in need of therapeutic treatment an effective amount of a compound of formula (I). Therapeutically effective amounts of a compound having formula (I) depend on recipient of treatment, disease treated and severity thereof, composition comprising it, time of administration, route of administration, duration of treatment, potency, rate of clearance and whether or not another drug is co-administered. The amount of a compound having formula (I) used to make a composition to be administered daily to a patient in a single dose or in divided doses is from about 0.03 to about 200 mg/kg body weight. Single dose compositions contain these amounts or a combination of submultiples thereof.

### Combination Therapy

The present invention further provides methods of using a compound or composition of the invention in combination with one or more additional active agents.

Compounds having Formula (I) are expected to be useful when used with alkylating agents, angiogenesis inhibitors, antibodies, antimetabolites, antimitotics, antiproliferatives, antivirals, aurora kinase inhibitors, apoptosis promoters (for example, Bcl-xL, Bcl-w and Bfl-1) inhibitors, activators of death receptor pathway, Bcr-Abl kinase inhibitors, BiTE (Bi-Specific T cell Engager) antibodies, antibody drug conjugates, biologic response modifiers, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNA's, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteosome inhibitors, purine analogs, pyrimidine analogs, receptor tyrosine kinase inhibitors, etinoids/deltoids plant alkaloids, small inhibitory ribonucleic acids (siRNAs), topoisomerase inhibitors, ubiquitin ligase inhibitors, and the like, and in combination with one or more of these agents.

BiTE antibodies are bi-specific antibodies that direct T-cells to attack cancer cells by simultaneously binding the two cells. The T-cell then attacks the target cancer cell. Examples of BiTE antibodies include adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bcl-2 has been shown to attenuate the induction of apoptosis by both perforin and granzyme B. These data suggest that inhibition of Bcl-2 could enhance the cytotoxic effects elicited by T-cells when targeted to cancer cells (V. R. Sutton, D. L. Vaux and J. A. Trapani, J. of Immunology 1997, 158 (12), 5783).

SiRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy chain DVD polypeptides and two light chain DVD polypeptides are referred to as DVD Ig's. Each half of a DVD Ig comprises a heavy chain DVD polypeptide, a light chain DVD polypeptide, and two antigen binding sites. Each binding site comprises a heavy chain variable domain and a light chain variable domain with a total of 6 CDRs involved in antigen binding per antigen binding site. Multispecific DVDs include DVD binding proteins that bind DLL4 and VEGF, or C-met and EFGR or ErbB3 and EGFR.

Alkylating agents include altretamine, AMD-473, AP-5280, apaziquone, bendamustine, brostallicin, busulfan, carboquone, carmustine (BCNU), chlorambucil, CLORETAZINE® (laromustine, VNP 40101M), cyclophosphamide, decarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, TREANDA® (bendamustine), treosulfan, rofosfamide and the like.

Angiogenesis inhibitors include endothelial-specific receptor tyrosine kinase (Tie-2) inhibitors, epidermal growth factor receptor (EGFR) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, vascular endothelial growth factor receptor tyrosine kinase (VEGFR) inhibitors and the like.

Antimetabolites include ALIMTA® (pemetrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflornithine, EICAR (5-ethynyl-1-β-D-ribofuranosylimidazole-4-carboxamide), enocitabine, ethnylcytidine, fludarabine, 5-fluorouracil alone or in combination with leucovorin, GEMZAR® (gemcitabine), hydroxyurea, ALKERAN®(melphalan), mercaptopurine, 6-mercaptopurine riboside, methotrexate, mycophenolic acid, nelarabine, nolatrexed, ocfosfate, pelitrexol, pentostatin, raltitrexed, Ribavirin, triapine, trimetrexate, S-1, tiazofurin, tegafur, TS-1, vidarabine, UFT and the like.

Antivirals include ritonavir, hydroxychloroquine and the like.

Aurora kinase inhibitors include ABT-348, AZD-1152, MLN-8054, VX-680, Aurora A-specific kinase inhibitors, Aurora B-specific kinase inhibitors and pan-Aurora kinase inhibitors and the like.

Bcl-2 protein inhibitors include AT-101 ((−)gossypol), GENASENSE® (G3139 or oblimersen (Bcl-2-targeting antisense oligonucleotide)), IPI-194, IPI-565, N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide) (ABT-737), N-(4-(4-((2-(4-chlorophenyl)-5,5-dimethyl-1-cyclohex-1-en-1-yl)methy 1)piperazin-1-yl)benzoyl)-4-(((1R)-3-(morpholin-4-yl)-1-((phenylsulfanyl)methyl)propyl)amino)-3-((trifluoromethyl)sulfonyl)benzene sulfonamide (ABT-263), GX-070 (obatoclax) and the like.

Bcr-Abl kinase inhibitors include DASATINIB® (BMS-354825), GLEEVEC® (imatinib) and the like.

CDK inhibitors include AZD-5438, BMI-1040, BMS-032, BMS-387, CVT-2584, flavopyridol, GPC-286199, MCS-5A, PD0332991, PHA-690509, seliciclib (CYC-202, R-roscovitine), ZK-304709 and the like.

COX-2 inhibitors include ABT-963, ARCOXIA® (etoricoxib), BEXTRA® (valdecoxib), BMS347070, CELEBREX® (celecoxib), COX-189 (lumiracoxib), CT-3, DERAMAXX® (deracoxib), JTE-522, 4-methyl-2-(3,4-dimethylphenyl)-1-(4-sulfamoylphenyl-1H-pyrrole), MK-663 (etoricoxib), NS-398, parecoxib, RS-57067, SC-58125, SD-8381, SVT-2016, S-2474, T-614, VIOXX® (rofecoxib) and the like.

EGFR inhibitors include ABX-EGF, anti-EGFR immunoliposomes, EGF-vaccine. EMD-7200, ERBITUX® (cetuximab), HR3, IgA antibodies, IRESSA®(gefitinib), TARCEVA® (erlotinib or OSI-774), TP-38, EGFR fusion protein, TYKERB® (lapatinib) and the like.

ErbB2 receptor inhibitors include CP-724-714, CI-1033 (canertinib), HERCEPTIN® (trastuzumab), TYKERB® (lapatinib), OMNITARG® (2C4, petuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her2IgG3, AS HER2 trifunctional bispecific antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17-AAG-nab, 17-AAG, CNF-101, CNF-1010, CNF-2024, 17-DMAG, geldanamycin, IPI-504, KOS-953, MYCOGRAB® (human recombinant antibody to HSP-90), NCS-683664, PU24FCI, PU-3, radicicol, SNX-2112, STA-9090 VER49009 and the like.

Inhibitors of inhibitors of apoptosis proteins include HGS1029, GDC-0145, GDC-0152, LCL-161, LBW-242 and the like.

Antibody drug conjugates include anti-CD22-MC-MMAF, anti-CD22-MC-MMAE, anti-CD22-MCC-DM1, CR-011-veMMAE, PSMA-ADC, MEDI-547, SGN-19Am SGN-35, SGN-75 and the like.

Activators of death receptor pathway include TRAIL, antibodies or other agents that target TRAIL or death receptors (e.g., DR4 and DR5) Such as Apomab, conatumumab, ETR2-ST01, GDC0145, (lexatumumab), HGS-1029, LBY-135, PRO-1762 and trastuzumab.

Kinesin inhibitors include Eg5 inhibitors such as AZD4877, ARRY-520; CENPE inhibitors such as GSK923295A and the like.

JAK-2 inhibitors include CEP-701 (lesaurtinib), XL019 and INCB018424 and the like.

MEK inhibitors include ARRY-142886, ARRY-438162 PD-325901, PD-98059 and the like.

mTOR inhibitors include AP-23573, CCI-779, everolimus, RAD-001, rapamycin, temsirolimus, ATP-competitive TORC1/TORC2 inhibitors, including PI-103, PP242, PP30, Torin 1 and the like.

Non-steroidal anti-inflammatory drugs include AMIGESIC® (salsalate), DOLOBID® (diflunisal), MOTRIN® (ibuprofen), ORUDIS® (ketoprofen), RELAFEN® (nabumetone), FELDENE® (piroxicam), ibuprofen cream, ALEVE® (naproxen) and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), DAYPRO® (oxaprozin) and the like.

PDGFR inhibitors include C-451, CP-673, CP-868596 and the like.

Platinum chemotherapeutics include cisplatin, ELOXATIN® (oxaliplatin) eptaplatin, lobaplatin, nedaplatin, PARAPLATIN® (carboplatin), satraplatin, picoplatin and the like.

Polo-like kinase inhibitors include BI-2536 and the like.

Phosphoinositide-3 kinase (PI3K) inhibitors include wortmannin, LY294002, XL-147, CAL-120, ONC-21, AEZS-127, ETP-45658, PX-866, GDC-0941, BGT226, BEZ235, XL765 and the like.

Thrombospondin analogs include ABT-510, ABT-567, ABT-898, TSP-1 and the like.

VEGFR inhibitors include AVASTIN® (bevacizumab), ABT-869, AEE-788, ANGIOZYME™ (a ribozyme that inhibits angiogenesis (Ribozyme Pharmaceuticals (Boulder, Colo.) and Chiron, (Emeryville, Calif.)), axitinib (AG-13736), AZD-2171, CP-547,632, IM-862, MACUGEN (pegaptamib), NEXAVAR® (sorafenib, BAY43-9006), pazopanib (GW-786034), vatalanib (PTK-787, ZK-222584), SUTENT® (sunitinib, SU-11248), VEGF trap, ZACTIMA™ (vandetanib, ZD-6474), GA101, ofatumumab, ABT-806 (mAb-806), ErbB3 specific antibodies, BSG2 specific antibodies, DLL4 specific antibodies and C-met specific antibodies, and the like.

Antibiotics include intercalating antibiotics aclarubicin, actinomycin D, amrubicin, annamycin, adriamycin, BLENOXANE® (bleomycin), daunorubicin, CAELYX® or MYOCET® (liposomal doxorubicin), elsamitrucin, epirbucin, glarbuicin, ZAVEDOS® (idarubicin), mitomycin C, nemorubicin, neocarzinostatin, peplomycin, pirarubicin, rebeccamycin, stimalamer, streptozocin, VALSTAR® (valrubicin), zinostatin and the like.

Topoisomerase inhibitors include aclarubicin, 9-aminocamptothecin, amonafide, amsacrine, becatecarin, belotecan, BN-80915, CAMPTOSAR® (irinotecan hydrochloride), camptothecin, CARDIOXANE® (dexrazoxine), diflomotecan, edotecarin, ELLENCE® or PHARMORUBICIN® (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include AVASTIN® (bevacizumab), CD40-specific antibodies, chTNT-1/B, denosumab, ERBITUX® (cetuximab), HUMAX-CD4® (zanolimumab), IGFIR-specific antibodies, lintuzumab, PANOREX® (edrecolomab), RENCAREX® (WX G250), RITUXAN® (rituximab), ticilimumab, trastuzimab, CD20 antibodies types I and II and the like.

Hormonal therapies include ARIMIDEX® (anastrozole), AROMASIN® (exemestane), arzoxifene, CASODEX® (bicalutamide), CETROTIDE® (cetrorelix), degarelix, deslorelin, DESOPAN® (trilostane), dexamethasone, DROGENIL® (flutamide), EVISTA® (raloxifene), AFEMA™ (fadrozole), FARESTON® (toremifene), FASLODEX® (fulvestrant), FEMARA® (letrozole), formestane, glucocorticoids, HECTOROL® (doxercalciferol), RENAGEL® (sevelamer carbonate), lasofoxifene, leuprolide acetate, MEGACE® (megesterol), MIFEPREX® (mifepristone), NILANDRON™ (nilutamide), NOLVADEX® (tamoxifen citrate), PLENAXIS™ (abarelix), prednisone, PROPECIA® (finasteride), rilostane, SUPREFACT® (buserelin), TRELSTAR® (luteinizing hormone releasing hormone (LHRH)), VANTAS® (Histrelin implant), VETORYL® (trilostane or modrastane), ZOLADEX® (fosrelin, goserelin) and the like.

Deltoids and retinoids include seocalcitol (EB1089, CB1093), lexacalcitrol (KH1060), fenretinide, PANRETIN® (aliretinoin), ATRAGEN® (liposomal tretinoin), TARGRETIN® (bexarotene), LGD-1550 and the like.

PARP inhibitors include ABT-888 (veliparib), olaparib, KU-59436, AZD-2281, AG-014699, BSI-201, BGP-15, INO-1001, ONO-2231 and the like.

Plant alkaloids include, but are not limited to, vincristine, vinblastine, vindesine, vinorelbine and the like.

Proteasome inhibitors include VELCADE® (bortezomib), MG132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, ACTIMMUNE® (interferon gamma-1b) or interferon gamma-n1, combinations thereof and the like. Other agents include ALFAFERONE®, (IFN-α), BAM-002 (oxidized glutathione), BEROMUN® (tasonermin), BEXXAR® (tositumomab), CAMPATH® (alemtuzumab), CTLA4 (cytotoxic lymphocyte antigen 4), decarbazine, denileukin, epratuzumab, GRANOCYTE® (lenograstim), lentinan, leukocyte alpha interferon, imiquimod, MDX-010 (anti-CTLA-4), melanoma vaccine, mitumomab, molgramostim, MYLOTARG™ (gemtuzumab ozogamicin), NEUPOGEN® (filgrastim), OncoVAC-CL, OVAREX® (oregovomab), pemtumomab (Y-muHMFGI), PROVENGE® (sipuleucel-T), sargaramostim, sizofilan, teceleukin, THERACYS® (Bacillus Calmette-Guerin), ubenimex, VIRULIZIN® (immunotherapeutic, Lorus Pharmaceuticals), Z-100 (Specific Substance of Maruyama (SSM)), WF-10 (Tetrachlorodecaoxide (TCDO)), PROLEUKIN® (aldesleukin), ZADAXIN® (thymalfasin), ZENAPAX® (daclizumab), ZEVALIN® (90Y-Ibritumomab tiuxetan) and the like.

Biological response modifiers are agents that modify defense mechanisms of living organisms or biological responses, such as survival, growth or differentiation of tissue cells to direct them to have anti-tumor activity and include krestin, lentinan, sizofiran, picibanil PF-3512676 (CpG-8954), ubenimex and the like.

Pyrimidine analogs include cytarabine (ara C or Arabinoside C), cytosine arabinoside, doxifluridine, FLUDARA® (fludarabine), 5-FU (5-fluorouracil), floxuridine, GEMZAR® (gemcitabine), TOMUDEX® (ratitrexed), TROXATYL™ (triacetyluridine troxacitabine) and the like.

Purine analogs include LANVIS® (thioguanine) and PURI-NETHOL® (mercaptopurine).

Antimitotic agents include batabulin, epothilone D (KOS-862), N-(2-((4-hydroxyphenyl)amino)pyridin-3-yl)-4-methoxybenzenesulfonamide, ixabepilone (BMS 247550), paclitaxel, TAXOTERE® (docetaxel), PNU100940 (109881), patupilone. XRP-9881 (larotaxel), vinflunine, ZK-EPO (synthetic epothilone) and the like.

Ubiquitin ligase inhibitors include MDM2 inhibitors, such as nutlins, NEDD8 inhibitors such as MLN4924 and the like.

Compounds of this invention can also be used as radiosensitizers that enhance the efficacy of radiotherapy. Examples of radiotherapy include external beam radiotherapy, teletherapy, brachytherapy and sealed, unsealed source radiotherapy and the like.

Additionally, compounds having Formula (I) may be combined with other chemotherapeutic agents such as ABRAXANE™ (ABI-007), ABT-100 (farnesyl transferase inhibitor), ADVEXIN® (Ad5CMV-p53 vaccine), ALTOCOR® or MEVACOR® (lovastatin), AMPLIGEN® (poly I:poly C12U, a synthetic RNA), APTOSYN® (exisulind), AREDIA® (pamidronic acid), arglabin, L-asparaginase, atamestane (1-methyl-3,17-dione-androsta-1,4-′ diene), AVAGE® (tazarotene), AVE-8062 (combreastatin derivative) BEC2 (mitumomab), cachectin or cachexin (tumor necrosis factor), canvaxin (vaccine), CEAVAC® (cancer vaccine), CELEUK® (celmoleukin), CEPLENE® (histamine dihydrochloride), CERVARIX® (human papillomavirus vaccine), CHOP® (C: CYTOXAN® (cyclophosphamide); H: ADRIAMYCIN® (hydroxydoxorubicin); 0: Vincristine (ONCOVIN®); P: prednisone), CYPAT™ (cyproterone acetate), combrestatin A4P, DAB(389)EGF (catalytic and translocation domains of diphtheria toxin fused via a His-Ala linker to human epidermal growth factor) or TransMID-107R™ (diphtheria toxins), dacarbazine, dactinomycin, 5,6-dimethylxanthenone-4-acetic acid (DMXAA), eniluracil, EVIZON™ (squalamine lactate), DIMERICINE® (T4N5 liposome lotion), discodermolide, DX-8951f (exatecan mesylate), enzastaurin, EP0906 (epithilone B), GARDASIL® (quadrivalent human papillomavirus (Types 6, 11, 16, 18) recombinant vaccine), GASTRIMMUNE®, GENASENSE®, GMK (ganglioside conjugate vaccine), GVAX® (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, JUNOVAN™ or MEPACT™ (mifamurtide), lonafarnib, 5,10-methylenetetrahydrofolate, miltefosine (hexadecylphosphocholine), NEOVASTAT®(AE-941), NEUTREXIN® (trimetrexate glucuronate), NIPENT® (pentostatin), ONCONASE® (a ribonuclease enzyme), ONCOPHAGE® (melanoma vaccine treatment), ONCOVAX® (IL-2 Vaccine), ORATHECIN™ (rubitecan), OSIDEM® (antibody-based cell drug), OVAREX® MAb (murine monoclonal antibody), paclitaxel, PANDIMEX™ (aglycone saponins from ginseng comprising 20(S)protopanaxadiol (aPPD) and 20(S)protopanaxatriol (aPPT)), panitumumab, PANVAC®-VF (investigational cancer vaccine), pegaspargase, PEG Interferon A, phenoxodiol, procarbazine, rebimastat, REMOVAB® (catumaxomab), REVLIMID® (lenalidomide), RSR13 (efaproxiral), SOMATULINE® LA (lanreotide), SORIATANE® (acitretin), staurosporine (Streptomyces staurospores), talabostat (PT100), TARGRETIN® (bexarotene), TAXOPREXIN® (DHA-paclitaxel), TELCYTA® (canfosfamide, TLK286), temilifene, TEMODAR® (temozolomide), tesmilifene, thalidomide, THERATOPE® (STn-KLH), thymitaq (2-amino-3,4-dihydro-6-methyl-4-oxo-5-(4-pyridylthio)quinazoline dihydrochloride), TNFERADE™ (adenovector: DNA carrier containing the gene for tumor necrosis factor-α), TRACLEER® or ZAVESCA® (bosentan), tretinoin (Retin-A), tetrandrine, TRISENOX® (arsenic trioxide), VIRULIZIN®, ukrain (derivative of alkaloids from the greater celandine plant), vitaxin (anti-alphavbeta3 antibody), XCYTRIN® (motexafin gadolinium), XINLAY™ (atrasentan), XYOTAX™ (paclitaxel poliglumex), YONDELIS® (trabectedin), ZD-6126, ZINECARD® (dexrazoxane), ZOMETA® (zolendronic acid), zorubicin and the like.

### EXAMPLES

### Example 1

### 2-(2,6-dichlorobenzyl)-4-{[2-methoxy-4-(4-methylpiperazin-1-yl)phenyl]amino}-1,6-naphthyridin-5 (6H)-one

### Example 1a

### ethyl 4-(2,6-dichlorophenyl)-3-oxobutanoate

To a solution of 2-(2,6-dichlorophenyl)acetic acid (10 g, 48.8 mmol) in dry tetrahydrofuran (100 mL) was added N,N-carbonyldiimidazole (9.5 g, 58.5 mmol). The mixture was stirred at ambient temperature for 1 hour and magnesium chloride (4.6 g, 48.8 mmol) and ethyl potassium malonate (12.4 g, 73.2 mmol) were added. The mixture was refluxed at 70° C. for 18 hours. The mixture was diluted with water (500 mL) and the solution was acidified with concentrated hydrochloric acid to pH=1. The mixture was extracted with ethyl acetate (2×200 mL) and the combined organic layers were dried over anhydrous sodium sulfate, filtered and concentrated to afford the crude product which was used directly in the next step without further purification. MS: 275 (M+H⁺).

### Example 1B

### methyl 3-amino-4-(2,6-dichlorophenyl)butanoate

To a solution of the product of EXAMPLE 1A (15 g, crude) in methanol (200 mL) was added ammonium acetate (42 g, 545.4 mmol), magnesium sulfate (20 g) and sodium cyanoborohydride (6.8 g, 109.1 mmol). The mixture was refluxed at 70° C. for 18 hours. The mixture was poured into water (600 mL) and the solution extracted with ethyl acetate (3×200 mL). The combined organic layers were concentrated and the residue was diluted with ethyl acetate (300 mL) and the solution extracted with 1 N hydrochloric acid (300 mL). The aqueous phase was separated and 50% aqueous sodium hydroxide was added to adjust the pH to 8-9. The basic solution was extracted with ethyl acetate (3×150 mL) and the combined organic layers were dried over anhydrous sodium sulfate, filtered and concentrated to afford the title compound. ¹H NMR (CDCl₃) δ ppm 7.3-7.2 (m, 2H), 7.09 (d, J=7.8 Hz, 1H), 3.7-3.6 (m, 4H), 3.06 (d, J=6.9 Hz, 2H), 2.6-2.4 (m, 2H), 1.84 (brs, 2H).

### Example 1C

### ethyl 3-(1-(methoxycarbonyl)-3-(2,6-dichlorophenyl)propan-2-ylamino)but-2-enoate

To a solution of the product of EXAMPLE 1B (5 g, 19.2 mmol) in dry toluene (50 mL) was added acetic acid (2.23 g, 38.3 mmol). To the resulting suspension was added ethyl acetoacetate (2.99 g, 23.0 mmol) and magnesium sulfate (5 g) and the mixture refluxed at 110° C. for 3 hours. After cooling to ambient temperature, the mixture was partitioned between ethyl acetate (100 mL) and saturated aqueous sodium bicarbonate (200 mL). The organic layer was dried over anhydrous sodium sulfate, filtered and concentrated to afford the crude product which was used directly in the next step without further purification.

### Example 1D

### ethyl 6-(2,6-dichlorobenzyl)-1,4,5,6-tetrahydro-2-methyl-4-oxopyridine-3-carboxylate

To a solution of the crude product of EXAMPLE 1C (7.4 g) in dry tetrahydrofuran (100 mL) was added potassium tert-butoxide (4.45 g, 39.7 mmol) and the mixture stirred at ambient temperature for 18 hours. The mixture was concentrated and the residue partitioned between ethyl acetate (200 mL) and ice-water (300 mL). The aqueous phase was separated and extracted with ethyl acetate (2×100 mL). The combined organic layers were washed with brine (100 mL), dried over anhydrous sodium sulfate, filtered and concentrated to afford the title compound. ¹H NMR (CDCl₃) δ ppm 7.3-7.2 (m, 2H), 7.15 (dd, J=8.6, 7.4 Hz, 1H), 5.94 (brs, 1H), 4.22 (q, J=7.2 Hz, 2H), 4.1-4.0 (m, 1H), 3.41 (dd, J=13.5, 6.0 Hz, 1H), 3.13=13.5, 6.0 Hz, 1H), 2.6-2.4 (m, 2H), 2.27 (s, 3H), 1.31 (t, J=7.5 Hz, 3H). MS: 342 (M+H⁺).

### Example 1E

### ethyl 6-(2,6-dichlorobenzyl)-1,4-dihydro-2-methyl-4-oxopyridine-3-carboxylate

To a solution of the product of EXAMPLE 1D (4.5 g, 13.2 mmol) in dry tetrahydrofuran (50 mL) was added dropwise a solution of 2,3-dichloro-5,6-dicyano-1,4-benzoquinone (3.6 g, 15.8 mmol) in dry tetrahydrofuran (50 mL) over a period of 5 minutes at ambient temperature and the mixture was stirred at ambient temperature for 18 hours. The majority of the solvent was removed and the residue was partitioned between saturated aqueous sodium bicarbonate (300 mL) and dichloromethane (200 mL). The aqueous phase was extracted with dichloromethane (2×200 mL) and the combined organic layers dried over anhydrous sodium sulfate, filtered and concentrated to afford the crude product which was used directly in the next step without further purification. ¹H NMR (DMSO-d₆) δ ppm 11.8 (brs., 1H), 7.7-7.6 (m, 2H), 7.48 (dd, J=8.6, 7.7 Hz, 1H), 5.31 (brs., 1H), 4.3-4.1 (m, 4H), 2.32 (s, 3H), 1.30 (t, J=7.1 Hz, 3H). MS: 340 (M+H⁺).

### Example 1F

### ethyl 6-(2,6-dichlorobenzyl)-4-chloro-2-methylpyridine-3-carboxylate

A solution of the product of EXAMPLE 1E (4.7 g, crude) in phosphorus oxychloride (20 mL) was stirred at 102° C. for 2.5 hours. After cooling, the mixture was added slowly to ice-water (300 mL). The solution was extracted with ethyl acetate (3×200 mL) and the combined organic layers dried over anhydrous sodium sulfate, filtered and concentrated. The residue was purified by chromatography eluting with 50/1 petroleum ether/ethyl acetate to afford the title compound. MS: 358 (M+H⁺).

### Example 1G

### ethyl 6-(2,6-dichlorobenzyl)-4-(2-methoxy-4-(4-methylpiperazin-1-yl)phenylamino)-2-methylnicotinate

A solution of the product of EXAMPLE 1F (500 mg, 1.40 mmol), 2-methoxy-4-(4-methylpiperazin-1-yl)aniline (321 mg, 1.68 mmol) and p-toluenesulfonic acid (20 mg, cat.) in n-butanol (10 mL) was heated at 100° C. for 18 hours. After cooling, the mixture was poured into saturated aqueous sodium bicarbonate (100 mL) and the solution was extracted with ethyl acetate (3×50 mL). The combined organic layers were dried over anhydrous sodium sulfate, filtered and concentrated. The residue was purified by flash chromatography on silica gel (200-300 mesh) eluting with 20/1 dichloromethane/methanol to afford the title compound. MS: 272.2 (M/2+H⁺).

### Example 1H

### 2-(2,6-dichlorobenzyl)-4-{[2-methoxy-4-(4-methylpiperazin-1-yl)phenyl]amino}-1,6-naphthyridin-5(6H)-one

To a suspension of sodium hydride (85 mg, 2.13 mmol, 60%) in dry N,N-dimethylformamide (2 mL) was added dropwise a solution of the product of EXAMPLE 1G (360 mg, 0.664 mmol) in dry N,N-dimethylformamide (3 mL) at ambient temperature under nitrogen. After stirring at ambient temperature for 15 minutes, 1,3,5-triazine (108 mg, 1.33 mmol) in dry N,N-dimethylformamide (1 mL) was added and the mixture heated at 100° C. for 18 hours. After cooling to ambient temperature, the mixture was poured into saturated aqueous ammonium chloride (60 mL) and the solution extracted with ethyl acetate (3×40 mL). The combined organic layers were dried over anhydrous sodium sulfate, filtered and concentrated. The residue was purified by preparative thin layer chromotography (8:1 dichloromethane/methanol with NH₃/methanol as additive), followed by preparative HPLC (acetonitrile/water, containing 1% trifluoroacetic acid) to afford the title compound as a mono-trifluoroacetate salt. ¹H NMR (CD₃OD) δ ppm 11.12 (brs, 1H), 7.30-7.27 (m, 2H), 7.16-7.11 (m, 1H), 7.02 (d, J=8.4 Hz, 1H), 6.84-6.82 (m, 1H), 6.39-6.33 (m, 2H), 6.00 (s. 1H), 4.49 (s, 2H), 3.77 (s, 3H), 3.28 (brs, 4H), 2.86 (brs, 4H), 2.55 (s, 3H). MS: 262.6 (M/2+H⁺).

### Example 2

### 2-(2,6-dichlorobenzyl)-4-{[2-methoxy-4-(piperazin-1-yl)phenyl]amino}-1,6-naphthyridin-5(6H)-one

### Example 2A

### tert-butyl 4-(4-(3-(ethoxycarbonyl)-6-(2,6-dichlorobenzyl)-2-methylpyridin-4-ylamino)-3-methoxyphenyl)piperazine-1-carboxylate

The title compound was obtained using the procedure described in EXAMPLE 1G substituting tert-butyl 4-(4-amino-3-methoxyphenyl)piperazine-1-carboxylate in place of 2-methoxy-4-(4-methylpiperazin-1-yl)aniline. MS: 629 (M+H⁺).

### Example 2B

### tert-butyl 4-(4-(2-(2,6-dichlorobenzyl)-5,6-dihydro-5-oxo-1,6-naphthyridin-4-ylamino)-3-methoxyphenyl)piperazine-1-carboxylate

The title compound was synthesized using the procedure described in EXAMPLE 1H, substituting EXAMPLE 2A for EXAMPLE 1G. MS: 610 (M+H⁺).

### Example 2C

### 2-(2,6-dichlorobenzyl)-4-{[2-methoxy-4-(piperazin-1-yl)phenyl]amino}-1,6-naphthyridin-5(6H)-one

To a solution of the product of EXAMPLE 2B (300 mg, 0.493 mmol) in dry dichloromethane (10 mL) was added trifluoroacetic acid (10 mL) slowly at ambient temperature and the mixture stirred for 18 hours. The solvent was removed and the residue partitioned between saturated aqueous sodium bicarbonate (30 mL) and ethyl acetate (20 mL). The organic phase was separated, dried over anhydrous sodium sulfate, filtered and concentrated to afford the crude compound, which was purified by preparative thin layer chromotography (3/1 dichloromethane/methanol), followed by preparative HPLC (acetonitrile/water, containing 0.1% trifluoroacetic acid) to give the title compound as a mono trifluoroacetate salt. ¹H NMR (CD₃OD) δ ppm 12.04 (brs, 1H), 7.76 (d, J=7.2 Hz, 1H), 7.5-7.4 (m, 2H), 7.37 (dd, J=9.0, 6.9 Hz, 1H), 7.09 (d, J=8.7 Hz, 1H), 6.7-6.6 (m, 2H), 6.59 (dd, J=8.7, 2.4 Hz, 1H), 5.81 (s, 1H), 4.88 (s, 2H), 3.73 (s, 3H), 3.6-3.3 (m, 8H). MS: 255.6 (M/2+H⁺).

### Example 3

### 2-(2,6-dichlorobenzyl)-4-{[4-(piperazin-1-yl)phenyl]amino}-1,6-naphthyridin-5(6H)-one

### Example 3A

### tert-butyl 4-(4-(3-(ethoxycarbonyl)-6-(2,6-dichlorobenzyl)-2-methylpyridin-4-ylamino)phenyl)piperazine-1-carboxylate

The title compound was obtained using the procedure described in EXAMPLE 1G using tert-butyl 4-(4-aminophenyl)piperazine-1-carboxylate in place of 2-methoxy-4-(4-methylpiperazin-1-yl)aniline. MS: 599 (M+H⁺).

### Example 3B

### tert-butyl 4-(4-(2-(2,6-dichlorobenzyl)-5,6-dihydro-5-oxo-1,6-naphthyridin-4-ylamino)phenyl)piperazine-1-carboxy late

The title compound was obtained using the procedure described in EXAMPLE 1H. MS: 580.2 (M+H⁺).

### Example 3C

### 2-(2,6-dichlorobenzyl)-4-([4-(piperazin-1-yl)phenyl]aminol-1,6-naphthyridin-5(6H)-one

The title compound was obtained using the procedure described in EXAMPLE 2C, substituting EXAMPLE 3B for EXAMPLE 2B. ¹H NMR (CD₃OD) δ ppm 7.74 (dd, J=7.2, 0.9 Hz, 1H), 7.5-7.4 (m, 2H), 7.4-7.3 (m, 1H), 7.12 (d, J=8.7 Hz, 2H), 7.03 (d, J=8.7 Hz, 2H), 6.63 (dd, J=7.2, 0.9 Hz, 1H), 6.03 (d, J=0.6 Hz, 1H), 4.47 (s, 2H), 3.5-3.3 (m, 8H). MS: 240.6 (M/2+H).

### Example 4

### 6-(2,6-dichlorobenzyl)-8-{[2-methoxy-4-(4-methylpiperazin-1-yl)phenyl]amino}-2,7-naphthyridin-1(2H)-one

### Example 4A

### 2,6-dihydroxy-4-methylnicotinonitrile

Potassium hydroxide (14 g, 261 mmol) was added to a solution of 2-cyanoacetamide (20 g, 238 mmol) and ethyl 3-oxobutanoate (30.8 g, 238 mmol) in methanol (600 mL) and the mixture was refluxed for 12 hours. The mixture was poured into water (500 mL) and acidified to pH˜1 using concentrated hydrochloric acid. The suspension was stirred at ambient temperature for 12 hours and the precipitate was filtered, washed with water (500 mL) and dried under vacuum to afford the title compound. ¹H NMR (DMSO-d₆) δ ppm 5.57 (s, 1H), 2.22 (s, 3H).

### Example 4B

### 2,6-dichloro-4-methylnicotinonitrile

The product of EXAMPLE 4A (5 g, 33.3 mmol) and phosphorus oxychloride (9.3 mL, 0.1 mol) were heated in a sealed tube at 160° C. for 7 hours. The mixture was cooled, poured into ice-water and stirred at ambient temperature for 1 hours. The precipitate was filtered, washed with water and dried in vacuo to provide the title compound. ¹H-NMR: (DMSO-d₆) δ ppm 7.81 (s, 1H), 2.53 (s, 3H).

### Example 4C

### (E)-2,6-dichloro-4-(2-(dimethylamino)vinyl)nicotinonitrile

N,N-dimethylformamide dimethyl acetal (0.48 g, 4 mmol) was added dropwise to a solution of EXAMPLE 4B (0.5 g, 2.69 mmol) in N,N-dimethylformamide (10 mL) and the mixture stirred at 100° C. for 2 hours. After cooling to room temperature, the mixture was concentrated in vacuo and the residue purified by flash chromatography on silica gel (200-300 mesh) eluting with dichloromethane to afford the title compound. ¹H-NMR (CDCl₃) δ 7.35 (d, J=13.2 Hz, 1H), 7.05 (s, 1H), 5.26 (d, J=13.2 Hz, 1H), 3.06 (s, 6H).

### Example 4D

### 6,8-dichloro-2,7-naphthyridin-1(2H)-one

Concentrated hydrochloric acid (15 mL) and EXAMPLE 4C (3 g, 12.5 mmol) were heated in a sealed tube at 45° C. overnight. Ice (10 g) was added and the precipitate was filtered, washed with cold water and dried in vacuo to give the title compound. ¹H NMR (DMSO-d₆) δ ppm 11.76 (s, 1H), 7.76 (s, 1H), 7.52 (d, J=6.9 Hz, 1H), 6.53 (d, J=6.9 Hz, 1H).

### Example 4E

### 6-chloro-8-(2-methoxy-4-(4-methylpiperazin-1-yl)phenylamino)-2,7-naphthyridin-1(2H)-one

EXAMPLE 4D (0.2 g, 0.93 mmol) and 2-methoxy-4-(4-methylpiperazin-1-yl)aniline (0.21 g, 0.93 mmol) in anhydrous N-methylpyrrolidone (0.5 mL) were heated in a microwave (CEM Discover-S, Model Number: 908860) at 140° C. for 1 hour. The mixture was concentrated in vacuo and the residue purified by flash chromatography on silica gel (200-300 mesh) eluting with 1/40 methanol/dichloromethane to afford the title compound.

### Example 4F

### 6-(2,6-dichlorobenzyl)-8-{[2-methoxy-4-(4-methylpiperazin-1-yl)phenyl]amino}-2,7-naphthyridin-1 (2H)-one

A solution of the product of EXAMPLE 4E (0.34 g, 0.85 mmol) in N-methylpyrrolidone (1 mL) and tetrahydrofuran (7 mL) was degassed with nitrogen. Catalytic bis(triphenylphosphine)palladium(II) dichloride and (2,6-dichlorobenzyl)zinc(II) bromide (1.0 M in tetrahydrofuran, 3.4 mL, 3.4 mmol) were added and the mixture stirred at 60-65° C. for 24 hours. The mixture was filtered and washed with methanol. The filtrate was concentrated in vacuo and the residue partitioned between ethyl acetate and water. The organic phase was washed with brine, dried over anhydrous sodium sulfate, filtered and concentrated. The residue was purified by preparative HPLC (acetonitrile/water containing 0.1% trifluoroacetic acid) to afford the title compound as a mono trifluoroacetate salt. ¹H NMR (DMSO-d₆) δ ppm 11.81 (s, 1H), 11.42 (d, J=6.3 Hz, 1H), 8.20 (s, 1H), 8.02 (d, 0.1=8.7 Hz, 1H), 7.58 (s, 1H), 7.57 (s, 1H), 7.42-7.37 (m, 1H), 7.33-7.29 (m, 1H), 6.59 (s, 2H), 6.38 (d, 0.1=7.2 Hz, 1H), 6.18 (dd, J=2.4, 9 Hz, 1H), 4.36 (s, 2H), 3.83 (s, 3H), 3.16-3.02 (m, 4H), 2.50-2.42 (m, 4H), 2.24 (s, 3H). MS: 524 (M+1).

### Example 5

### 7-(2,6-dichlorobenzyl)-5-{[2-methoxy-4-(4-methylpiperazin-1-yl)phenyl]amino}pyrido[4,3-d]pyrimidin-4(3H)-one

### Example 5A

### 2,6-dichloropyridine-4-carboxylic acid

A mixture of 2,6-dihydroxypyridine-4-carboxylic acid (15.1 g, 100 mmol) and phosphoryl trichloride (45 ml) was heated for 6 hours at 160-165° C. in a 200 mL sealed tube. After cooling to ambient temperature, the mixture was poured into crushed ice (300 g) and stirred for 1 hours. The mixture was extracted with ethyl acetate (5×60 mL) and the combined organic layers were dried over anhydrous sodium sulfate, filtered and concentrated in vacuo to give the of crude product which was recrystallized from 40 mL of 2/1 ethyl acetate/petroleum ether to afford the title compound. ¹H NMR (DMSO-d₆) δ ppm 7.89 (s, 2H). MS: 192 (M+1).

### Example 5B

### tert-butyl 2,6-dichloropyridin-4-ylcarbamate

To a solution of the product of EXAMPLE 5A (18.0 g, 93.7 mmol) in anhydrous tert-butanol (200 mL) was added diphenylphosphoryl azide (27.1 g, 98 mmol) and N,N-diisopropylethylamine (24.2 g, 187.5 mmol) and the mixture was refluxed under nitrogen for 6 hours. The mixture was concentrated in vacuo and the residue was dissolved in ethyl acetate, washed with ammonium chloride solution and dried over sodium sulfate. Filtration, concentration of the filtrate, and purification by flash chromatography on silica gel using 10/1 petroleum ether/ethyl acetate afforded the title compound. ¹H NMR (DMSO-d₆) δ ppm 10.33 (s, 1H), 7.49 (s, 2H), 1.48 (s, 9H).

### Example 5C

### 4-(tert-butoxycarbonylamino)-2,6-dichloronicotinic acid

N,N,N′,N′-tetramethylethylenediamine (1.7 g, 14.7 mmol) was added to a solution of EXAMPLE 5B (1.84 g, 7.0 mmol) in anhydrous tetrahydrofuran (35 mL). The mixture was degassed and recharged with nitrogen 4 times and cooled to −60° C. n-Butyl lithium (6.4 mL, 16.1 mmol) was added dropwise and the mixture stirred at −60° C. for 2 hours. Dry carbon dioxide gas was bubbled into this solution and the mixture stirred overnight. The mixture was quenched with water and the solvent removed in vacuo. The residue was diluted with water and washed with 2/1 petroleum ether/ethyl acetate (2×20 mL). The aqueous phase was acidified to pH=2 with concentrated hydrochloric acid and the mixture extracted with ethyl acetate. The combined organic layers were dried with sodium sulfate, filtered and concentrated in vacuo to give the title compound. ¹H NMR (DMSO-d₆) δ ppm 9.83 (s, 1H), 7.93 (s, 1H), 1.47 (s, 9H).

### Example 5D

### 4-amino-2,6-dichloropyridine-3-carboxamide

To a solution of the product of EXAMPLE 5C (11.86 g, 38.6 mmol) of N,N-dimethylformamide (120 mL) was added 1,1′-carbonyldiimidazole (6.89 g, 42.5 mmol) and the mixture was stirred at 60° C. for 2 hours and then was cooled to 0-5° C. Ammonia gas was bubbled into the solution and the mixture was stirred overnight. The mixture was poured into 800 mL water and was extracted with ethyl acetate. The organic layers were dried over anhydrous sodium sulfate, filtered and concentrated in vacuo. The residue was purified by flash chromatography on silica gel (200-300 mesh) using 50/1 dichloromethane/methanol to afford the title compound. ¹H NMR (DMSO-d₆) δ ppm 7.97 (brs., 1H), 7.71 (brs., 1H), 6.61 (s, 1H), 6.59 (brs., 2H).

### Example 5E

### 5,7-dichloropyrido[4,3-d]pyrimidin-4(3H)-one

A solution of the product of EXAMPLE 5D (2.65 g, 13 mmol) in 15 mL of triethyl orthoformate was refluxed under nitrogen for 6 hours. After cooling to ambient temperature, the solid was filtered and washed with 1/1 petroleum ether/ethyl acetate (5 mL) to give the title compound. ¹H NMR (DMSO-d₆) 6 ppm 12.84 (br s, 1H), 8.33 (s, 1H), 7.72 (s, 1H).

### Example 5F

### 5-(2-methoxy-4-(4-methylpiperazin-1-yl)phenylamino)-7-chloropyrido[4,3-d]pyrimidin-4(3H)-one

A solution of the product of EXAMPLE 5E (300 mg, 1.4 mmol), 2-methoxy-4-(4-methylpiperazin-1-yl)aniline (338 mg, 1.53 mmol) and triethylamine (421 mg, 4.17 mmol) in 1,4-dioxane (30 mL) was stirred at 105° C. under nitrogen for 12 hours. The solvent was removed under vacuum and the residue was washed with sodium bicarbonate solution and ethanol. The crude product was recrystallized from 1,4-dioxane to give the title compound.

¹H NMR (DMSO-d₆) δ ppm 12.66 (s, 1H), 11.35 (s, 1H), 8.31 (d, J=9.0 Hz, 1H), 8.25 (s, 1H), 6.68 (d, J=1.2 Hz, 1H), 6.54 (dd, J=1.2, 9.0 Hz, 1H), 3.89 (s, 3H), 3.21-3.10 (m, 4H), 2.50-2.44 (m, 4H), 2.25 (s, 3H).

### Example 5G

### 7-(2,6-dichlorobenzyl)-5-{[2-methoxy-4-(4-methylpiperazin-1-yl)phenyl]amino}pyrido[4,3-d]pyrimidin-4(3H)-one

2,6-Dichlorobenzyl zinc bromide solution in tetrahydrofuran (1 N, 2.3 mL, 2.3 mmol) was added to a solution of bis(triphenylphosphine)palladium(II) chloride (26.7 mg) and EXAMPLE SF (153 mg) in anhydrous tetrahydrofuran (5 mL) and the mixture was stirred under nitrogen at 65° C. for 20 hours. After cooling to ambient temperature, the mixture was diluted with water and extracted with ethyl acetate. The combined organic layers were dried over anhydrous sodium sulfate, filtered, and concentrated in vacuo. The residue was purified by flash chromatography on silica gel (200-300 mesh) using 50/1 dichloromethane/methanol to afford the crude product, which was further purified by recrystallization from methanol to give the title compound. ¹H NMR (DMSO-d₆) δ ppm 12.44 (brs., 1H), 11.22 (brs, 1H), 8.17 (s, 1H), 8.00 (d, J=8.9 Hz, 1H), 7.59 (s, 1H), 7.56 (s, 1H), 7.46-7.38 (m, 1H), 6.60 (s, 2H), 6.18 (dd, J=2.7, 9.7 Hz, 1H), 4.41 (s, 2H), 3.85 (s, 3H), 3.14-3.05 (m, 4H), 2.20-2.43 (m, 4H), 2.24 (s, 3H). MS: 525 (M+H⁺).

### Example 6

### 7-(2,6-dichlorobenzyl)-5-{[2-methoxy-4-(piperazin-1-yl)phenyl]amino}pyrido[4,3-d]pyrimidin-4(3H)-one

### Example 6A

### tert-butyl 4-(4-(7-chloro-3,4-dihydro-4-oxopyrido[4,3-d]pyrimidin-5-yl amino)-3-methoxyphenyl)piperazine-1-carboxy late

The title compound was obtained following the procedure described in EXAMPLE 5F substituting tert-butyl 4-(4-amino-3-methoxyphenyl)piperazine-1-carboxylate for 2-methoxy-4-(4-methylpiperazin-1-yl)aniline. ¹H NMR (DMSO-d₆) δ ppm 11.36 (brs, 1H), 8.34 (d, J=8.9 Hz, 1H), 8.25 (s, 1H), 6.79 (s, 1H), 6.72 (d, j=2.1 Hz, 1H), 6.57 (dd, J=2.1, 8.9 Hz, 1H), 3.89 (s, 3H), 3.51-3.44 (m, 4H), 3.14-3.08 (m, 4H), 1.43 (s, 9H).

### Example 6B

### tert-butyl 4-(4-(7-(2,6-dichlorobenzyl)-3,4-dihydro-4-oxopyrido[4,3-d]pyrimidin-5-ylamino)-3-methoxyphenyl)piperazine-1-carboxy late

The title compound was obtained following the procedure described in EXAMPLE 5G substituting EXAMPLE 6A for EXAMPLE 5F. MS: 611 (M+H⁺).

### Example 6C

### 7-(2,6-dichlorobenzyl)-5-{[2-methoxy-4-(piperazin-1-yl)phenyl]amino}pyrido[4,3-d]pyrimidin-4(3H)-one

Hydrochloric acid (4 mL) was added to EXAMPLE 6B (90 mg, 0.15 mmol) in 1,4-dioxane (15 mL) and methanol (5 mL) and the mixture stirred at ambient temperature for 4 hours. The solvents were removed in vacuo and the residue was dissolved in water, and brought to pH=11 with sodium hydroxide. The mixture was extracted with ethyl acetate and the organic layers were dried with sodium sulfate, filtered and concentrated in vacuo to give the crude product, which was recrystallized from methanol to afford the title compound. ¹H NMR (DMSO-d₆) δ ppm 11.27 (s, 1H), 8.15 (s, 1H), 8.00 (d, J=8.8 Hz, 1H), 7.58 (s, 1H), 7.55 (s, 1H), 7.45-7.46 (m, 1H), 6.58 (s, 2H), 6.20-6.13 (m, 1H), 4.40 (s, 2H), 3.84 (s, 3H), 3.04-2.96 (m, 4H), 2.90-2.81 (m, 4H). MS: 511 (M+H⁺).

### Example 7

### 7-(2,6-dichlorobenzyl)-5-{[4-(piperazin-1-yl)phenyl]amino}pyrido[4,3-d]pyrimidin-4(3H)-one

### Example 7A

### tert-butyl 4-(4-(7-chloro-3,4-dihydro-4-oxopyrido[4,3-d]pyrimidin-5-ylamino) phenyl)piperazine-1-carboxy late

The title compound was obtained following the procedure described in EXAMPLE 5F substituting tert-butyl 4-(4-aminophenyl)piperazine-1-carboxylate for tert-butyl 4-(4-amino-3-methoxyphenyl)piperazine-1-carboxylate. MS: 457 (M+H⁺).

### Example 7B

### tert-butyl 4-(4-(7-(2,6-dichlorobenzyl)-3,4-dihydro-4-oxopyrido[4,3-d]pyrimidin-5-ylamino)phenyl)piperazine-1-carboxylate

The title compound was obtained following the procedure described in EXAMPLE 5G substituting EXAMPLE 7A for EXAMPLE 5F. MS: 581 (M+H⁺).

### Example 7C

### 7-(2,6-dichlorobenzyl)-5-{[4-(piperazin-1-yl)phenyl]amino}pyrido[4,3-d]pyrimidin-4(3H)-one

The title compound was obtained following the procedure described in EXAMPLE 6C substituting EXAMPLE 7B for EXAMPLE 6B. ¹H NMR ((DMSO-d₆) δ ppm 11.11 (s, 1H), 8.16 (s, 1H), 7.58 (s, 1H), 7.55 (s, 1H), 7.45-7.34 (m, 3H), 6.73 (d, J=9.0 Hz, 2H), 6.57 (s, 1H), 4.39 (s, 2H), 3.02-2.95 (m, 4H), 2.91-2.83 (m, 4H). MS: 481 (M+1).

### Example 8

### 6-(2,6-dichlorobenzyl)-8-{[2-methoxy-4-(piperazin-1-yl)phenyl]amino}-2,7-naphthyridin-1(2H)-one

The title compound was obtained following the procedure of EXAMPLE 4 substituting tert-butyl 4-(4-amino-3-methoxyphenyl)piperazine-1-carboxylate for 2-methoxy-4-(4-methylpiperazin-1-yl)aniline. ¹H NMR (DMSO-d₆) δ ppm 11.83 (s, 1H), 11.45 (brs, 1H), 8.27 (s, 1H), 8.05 (d, J=9 Hz, 1H), 7.57 (d, J=8.1 Hz, 2H), 7.42-7.37 (m, 1H), 7.33-7.30 (m, 1H), 6.60 (s, 2H), 6.39 (d, J=7.2 Hz, 1H), 6.18 (dd, J=2.3, 9.0 Hz, 1H), 4.36 (s, 2H), 3.8 (s, 3H), 3.08-3.06 (m, 4H), 2.97-2.95 (m, 4H). MS: 510 (M+1).

### Example 9

### 6-(2,6-dichlorobenzyl)-8-{[4-(piperazin-1-yl)phenyl]amino}-2,7-naphthyridin-1(2H)-one

The title compound was obtained following the procedure of EXAMPLE 4 substituting tert-butyl 4-(4-aminophenyl)piperazine-1-carboxylate for 2-methoxy-4-(4-methylpiperazin-1-yl)aniline. ¹H NMR (DMSO-d₆) δ ppm 11.68 (s, 1H), 8.20 (s, 1H), 7.56-7.50 (m, 2H), 7.41-7.29 (m, 4H), 6.73 (d, J=9.3 Hz, 2H), 6.60 (s, 1H), 6.40 (d, J=6.9 Hz, 1H), 4.35 (s, 2H), 3.06-2.97 (m, 4H), 2.95-2.88 (m, 4H). MS: 480 (M+1).

### Example 10

### 6-(2,6-dichlorobenzyl)-8-({4-[4-(dimethylamino)piperidin-1-yl]-2-methoxyphenyl}amino)-2,7-naphthyridin-1(2H)-one

The title compound was obtained following the procedure of EXAMPLE 4 substituting 1-(4-amino-3-methoxyphenyl)-N,N-dimethylpiperidin-4-amine for 2-methoxy-4-(4-methylpiperazin-1-yl)aniline. ¹H NMR (DMSO-d₆) δ ppm 11.87 (s, 1H), 11.64 (s, 1H), 8.10 (d, 0.1=8.7 Hz, 1H), 7.57 (d, J=8.1 Hz, 2H), 7.42-7.36 (m, 2H), 6.70 (brs, 1H), 6.60 (s, 1H), 6.38 (d, f=6.6 Hz, 1H), 619 (brs, 1H), 4.36 (s. 21-1), 3.85 (s, 3H), 3.79-3.76 (m, 4H), 3.30 (brs, 1H), 2.79 (s, 6H), 2.10-2.08 (m, 2H), 1.77-1.74 (m, 2H). MS: 552 (M+1).

### Example 11

### 7-(2,6-dichlorobenzyl)-5-{[2-methoxy-4-(piperazin-1-yl)phenyl]amino}pyrido[2,3-d]pyrimidin-4(3H)-one

### Example 11A

### 2,6-dichloropyridine 1-oxide

A solution of 2,6-dichloropyridine (4.0 g, 27.0 mmol), 30% hydrogen peroxide (5.2 g, 46.0 mmol) and trifluoroacetic acid (40.0 g) was stirred at 100° C. for 6 hours. The mixture was diluted with water (50 mL) and extracted with ethyl acetate (3×50 mL). The combined organic layers were washed with aqueous sodium bicarbonate and water and concentrated under vacuum to give the title compound, which was used in the next step without further purification.

### Example 1113

### 2,4,6-trichloropyridine

A solution of EXAMPLE 11A (3.8 g, crude) in phosphorus oxychloride was stirred at 100° C. for 6 hours. The mixture was concentrated, quenched with crushed ice and adjusted to pH 8-9 with sodium carbonate. The residue was extracted with ethyl acetate (3×50 mL) and the combined organic layers concentrated under vacuum. The residue was purified by flash chromatography on silica gel (200-300 mesh) eluting with 80/1 petroleum ether/ethylacetate to give the title compound. ¹H NMR (CDCl₃,) δ ppm 7.31 (s, 2H).

### Example 11C

### 2,4,6-trichloronicotinic acid

A solution of diisopropylamine (2.54 g, 22.1 mmol) and n-butyl lithium (1.6 M in hexane, 15.7 mL, 25.1 mmol) in tetrahydrofuran (100 mL) was stirred for 30 minutes at −78° C. A solution of the product of EXAMPLE 11B (2.0 g, 11.0 mmol) in tetrahydrofuran (8 mL) was added dropwise over 30 minutes, followed by stirring for 1 hour. The mixture was poured into dry ice and stirred for 1 hour at room temperature. The mixture was acidified with 10% aqueous hydrochloric acid (20 mL), diluted with an aqueous saturated sodium chloride solution and extracted with ethyl acetate. The organic layer was washed, dried over anhydrous sodium sulfate, filtered, and concentrated under vacuum. The solvent was removed under vacuum to give the crude title compound which was used in the next step without further purification.

### Example 11D

### 2,4,6-trichloronicotinamide

A solution of the product of EXAMPLE 11C (1.5 g, 6.7 mmol) in dichloromethane (50 mL) was treated at room temperature with 2 drops of N,N-dimethylformamide. Oxalyl chloride (1.27 g, 10 mmol) was added dropwise over 15 minutes and stirring was continued for 2 hours. The solution was concentrated and dried under vacuum to give the crude acid chloride. Ammonium (gas) was passed through a solution of the acid chloride in tetrahydrofuran (20 mL) and the mixture stirred at room temperature for 0.5 hours. The mixture was concentrated under vacuum and the residue purified by flash chromatography on silica gel (200-300 mesh) eluting with 100/1 dichloromethane/methanol to give the title compound. MS: 225 (M+H⁺).

### Example 11E

### 2-amino-4,6-dichloronicotinamide

A solution of EXAMPLE 11D in ammonia (10 mL) and 1,4-dioxane was heated at 100° C. in a sealed tube overnight. The mixture was concentrated and purified by flash chromatography on silica gel (200-300 mesh) eluting with 50/1 dichloromethane/methanol to give the title compound. MS: 206 (M+H⁺).

### Example 11F

### 5,7-dichloropyrido[2,3-d]pyrimidin-4(3H)-one

A solution of the product of EXAMPLE 11E (205 mg, 1 mmol) in triethyl orthoformate (30 mL) was heated at 140° C. for 8 hours. The mixture was concentrated under vacuum and the residue purified by flash chromatography on silica gel (200-300 mesh) eluting with 50/1 dichloromethane/methanol to give the title compound. MS: 216 (M+H⁺). ¹H NMR (DMSO-d₆): δ ppm 8.33 (s, 1H), 7.79 (s, 1H).

### Example 11G

### tert-butyl 4-(4-(7-chloro-4-oxo-3,4-dihydropyrido[2,3-d]pyrimidin-5-ylamino)-3-methoxyphenyl)piperazine-1-carboxylate

A solution of the product of EXAMPLE 11F (150 mg, 0.7 mmol), tert-butyl 4-(4-amino-3-methoxy-phenyl)piperazine-1-carboxylate (308 g, 1 mmol) and diisopropylethylamine (774 mg, 6.0 mmol) in 1,4-dioxane (50 mL) was stirred at 100° C. for 48 hours. The mixture was concentrated under vacuum and the residue was purified by flash chromatography on silica gel (200-300 mesh) eluting with 20/1 dichloromethane/methanol to give the title compound. MS: 487 (M+H⁺). ¹H NMR (CDCl₃) δ ppm 8.42 (s, 1H), 7.27 (s, 1H). 6.55 (m, 3H), 3.84 (m, 3H), 3.63 (t, 4H), 3.21 (t, 4H), 1.27 (s, 9H).

### Example 11H

### tert-butyl 4-(4-(7-(2,6-dichlorobenzyl)-4-oxo-3,4-dihydropyrido[2,3-d]pyrimidin-5-ylamino) 3-methoxyphenyl)piperazine-1-carboxylate

A solution of EXAMPLE 11G (200 mg, 0.4 mmol), (2,6-dichlorobenzyl)zinc(II) bromide (1.0 M in tetrahydrofuran, 4.0 mL, 4.0 mmol) and bis(triphenylphosphine)palladium(II) dichloride (28 mg, 0.04 mmol) in tetrahydrofuran (15 mL) was stirred at 70° C. under nitrogen for 16 hours. The mixture was neutralized with 15 mL ammonium chloride solution. The product was extracted with ethyl acetate (3×30 mL) and the combined organic layers were washed with brine, dried over anhydrous sodium sulfate, filtered, and concentrated under vacuum. The residue was purified by flash chromatography on silica gel (200-300 mesh) eluting with 50/1 dichloromethane/methanol and further purified by preparative HPLC (acetonitrile/water containing 0.1% trifluoroacetic acid) to give the title compound. MS: 611 (M+H⁺).

### Example 11I

### 7-(2,6-dichlorobenzyl)-5-{[2-methoxy-4-(piperazin-1-yl)phenyl]amino}pyrido[2,3-d]pyrimidin-4(3H)-one

To a solution of EXAMPLE 11H (35 mg, 0.057 mmol) in methanol (4 mL) at room temperature was added 4 N hydrochloride in 1,4-dioxane (5 mL) and the mixture was stirred at room temperature for 4 hours. The mixture was concentrated and washed with ether. The solid was partitioned between ethyl acetate and saturated sodium bicarbonate solution and the organic layer was dried over anhydrous sodium sulfate, filtered and concentrated. The residue was purified by preparative HPLC (acetonitrile/water containing 0.1% trifluoroacetic acid) to give the title compound. MS: 511 (M+H⁺). ¹H NMR (CD₃OD) δ ppm 8.47 (s, 1H), 7.45 (d, J=7.8 Hz, 2H), 7.34 (m, 1H), 7.08 (d, J=8.4 Hz, 1H), 6.57 (m, 2H), 5.79 (t, 1H), 4.47 (s, 2H), 3.70 (s, 3H), 3.46 (m, 4H), 3.39 (m, 4H).

### Example 12

### 7-(2,6-dichlorobenzyl)-5-{[2-methoxy-4-(piperazin-1-yl)phenyl]amino}pyrimido[4,5-d]pyrimidin-4(3H)-one

### Example 12A

### 2,4,6-trichloropyrimidine-5-carboxylic acid

A solution of diisopropylamine (2.54 g, 22.1 mmol), n-butyl lithium (1.6 M in hexane, 15.7 mL, 25.1 mmol) in tetrahydrofuran (100 mL) was stirred at −78° C. for 30 minutes and a solution of 2,4,6-trichloropyrimidine (2.0 g, 11.0 mmol) in tetrahydrofuran (8 mL) was added dropwise over 30 minutes. After stirring for 1 hour, the mixture was poured into dry ice and the mixture was stirred for 1 hour at room temperature. The mixture was acidified with 10% aqueous hydrochloric acid (20 mL), diluted with aqueous sodium chloride and extracted with ethyl acetate. The organic layer was washed, dried over anhydrous sodium sulfate, filtered, and concentrated under vacuum. The solvent was removed by azeotropic distillation with chloroform and the residue was triturated with hexane to give the title compound, which was used in the next step without further purification. MS: 227 (M+H⁺).

### Example 12B

### 4-amino-2,6-dichloropyrimidine-5-carboxamide

A solution of the product of EXAMPLE 12A (1.1 g, 5.0 mmol) in tetrahydrofuran (30 mL) was treated with 2 drops of N,N-dimethylformamide. Oxalyl chloride (1.0 mL, 10 mmol) was added dropwise over 15 minutes and the mixture was stirred at room temperature for 2 hours. The solution was concentrated and dried under vacuum to give the crude acid chloride. A solution of the acid chloride in tetrahydrofuran (10 mL) was added dropwise to a solution of ammonium hydroxide (5 mL) in tetrahydrofuran at 0° C. After stirring at room temperature for 2 hours, the mixture was concentrated under vacuum. The residue was purified by flash chromatography on silica gel (200-300 mesh) eluting with 50/1 dichloromethane/methanol to give the title compound. MS: 207 (M+H⁺).

### Example 12C

### tert-butyl 4-(4-(6-amino-5-carbamoyl-2-chloropyrimidin-4-ylamino)-3-methoxyphenyl)-piperazine-1-carboxylate

A solution of the product of EXAMPLE 12B (621 mg, 3.0 mmol), tert-butyl 4-(4-amino-3-methoxy-phenyl)piperazine-1-carboxylate (1.1 g, 3.6 mmol) and diisopropylethylamine (774 mg, 6.0 mmol) in 1,4-dioxane (50 mL) was stirred at 100° C. for 48 hours. The mixture was concentrated under vacuum and the residue was purified by flash chromatography on silica gel (200-300 mesh) eluting with 30/1 dichloromethane/methanol to give the title compound. MS: 478 (M+H⁺).

### Example 12D

### tert-butyl 4-(4-(2-chloro-5-oxo-5,6-dihydropyrimido[4,5-d]pyrimidin-4-ylamino)-3-methoxy-phony 1)piperazine-1-carboxy late

A solution of EXAMPLE 12C (250 mg, 0.5 mmol) in triethyl orthoformate (50 mL) was stirred at 140° C. for 12 hours. The mixture was concentrated under vacuum and the residue was recrystallized from methanol to give the title compound. MS: 488 (M+H⁺). NMR (DMSO-d₆): δ ppm 9.89 (s, 1H), 7.96 (d, J=8.7 Hz, 1H), 7.67 (s, 2H), 7.09 (s, 2H), 6.66 (s, 1H), 6.50 (d, J=8.7 Hz, 1H), 3.82 (s, 3H), 3.45 (m, 4H), 3.07 (m, 4H), 1.42 (s, 9H).

### Example 12E

### tert-butyl 4-(4-(2-(2,6-dichlorobenzyl)-5-oxo-5,6-dihydropyrimido[4,5-d]pyrimidin-4-ylamino)-3-methoxyphenyl)piperazine-1-carboxylate

A solution of the product of EXAMPLE 12D (100 mg, 0.2 mmol), (2,6-dichlorobenzyl)zinc(II) bromide (1.0 M in tetrahydrofuran, 2.0 mL. 2.0 mmol) and bis(triphenylphosphine)palladium(II) dichloride (14.3 mg, 0.02 mmol) in tetrahydrofuran (5 mL) was stirred at 70° C. under nitrogen for 8 hours. The mixture was neutralized with 15 mL ammonium chloride solution and the product was extracted with ethyl acetate (3×30 mL). The combined organic layers were washed with brine, dried over anhydrous sodium sulfate, filtered, and concentrated under vacuum. The residue was purified by flash chromatography on silica gel (200-300 mesh) eluting with 30/1 dichloromethane/methanol to give the title compound. ¹H NMR (DMSO-d₆): δ ppm 13.10 (s, 1H), 11.50 (s, 1H), 8.42 (s, 1H), 8.20 (d, =8.7 Hz, 1H), 6.70 (s, 1H), 6.57 (d, J=8.7 Hz, 1H), 3.89 (s, 314), 3.46 (m, 4H), 3.13 (m, 4H), 1.42 (s, 9H). MS: 612 (M+H⁺).

### Example 12F

### 7-(2,6-dichlorobenzyl)-5-{[2-methoxy-4-(piperazin-1-yl)phenyl]amino}pyrimido[4,5-d]pyrimidin-4(3H)-one

To a solution of the product of EXAMPLE 12E (60 mg, 0.1 mmol) in dichloromethane (10 mL) was added _{trifluoroacetic acid} (2 mL). After stirring at room temperature for 8 hours, the mixture was concentrated and purified by preparative HPLC (acetonitrile/water containing 0.1% trifluoroacetic acid) to give the title compound. ¹H NMR (DMSO-d₆): δ ppm 11.50 (s, 1H) 8.79 (s, 2H), 8.42 (s, 1H), 7.75 (d, 1H), 7.58 (d, 2H), 7.43 (t, 1H), 6.68 (s, 1H), 6.17 (d, 1H), 4.48 (s, 2H), 3.88 (s, 3H), 3.30 (m, 4H), 3.26 (m, 4H). MS: 512 (M+H⁺).

### Example 13A

### 2-(2,6-dichlorobenzyl)-4-chloro-1,6-naphthyridin-5(6)-one

To a suspension of 60% sodium hydride (71 mg, 1.76 mmol) in anhydrous N,N-dimethylformamide (2 mL) was added dropwise a solution of EXAMPLE 1F (300 mg, 0.84 mmol) in dry N,N-dimethylformamide (2 mL) at ambient temperature under nitrogen. After stirring at ambient temperature for 15 minutes, 1,3,5-triazine (136 mg, 1.68 mmol) in dry N,N-dimethylformamide (2 mL) was added. The mixture was heated at 100° C. for 18 hours, cooled to ambient temperature, and poured into saturated aqueous ammonium chloride (50 mL). The solution was extracted with ethyl acetate (3×30 mL) and the organic layers were combined, dried over anhydrous sodium sulfate, filtered and concentrated. The residue was purified by preparative thin layer chromatography using 20:1 dichloromethane/methanol to afford the title compound. MS: 341 (M+H⁺).

### Example 13B

### tert-butyl 4-(4-(2-(2,6-dichlorobenzyl)-5,6-dihydro-5-oxo-1,6-naphthyridin-4-ylamino)-2-fluorophenyl)piperazine-1-carboxylate

A solution of the product of EXAMPLE 13A (70 mg, 0.21 mmol), tert-butyl 4-(4-amino-2-fluorophenyl)piperazine-1-carboxylate (61 mg, 0.21 mmol) and catalytic p-toluenesulfonic acid (5 mg) in n-butanol (3 mL) was heated at 100° C. for 18 hours. After cooling to ambient temperature, the mixture was poured into saturated aqueous sodium bicarbonate (50 mL). The resulting solution was extracted with ethyl acetate (3×30 mL), and the organic layers were combined, dried over anhydrous sodium sulfate, filtered and concentrated. The residue was purified by preparative thin layer chromatography using 20:1 dichloromethane/methanol to afford the crude title compound which was used in the next step without further purification. MS: 598 (M+H⁺).

### Example 13C

### 2-(2,6-dichlorobenzyl)-4-(3-fluoro-4-(piperazin-1-yl)phenylamino)-1,6-naphthyridin-5(6H)-one

To a solution of the product of EXAMPLE 13B (73 mg, crude) in dry dichloromethane (5 mL) was added trifluoroacetic acid (5 mL) slowly at ambient temperature and the mixture stirred at ambient temperature for 18 hours. The solvent was removed under vacuum and the residue was purified by preparative HPLC using a gradient of 60/40 to 95/5 acetonitrile/water containing 0.1% trifluoroacetic acid to afford the title compound as a mono trifluoroacetate salt. ¹H NMR (CD₃OD) δ 7.64 (d, J=7.2 Hz, 1H), 7.38-7.32 (m, 2H), 7.23 (dd, J=9.0, 6.9 Hz, 1H), 7.06-6.88 (m, 3H), 6.53 (d, =7.2 Hz, 1H), 5.96 (s, 1H), 4.38 (s, 2H), 3.34-3.28 (m, 4H), 3.26-3.20 (m, 4H). MS: 249.6 (M/2+H⁺).

### Example 14

### 2-(2,6-dichlorobenzyl)-4-{[2-methoxy-4-(piperazin-1-yl)phenyl]amino}pyrido[4,3-d]pyrimidin-5(6H)-one

### Example 14A

### 2-(1-ethoxyethylidene)malononitrile

A mixture of malononitrile (9.1 g, 0.138 mol), triethyl orthoacetate (26.8 g, 0.165 mol) and glacial acidic acid (0.4 mL) was heated to 90° C. and then to 140° C. for 30 minutes. The mixture was cooled to ambient temperature and the resulting solid was washed with ethanol (50 mL) to afford a solid, which was filtered, washed with ethanol and dried under vacuum to provide the title compound.

### Example 14B

### 4-amino-6-methyl-2-(methylthio)pyrimidine-5-carbonitrile

To a mixture of EXAMPLE 14A (25 g, 0.184 mol) and S-methlylisothiourea hemisulfate salt (38.3 g, 0.275 mol) in methanol (700 mL) at 0° C. was added sodium methanolate (9.9 g, 0.184 mol) and the mixture was stirred at ambient temperature overnight. Water (1 L) was added and the stirring was continued for additional 30 minutes. The resulting precipitate was filtered and washed with water until the washes were colorless. The solid was dried under vacuum to give the title compound. MS: 181.1 (M+H⁺).

### Example 14C

### 4-chloro-6-methyl-2-(methylthio)pyrimidine-5-carbonitrile

The product of EXAMPLE 14B (50 g, 0.278 mol) was added to a mixture of anhydrous copper (II) chloride (44.7 g, 0.334 mol) and tert-butylnitrite (51.6 mL, 0.5 mol) in acetonitrile (800 mL) at 80° C. After stirring for 3 hours, the mixture was cooled to room temperature and filtered. The filtrate was concentrated and the residue was dissolved in ethyl acetate, washed with water and brine, dried over sodium sulfate, filtered, and concentrated. The residue was purified by flash chromatography on silica gel eluting with a gradient of 3/1 to 3/2 petroleum ether/ethyl acetate to afford the title compound. MS: 200.1 (M+H⁺).

### Example 14D

### tert-butyl 4-(3-methoxy-4-nitrophenyl)piperazine-1-carboxylate

A suspension of 4-fluoro-2-methoxy-1-nitrobenzene (15 g, 87 mmol), tert-butyl piperazine-1-carboxylate (19.59 g, 105.2 mmol) and potassium carbonate (24 g, 174 mmol) in N,N-dimethylformamide (150 mL) was heated at 80° C. for 8 hours. After cooling to ambient temperature, the mixture was poured in water (500 mL). The precipitate was filtered and washed with ethanol to give the title compound. MS: 338 (M+H⁺).

### Example 14E

### tert-butyl 4-(4-amino-3-methoxyphenyl)piperazine-1-carboxylate

A suspension of EXAMPLE 14D (6.3 g, 18.7 mmol) and Raney nickel (2.0 g) in 300 mL methanol was stirred under hydrogen at ambient temperature for 5 hours. The mixture was filtered through diatomaceous earth and the filtrate was concentrated. The residue was purified by flash chromatography on silica gel (200-300 mesh) eluting with a gradient of 2/1 to 1/1 petroleum/ethyl acetate to give the title compound.

### Example 14F

### tert-butyl 4-(4-(5-cyano-6-methyl-2-(methylthio)pyrimidin-4-ylamino)-3-methoxyphenyl)piperazine-1-carboxylate

To a solution of EXAMPLE 14C (1.032 g, 5.17 mmol) in N,N-dimethylformamide (12 mL) was added EXAMPLE 14E (1.91 g, 6.2 mol) and N,N-diisopropylethylamine (1.47 g, 11.37 mmol), and the mixture was stirred at 70° C. for 9 hours. The cooled mixture was concentrated and the residue was dissolved in ethyl acetate, washed with water (20 mL), dried over sodium sulfate, filtered, and concentrated. The residue was purified by flash chromatography on silica gel (200˜300 mesh) eluting with a gradient of 3/1 to 1/2 hexane/ethyl acetate to give the title compound. MS: 441 (M+H⁺).

### Example 14G

### (E)-tert-butyl 4-(4-(5-cyano-6-(2-(dimethylamino)vinyl)-2-(methylthio)pyrimidin-4-ylamino)-3-methoxyphenyl)piperazine-1-carboxylate

To a solution of EXAMPLE 14F in N,N-dimethylformamide (6 mL) was added N,N-dimethylformamide dimethyl acetal (0.666 g) and the mixture was stirred at 110° C. for 1.5 hours. The mixture was concentrated and the residue was dissolved in ethyl acetate (30 mL). The solution was washed with water, dried over sodium sulfate, filtered and concentrated to give the crude title compound. MS: 496.2 (M+H⁺).

### Example 14H

### 5-bromo-N-(2-methoxy-4-(piperazin-1-yl)phenyl)-2-(methylthio)pyrido[4,3-d]pyrimidin-4-amine

To a solution of EXAMPLE 14G (1.45 g, 2.75 mmol) in acetic acid (10 mL) was added a solution of 45% v/v hydrobromic acid in glacial acetic acid (8.5 mL) and the mixture was stirred at room temperature for 1 hour. The mixture was concentrated to give the crude title compound as a solid hydrobromic acid salt which was used in the next step without any further purification. MS: 431.35 (M+H⁺).

### Example 14I

### 4-(2-methoxy-4-(piperazin-1-yl)phenylamino)-2-(methylthio)pyrido[4,3-d]pyrimidin-5(6H)-one

The crude product of EXAMPLE 14H (1.2 g, 2.60 mmol) was dissolved in acetic acid (7.5 mL) and 6N aqueous hydrochloric acid (11 mL) and the mixture was stirred at 80° C. for 1.5 hours. The residue was treated slowly with saturated aqueous sodium bicarbonate and the solid was collected by filtration. The aqueous solution was extracted with dichloromethane, dried over sodium sulfate, filtered, and concentrated to provide the crude title compound. MS: 369 (M+H⁺).

### Example 14J

### 2-(2,6-dichlorobenzyl)-4-(2-methoxy-4-(piperazin-1-yl)phenylamino)pyrido[4,3-d]pyrimidin-5(6H)-one

To a mixture of EXAMPLE 14I (160 mg, 0.435 mmol) and tris(dibenzylideneacetone)dipalladium (50 mg, 0.0435 mmol) was added N-methyl-2-pyrrolidone (1 mL) and 0.5N (2,6-dichlorophenyl)zinc(II) bromide in tetrahydrofuran (8.7 mL) under nitrogen and the mixture was heated in a Biotage Microwave Synthesizer at 100° C. for 40 minutes. After cooling to room temperature and concentration, the residue was suspended in water and filtered. The solid was dissolved in 2,2,2-trifluoroacetic acid and concentrated. The residue was purified by preparative HPLC using a gradient of 10/90 to 90/10 acetronitrile in water (containing 0.1% trifluoroacetic acid) to give the title compound. ¹H NMR (CD₃CD, 300 MHz): δ 7.85 (m, 1H), 7.65 (m, 1H), 7.55 (m, 2H), 7.43 (dd, J=6.9 Hz, J=9 Hz, 1H), 6.71 (d, J=2.7 Hz, 1H), 6.56 (s, J=7.2 Hz, 1H), 6.22 (dd, J=2.7 Hz, J=9 Hz, 1H), 4.64 (s, 2H), 3.96 (s, 3H), 3.42 (m, 8H). MS: 481 (M+H⁺).

### Example 15

### 2-(2,6-dichlorobenzyl)-4-{[4-(piperazin-1-yl)phenyl]amino}pyrido[4,3-d]pyrimidin-5(6H)-one

### Example 15A

### tert-butyl 4-(4-(5-cyano-6-methyl-2-(methylthio)pyrimidin-4-ylamino)phenyl)piperazine-1-carboxylate

The title compound was obtained following the procedure described in EXAMPLE 14F, using tert-butyl 4-(4-aminophenyl)piperazine-1-carboxylate in place of EXAMPLE 14E. MS: 441 (M+H⁺).

### Example 15B

### (E)-tert-butyl 4-(4-(5-cyano-6-(2-(dimethylamino)vinyl)-2-(methylthio)pyrimidin-4-ylamino)phenyl)piperazine-1-carboxy late

The title compound was obtained following the procedure described in EXAMPLE 14G using EXAMPLE 15A in place of EXAMPLE 14F. MS: 496 (M+H⁺).

### Example 15C

### 5-bromo-2-(methylthio)-N-(4-(piperazin-1-yl)phenyl)pyrido[4,3-d]pyrimidin-4-amine

The title compound was obtained following the procedure described in EXAMPLE 14H, using EXAMPLE 15B in place of EXAMPLE 14G. MS: 369 (M+H⁺).

### Example 15D

### 2-(methylthio)-4-(4-(piperazin-1-yl)phenylamino)pyrido[4,3-d]pyrimidin-5(6H)-one

The title compound was obtained following the procedure described in EXAMPLE 14I, using EXAMPLE 15C in place of EXAMPLE 14H. MS: 431.35 (M+H⁺).

### Example 15E

### 2-(2,6-dichlorobenzyl)-4-(4-(piperazin-1-yl)phenylamino)pyrido[4,3-d]pyrimidin-5(6H)-on

The title compound was obtained following the procedure described in EXAMPLE 14J, using EXAMPLE 15D in place of EXAMPLE 14I. ¹H NMR (CD₃CD, 300 MHz): δ ppm 7.79 (d, J=7.5 Hz, 1H), 7.53 (m, 1H), 7.50 (s, 1H), 7.40 (dd, J=9 Hz, 6.9 Hz, 1H), 7.34 (s, 1H), 7.31 (s, 1H), 6.83 (m, 1H), 6.80 (m, 1H), 6.55. (d, J=7.2 Hz, 1H), 4.60 (s, 2H), 3.39 (br, 8H). MS: 481 (M+H⁺).

### Example 16

### 6-(2-chloro-6-fluorobenzyl)-8-{[4-(piperazin-1-yl)phenyl]amino}-2,7-naphthyridin-1(2H)-one

### Example 16A

### 2,6-dihydroxy-4-methylnicotinonitrile

Potassium hydroxide (14 g, 261 mmol) was added to a solution of 2-cyanoacetamide (20 g, 238 mmol) and ethyl 3-oxobutanoate (30.8 g, 238 mmol) in methanol (600 mL) and the mixture was refluxed for 12 hours. The mixture was poured into water (500 mL) and acidified to pH˜1 using concentrated hydrochloric acid. The suspension was stirred at ambient temperature for 12 hours and the precipitate was filtered, washed with water (500 mL) and dried under vacuum to afford the title compound. ¹H NMR (DMSO-d₆) δ ppm 5.57 (s, 1H), 2.22 (s, 3H).

### Example 16B

### 2,6-dichloro-4-methylnicotinonitrile

The product of EXAMPLE 16A (5 g, 33.3 mmol) and phosphorus oxychloride (9.3 mL, 0.1 mol) were heated in a sealed tube at 160° C. for 7 hours. The mixture was cooled, poured into ice-water and stirred at ambient temperature for 1 hours. The precipitate was filtered, washed with water and dried in vacuo to provide the title compound. ¹H-NMR: (DMSO-d₆) δ ppm 7.81 (s, 1H), 2.53 (s, 3H).

### Example 16C

### (E)-2,6-dichloro-4-(2-(dimethylamino)vinyl)nicotinonitrile

N,N-dimethylformamide dimethyl acetal (0.48 g, 4 mmol) was added dropwise to a solution of EXAMPLE 16B (0.5 g, 2.69 mmol) in N,N-dimethylformamide (10 mL) and the mixture stirred at 100° C. for 2 hours. After cooling to room temperature, the mixture was concentrated in vacuo and the residue purified by flash chromatography on silica gel (200-300 mesh) eluting with dichloromethane to afford the title compound. ¹H-NMR (CDCl₃) δ 7.35 (d, J=13.2 Hz, 1H), 7.05 (s, 1H), 5.26 (d, J=13.2 Hz, 1H), 3.06 (s, 6H).

### Example 16D

### 6,8-dichloro-2,7-naphthyridin-1(2H)-one

Concentrated hydrochloric acid (15 mL) and EXAMPLE 16C (3 g, 12.5 mmol) were heated in a sealed tube at 45° C. overnight. Ice (10 g) was added and the precipitate was filtered, washed with cold water and dried in vacuo to give the title compound. ¹H NMR (DMSO-d₆) δ ppm 11.76 (s, 1H), 7.76 (s, 1H), 7.52 (d, J=6.9 Hz, 1H), 6.53 (d, 0.1=6.9 Hz, 1H).

### Example 16E

### tert-butyl 4-(4-(3-chloro-8-oxo-7,8-dihydro-2,7-naphthyridin-1-ylamino)phenyl)piperazine-1-carboxylate

To a solution of EXAMPLE 16D (1.67 g, 7.8 mmol) in dioxane (20 mL) was added tert-butyl 4-(4-aminophenyl)piperazine-1-carboxylate (2.38 g, 8.58 mmol) and N-ethyl-N-isopropylpropan-2-amine (5.03 g, 39 mmol) and the mixture was stirred at 120° C. for 4 hours, cooled to ambient temperature and concentrated. The residue was washed with methanol (10 mL) and ethyl acetate (10 mL), and filtered to give the title compound. MS: 456 (M+H⁺).

### Example 16F

### (2-chloro-6-fluorobenzyl)zinc(II) bromide

To a suspension of zinc powder (322 mg, 4.95 mmol) in tetrahydrofuran (3 mL) at 65° C. under nitrogen was added 1,2-dibromoethane (9 mg, 0.05 mmol) and trimethylsilylchloride (10 mg, 0.09 mmol), and the mixture was stirred at 65° C. for 30 minutes. A solution of 2-chloro-6-fluorobenzyl bromide (1 g, 4.50 mmol) in tetrahydrofuran (10 mL) was added dropwise and the mixture was stirred with heating at 65° C. for 3 hours. The mixture was cooled to ambient temperature to give a solution of (2-chloro-6-fluorobenzyl)zinc(II) bromide in tetrahydrofuran (about 0.5 M).

### Example 16G

### tert-butyl 4-(4-(3-(2-chloro-6-fluorobenzyl)-8-oxo-7,8-dihydro-2,7-naphthyridin-1-ylamino)phenyl)piperazine-1-carboxylate

A suspension of EXAMPLE 16E (150 mg, 0.33 mmol), EXAMPLE 16F (6.6 mL, 3.3 mmol) and tetrakis(triphenylphosphine)palladium(O) (38 mg, 0.03 mmol) in tetrahydrofuran (20 mL) was heated in a sealed tube at 100° C. under nitrogen for 18 hours. After cooling, the mixture was washed with saturated ammonium chloride solution (30 mL) and extracted with ethyl acetate (3×20 mL). The organic layers were dried over anhydrous sodium sulfate, filtered, and concentrated to give a solid which was purified by flash chromatography on silica gel (200-300 mesh) eluting with 30/1 dichloromethane/methanol to give the title compound. MS: 564 (M+H⁺).

### Example 16H

### 6-(2-chloro-6-fluorobenzyl)-8-{[4-(piperazin-1-yl)phenyl]amino-2,7-naphthyridin-1(2H)-one

To a solution of EXAMPLE 16G (150 mg, 0.27 mmol) in dichloromethane (10 mL) was added trifluoroacetic acid (2 mL) and the mixture was stirred at ambient temperature for 2 hours. After concentration, the residue was purified by preparative HPLC eluting with a gradient of 10% to 90% acetonitrile/water (containing 0.1% trifluoroacetic acid) to give the title compound. ¹H NMR (CD₃OD, 300 MHz): δ 7.52 (d, J=8.7, 2 H), 7.34 (m, 3H), 7.18 (m, 1H), 7.00 (d, J=8.7, 2 H), 6.53 (s, 1H), 6.46 (d/=6.9, 2 H), 4.27 (s, 2H), 3.40 (s, 8H). MS: 464 (M+H⁺).

### Example 17

### 6-(2,6-dichlorobenzyl)-8-({2-methoxy-4-[(4-methylpiperazin-1-yl)carbonyl]phenyl}amino)-2,7-naphthyridin-1(2H)-one

### Example 17A

### (3-methoxy-4-nitrophenyl)(4-methylpiperazin-1-yl)methanone

A solution of 3-methoxy-4-nitrobenzoic acid (6.81 g, 34.54 mmol) in sulfurous dichloride (50 mL) was stirred at reflux for 8 hours. After cooling to ambient temperature, the mixture was concentrated and the residue was dissolved in dichloromethane (60 mL). 1-Methylpiperazine (3.6 g, 36.27 mmol) was added at 0° C. and the mixture was stirred at ambient temperature for 4 hours. The mixture was poured into water (100 mL) and was extracted with dichloromethane (2×200 mL). The organic phase was dried over anhydrous sodium sulfate, filtered and concentrated to provide the crude title compound. MS: 280.2 (M+H⁺).

### Example 17B

### (4-amino-3-methoxyphenyl)(4-methylpiperazin-1-yl)methanone

To a solution of EXAMPLE 17A (9 g, 32 mmol) in methanol (100 mL) was added 10% palladium on carbon (1 g) and the mixture was stirred at ambient temperature under hydrogen for 8 hours. The mixture was filtered and the filtrate was concentrated to provide the title compound. MS: 250.2 (M+H⁺).

### Example 17C

### 6-chloro-8-(2-methoxy-4-(4-methylpiperazine-1-carbonyl)phenylamino)-2,7-naphthyridin-1(2H)-one

A mixture of EXAMPLE 16D (300 mg, 1.4 mmol), EXAMPLE 17B (453 mg, 1.82 mmol), and N,N-diisopropylethylamine (903 mg, 7 mmol) in dioxane (20 mL) was heated in sealed tube at 120° C. overnight. After concentration, the residue was purified by flash chromatography on silica gel eluting with 10:1 dichloromethane:methanol to give the title compound. MS: 428 (M+H⁺).

### Example 17D

### 6-(2,6-dichlorobenzyl)-8-(2-methoxy-4-(4-methylpiperazine-1-carbonyl)phenyl amino)-2,7-naphthyridin-1(2H)-one

To a solution of EXAMPLE 17C (100 mg, 0.233 mmol) in tetrahydrofuran (5 mL) was added 0.5M (2,6-dichlorobenzyl)zinc(II) bromide in tetrahydrofuran (5 mL, 2.33 mmol) and tetrakis(triphenylphosphine)palladium (27 mg, 0.023 mmol) and the mixture was heated at 110° C. in a Biotage Microwave Synthesizer for 1 hour. After cooling to ambient temperature, the mixture was filtered and the filtrate was concentrated. The residue was purified by preparative-HPLC eluting with a gradient of 10/90 to 80/20 acetonitrile/water (containing 0.1% trifluoroacetic acid) to give the title compound. ¹H NMR (CD₃OD, 300 MHz): δ ppm 8.42 (d, J=8.4 Hz, 1H), 8.36 (s, 1H), 7.48 (d, J=8.1 Hz, 2H), 7.35-7.26 (m, 2H), 7.03 (d, J=1.8 Hz, 1H), 6.79-6.74 (m, 2H), 6.46 (d, J=7.2 Hz, 2H), 4.52 (s, 2H), 3.98 (s, 3H), 3.83-3.68 (m, 4H), 2.83-2.72 (m, 4H), 2.55 (s, 3H). MS: 552, 554 (M+H⁺).

### Example 18

### 4-{[2-(2,6-dichlorobenzyl)-5-oxo-5,6-dihydro-1,6-naphthyridin-4-yl]amino}-3-methoxy-N-[3-(morpholin-4-yl)propyl]benzamide

### Example 18A

### (3-methoxy-4-nitrophenyl)methanol

To a suspension of sodium borohydride (1.93 g, 50.7 mmol) in tetrahydrofuran (20 mL) was added slowly a solution of 3-methoxy-4-nitrobenzoic acid (5.0 g, 25.4 mmol) in tetrahydrofuran (40 mL). Boron trifluoride etherate (10.7 g, 63.4 mmol) was added dropwise at 0° C. and the mixture was stirred at ambient temperature for 16 hours. The mixture was quenched with saturated ammonium chloride (20 mL), diluted with water (50 mL) and extracted with ethyl acetate (3×100 mL). The combined organic phase was dried over anhydrous sodium sulfate, filtered and concentrated to give the crude title compound.

### Example 18B

### (4-amino-3-methoxyphenyl)methanol

To a solution of EXAMPLE 18A (4.6 g, 25 mmol) in methanol (150 mL) was added 10% Raney Nickel (6.0 mg). After stirring at ambient temperature for 16 hours under hydrogen, the mixture was filtered and concentrated to give the crude title compound as a solid. MS: 154 (M+H⁺).

### Example 18C

### ethyl 4,6-dihydroxy-2-methylnicotinate

A mixture of 2,4,6-trichlorophenol (175 g, 886 mmol), malonic acid (57.6 g, 554 mmol) and phosphorus oxychloride (160 mL, 1.77 mol) was heated at 100° C. for 3 hours. After cooling to ambient temperature and concentration, the residue was poured into ice-water and filtered. The solid was washed with water and dried under vacuum. The solid was suspended in bromobenzene (400 mL) and ethyl 3-aminocrotonate (62.9 g, 487 mmol) was added. The mixture was heated at 155° C. for 1.5 hours, concentrated and washed with 2/1 petroleum ether/ethyl acetate to give the title compound. MS: 198 (M+H⁺).

### Example 18D

### ethyl 4,6-dichloro-2-methylnicotinate

A mixture of EXAMPLE 18C (87.3 g, 443 mmol) and phosphorus oxychloride (300 mL) was heated at 140° C. for 2.5 hours. After cooling to ambient temperature and concentration, the residue was poured into ice-water and extracted with ethyl acetate (300 mL×2). The organic phase was dried over sodium sulfate, filtered, concentrated and purified by silica gel (200-300 mesh) eluting with a gradient of 100/1 to 20/1 petroleum ether/ethyl acetate to give the title compound. MS: 234 (M+H⁺).

### Example 18E

### 2,4-dichloro-1,6-naphthyridin-5(6H)-one

To a suspension of 60% sodium hydride in mineral oil (3.72 g, 101 mmol) in N,N-dimethylformamide (10 mL) and toluene (150 mL) was added dropwise a solution of EXAMPLE 18D (18.0 g, 77.3 mmol) in N,N-dimethylformamide (1 mL) at ambient temperature under nitrogen. After stirring at ambient temperature for 30 minutes, 1,3,5-triazine (9.0 g, 111.1 mmol) in N,N-dimethylformamide (10 mL) was added and the mixture was heated at 100° C. for 8 hours. After cooling to ambient temperature, the mixture was poured into saturated aqueous ammonium chloride (150 mL) and extracted with ethyl acetate (3×300 mL). The organic layers were dried over anhydrous sodium sulfate, filtered and concentrated. The residue was purified by silica gel chromatography eluting with a gradient of 100/1 to 50/1 dichloromethane/methanol to afford the title compound. MS: 214 (M+H⁺).

### Example 18F

### 2-chloro-4-(4-(hydroxymethyl)-2-methoxyphenylamino)-1,6-naphthyridin-5(6H)-one

A solution of EXAMPLE 18E (1.2 g, 5.58 mmol), EXAMPLE 18B (1.02 g, 6.07 mmol), N,N-diisopropylethylamine (2 mL) in dioxane (30 mL) was stirred at 120° C. for 16 hours. The mixture was cooled to ambient temperature and concentrated. The residue was diluted with water (50 mL) and extracted with ethyl acetate (3×100 mL). The combined organic extracts were dried over anhydrous sodium sulfate, filtered and purified by flash chromatography on silica gel (200-300 mesh) eluting with a gradient of 50/1 to 10/1 dichloromethane/methanol to give the title compound. MS: 332 (M+H⁺).

### Example 18G

### 2-(2,6-dichlorobenzyl)-4-(4-(hydroxymethyl)-2-methoxyphenylamino)-1,6-naphthyridin-5(6H)-one

A mixture of EXAMPLE 18F (500 mg, 1.51 mmol), 1M (2,6-dichlorobenzyl)zinc(II) bromide in tetrahydrofuran (15.0 mL, 15.0 mmol), tetrakis(triphenylphosphine)palladium (173 mg, 0.15 mmol) in tetrahydrofuran (10 mL) was heated in a Biotage Microwave Synthesizer at 120° C. for 1 hour. After cooling to ambient temperature, the mixture was filtered and purified by flash chromatography on silica gel (200-300 mesh) eluting with a gradient of 50/1 to 10/1 dichloromethane/methanol to provide the title compound. MS: 456 (M+H⁺).

### Example 18H

### 4-(2-(2,6-dichlorobenzyl)-5-oxo-5,6-dihydro-1,6-naphthyridin-4-ylamino)-3-methoxybenzoic acid

To a suspension of EXAMPLE 18G (310 mg, 0.68 mmol) in acetone (25 mL) at 0° C. Was added dropwise Jones Reagent (2.6M, 0.8 mL) and the mixture was stirred at ambient temperature for 3 hours. The reaction was quenched with isopropyl alcohol (20 mL), filtered and concentrated to give the crude title compound which was used without further purification. MS: 470 (M+H⁺).

### Example 18I

### 4-(2-(2,6-dichlorobenzyl)-5-oxo-5,6-dihydro-1,6-naphthyridin-4-ylamino)-3-methoxy-N-(3-morpholinopropyl)benzamide

To a solution of EXAMPLE 18H (80 mg, 0.17 mmol) in dichloromethane (10 mL) was added 0-(7-azabenzotriazol-1-yl)-N,N,N′,N′-tetramethyluronium hexafluorophosphate (71 mg, 0.18 mmol) and N,N-diisopropylethylamine (0.15 mL). After stirring at ambient temperature for 30 minutes, 3-morpholinopropan-1-amine (122 mg, 0.85 mmol) was added and the mixture was stirred at ambient temperature for 4 hours. The mixture was diluted with water (10 mL) and extracted with dichloromethane (3×10 mL). The combined organic extracts were dried over anhydrous sodium sulfate, filtered and concentrated and the residue was purified by preparative HPLC using a gradient of 10/90 to 90/10 acetronitrile in water (containing 0.1% trifluoroacetic acid) to give the title compound. ¹H NMR (CD₃OD, 300 MHz): δ ppm 7.77 (d, J=7.2 Hz, 1H), 7.56-7.46 (m, 4H), 7.38-7.30 (m, 2H), 6.67 (d, J=7.2 Hz, 1H), 6.03 (s, 1H), 4.52 (s, 2H), 4.06 (br, 2H), 3.83 (br, 5H), 3.55-3.32 (m, 4H), 3.26-3.17 (m, 4H), 2.20-2.05 (m, 2H). MS: 596 (M+Fr).

### Example 19

### 4-{[3-(2,6-dichlorobenzyl)-8-oxo-7,8-dihydro-2,7-naphthyridin-1-yl]amino}-3-methoxy-N-[3-(morpholin-4-yl)propyl]benzamide

### Example 19A

### 6-chloro-8-(4-(hydroxymethyl)-2-methoxyphenylamino)-2,7-naphthyridin-1(21)-one

The title compound was obtained following the procedure described in EXAMPLE 16E, using EXAMPLE 18B in place of tert-butyl 4-(4-aminophenyl)piperazine-1-carboxylate. ¹H NMR (DMSO-d₆, 300 MHz): δ 12.15 (s, 1H), 11.75 (s, 1H), 8.49 (d. J=8.1 Hz, 1H), 7.43 (d, J=6.3 Hz, 1H), 7.03 (d, J=1.5 Hz, 1H), 6.91-6.89 (m, 2H), 6.45 (d, J=7.2 Hz, 1H), 5.14 (t, J=5.7 Hz, 1H), 4.47 (d, J=5.7 Hz, 2H), 3.89 (s, 3H). MS: 332 (M+H⁺).

### Example 19B

### 6-(2,6-dichlorobenzyl)-8-(4-(hydroxymethyl)-2-methoxyphenylamino)-2,7-naphthyridin-1(2H)-one

To a solution of EXAMPLE 19A (430 mg, 1.30 mmol) in tetrahydrofuran (10 mL) was added 1M (2,6-dichlorobenzyl)zinc(II) bromide in tetrahydrofuran (13 mL, 13 mmol) and tetrakis(triphenylphosphine)palladium (150 mg, 0.13 mmol) under nitrogen, and the mixture was heated at 120° C. in a Biotage Microwave Synthesizer for 1 hour. After cooling to ambient temperature, the mixture was poured into brine (30 mL) and extracted with tetrahydrofuran (2×50 mL). The combined organic layers were dried over anhydrous sodium sulfate, filtered, and concentrated and the residue was purified by flash chromatography on silica gel eluting with 50:1 dichloromethane/methanol to give the crude title compound which was further purified by preparative HPLC using a gradient of 10/90 to 90/10 acetronitrile in water (containing 0.1% trifluoracetic acid) to afford the title compound. MS 456 (M+H⁺).

### Example 19C

### 4-(3-(2,6-dichlorobenzyl)-8-oxo-7,8-dihydro-2,7-naphthyridin-1-ylamino)-3-methoxybenzoic acid

To a solution of EXAMPLE 19B (140 mg, 0.31 mmol) in acetone (10 mL) at 0° C. was added Jones reagent (0.48 mL, 1.24 mmol) and the mixture was stirred at ambient temperature for 3 hours. The reaction was quenched by addition of 2-propanol, the insoluble material was filtered off, and the filtrate was concentrated to give the crude title compound. MS 469 (M+H⁺),

### Example 19D

### 4-{[3-(2,6-dichlorobenzyl)-8-oxo-7,8-dihydro-2,7-naphthyridin-1-yl]amino}-3-methoxy-N-[3-(morpholin-4-yl)propyl]benzamide

To a solution of EXAMPLE 19C (70 mg, 0.15 mmol) in dichloromethane (10 mL) were added 3-morpholinopropan-1-amine (65 mg, 0.78 mmol), 2-(7-aza-1H-benzotriazole-1-yl)-1,1,3,3-tetramethyluronium (85 mg, 0.312 mmol) and triethylamine (45 mg, 0.45 mmol). After stirring at ambient temperature for 2 hours, the mixture was poured into water (30 mL) and extracted with dichloromethane (30 mL). The combined organic layers were dried over anhydrous sodium sulfate, filtered, and concentrated and the residue purified by preparative HPLC eluting with a gradient of 10/90 to 80/20 acetonitrile/water (containing 0.1% trifluoroacetic acid) to give the title compound. ¹H NMR (DMSO-d₆, 300 MHz): δ 12.30 (s, 1H), 11.58 (d, J=5.7 Hz, 1H), 9.68 (s, 1H), 8.53-8.50 (m, 1H), 8.35 (d, J=8.4 Hz, 1H), 7.58 (d, J=7.8 Hz, 2H), 7.46-7.38 (m, 3H), 7.23 (d, J=9.3 Hz, 1H), 6.85 (s, 1H), 6.47 (d, J=6.9 Hz, 1H), 4.40 (s, 2H), 4.01-4.93 (m, 2H), 3.71-3.67 (m, 2H), 3.47-3.40 (m, 2H), 3.15-3.13 (m, 4H), 1.96-1.91 (m, 2H). MS: 596, 598 (M+H⁺).

### Example 20

### 2-(2,6-dichlorobenzyl)-4-({2-methoxy-4-[(4-methylpiperazin-1-yl)carbonyl]phenyl}amino)-1,6-naphthyridin-5(6H)-one

The title compound was obtained following the procedure described in EXAMPLE 18I, using 1-methylpiperazine in place of 3-morpholinopropan-1-amine. ¹H NMR (CD₃OD, 300 MHz): δ ppm 7.77 (d, J=7.2 Hz, 1H), 7.49-7.32 (m, 4H), 7.19 (s, 1H), 7.07 (dd, J=7.8 Hz, 1.5 Hz, 1H), 6.66 (d, J=7.2 Hz, 1H), 6.00 (s, 1H), 4.51 (s, 2H), 3.82 (s, 3H), 3.48-3.32 (brs, 8H), 2.98 (s, 3H). MS: 552 (M+H⁺).

### Example 21

### 4-{[2-(2,6-dichlorobenzyl)-5-oxo-5,6-dihydro-1,6-naphthyridin-4-yl]amino}-3-methoxy-N-[2-(pyrrolidin-1-yl)ethyl]benzamide

The title compound was obtained following the procedure described in EXAMPLE 18I, using 2-(pyrrolidin-1-yl)ethanamine in place of 3-morpholinopropan-1-amine. ¹H NMR (CD₃OD, 300 MHz): (δ ppm 7.77 (d, J=7.5 Hz, 1H), 7.58 (d, J=1.5 Hz, 1H), 7.50-7.46 (m, 3H), 7.38-7.32 (m, 2H), 6.66 (d, J=7.5 Hz, 1H), 6.03 (s, 1H), 4.52 (s, 2H), 3.83 (s, 3H), 3.80-3.76 (m, 4H), 3.47 (t, J=11.4 Hz, 2H), 3.23-3.18 (m, 2H), 2.24-2.18 (m, 2H), 2.09-2.04 (m, 2H). MS: 566 (M+H).

### Example 22

### 4-{[3-(2,6-dichlorobenzyl)-8-oxo-7,8-dihydro-2,7-naphthyridin-1-yl]amino}-3-methoxy-N-[2-(pyrrolidin-1-yl)ethyl]benzamide

To a solution of EXAMPLE 26B (70 mg, 0.15 mmol) in dichloromethane (10 mL) was added 2-(pyrrolidin-1-yl)ethanamine (51.3 mg, 0.45 mmol), 2-(7-aza-1H-benzotriazole-1-yl)-1,1,3,3-tetramethyluronium (85 mg, 0.22 mmol) and triethylamine (45 mg, 0.45 mmol). After stirring at ambient temperature for 2 hours, the mixture was poured into water (30 mL) and extracted with dichloromethane (30 mL). The combined organic layers were dried over anhydrous sodium sulfate, filtered, and concentrated and the residue purified by preparative HPLC eluting with a gradient of 10/90 to 80/20 acetonitrile/water (containing 0.1% trifluoroacetic acid) to give the title compound. ¹H NMR (DMSO-d₆, 300 MHz): δ ppm 12.27 (s, 1H), 11.58 (brs, 1H). 8.32-8.27 (m, 2H), 7.57 (d, J=8.4 Hz, 2H), 7.40-7.20 (m, 3H), 7.46-7.38 (m, 3H), 7.20 (d, J=9.0 Hz, 1H), 6.83 (s, 1H), 6.46 (d, J=7.2 Hz, 1H), 4.43 (s, 2H), 3.91 (s, 3H), 2.64-2.59 (m, 2H), 2.56-2.52 (m, 4H), 1.72-1.68 (m, 4H), 1.23 (m, 2H). MS: 566, 568 (M+H⁺).

### Example 23

### 4-{[2-(2,6-dichlorobenzyl)-5-oxo-5,6-dihydro-1,6-naphthyridin-4-yl]amino}-N-[2-(dimethylamino)ethyl]-3-methoxybenzamide

The title compound was obtained following the procedure described in EXAMPLE 18I, using N¹,N¹-dimethylethane-1,2-diamine in place of 3-morpholinopropan-1-amine. ¹H NMR (CD₃OD, 300 MHz): δ ppm 7.76 (d, J=7.2 Hz, 1H), 7.58 (d, J=1.5 Hz, 1H), 7.49-7.45 (m, 3H), 7.37-7.31 (m, 2H), 6.66 (d, J=7.5 Hz, 1H), 6.06 (s, 1H), 4.51 (s, 2H), 3.80 (s, 3H), 3.78 (t, J=11.4 Hz, 2H), =11.4 Hz, 2H), 3.01 (s, 6H). MS: 540 (M+H⁺).

### Example 24

### 4-{[2-(2,6-dichlorobenzyl)-5-oxo-5,6-dihydro-1,6-naphthyridin-4-yl]amino}-3-methoxy-N-[2-(piperidin-1-yl)ethyl]benzamide

The title compound was obtained following the procedure described in EXAMPLE 18I, using 2-(piperidin-1-yl)ethanamine in place of 3-morpholinopropan-1-amine. ¹H NMR (CD₃OD, 300 MHz): δ ppm 7.76 (d, J=7.2 Hz, 1H), 7.58 (d, 1=1.5 Hz, 1H), 7.49-7.46 (m, 3H), 7.37-7.32 (m, 2H), 6.66 (d, J=7.2 Hz, 1H), 6.05 (s, 1H), 4.52 (s, 2H), 3.83 (s, 3H), 3.81-3.70 (m, 4H), 3.37-3.32 (m, 2H), 3.06-2.97 (m, 2H), 2.04-2.80 (m, 6H), MS: 580 (M+H⁺).

### Example 25

### 4-{[2-(2,6-dichlorobenzyl)-5-oxo-5,6-dihydro-1,6-naphthyridin-4-yl]amino}-3-methoxy-N-[2-(4-methylpiperazin-1-yl)ethyl]benzamide

The title compound was obtained following the procedure described in EXAMPLE 18I, using 2-(4-methylpiperazin-1-yl)ethanamine in place of 3-morpholinopropan-1-amine. ¹H NMR (CD₃OD, 300 MHz): δ ppm 7.77 (d, J=7.2 Hz, 1H), 7.53-7.29 (m, 6H), 6.65 (d, J=7.2 Hz, 1H), 6.00 (s, 1H), 4.51 (s, 2H), 3.82 (s, 3H), 3.61-3.56 (m, 4H), 3.32 (brs, 4H), 2.88 (s, 3H), 2.78 (brs, 4H). MS: 595 (M+H⁺).

### Example 26

### 4-{[3-(2,6-dichlorobenzyl)-8-oxo-7,8-dihydro-2,7-naphthyridin-1-yl]amino}-3-methoxy-N-[2-(4-methylpiperazin-1-yl)ethyl]benzamide

### Example 26A

### 6-(2,6-dichlorobenzyl)-8-(4-(hydroxymethyl)-2-methoxyphenylamino)-2,7-naphthyridin-1(2H)-one

To a solution of EXAMPLE 19A (430 mg, 1.30 mmol) in tetrahydrofuran (10 mL) was added 1M (2,6-dichlorobenzyl)zinc(II) bromide in tetrahydrofuran (13 mL, 13 mmol) and tetrakis(triphenylphosphine)palladium (150 mg, 0.13 mmol) under nitrogen, and the mixture was heated at 120° C. in a Biotage Microwave Synthesizer for 1 hour. The mixture was cooled to ambient temperature, poured into brine (30 mL) and extracted with tetrahydrofuran (2×50 mL). The organic layer was dried over anhydrous sodium sulfate, filtered, concentrated and purified by flash chromatography on silica gel eluting with 50:1 dichloromethane/methanol to give the title compound, which was further purified by preparative HPLC using a gradient of 10/90 to 90/10 acetronitrile in water (containing 0.1% trifluoroacetic acid) to give the title compound. MS: 456 (M+H⁺).

### Example 26B

### 4-(3-(2,6-dichlorobenzyl)-8-oxo-7,8-dihydro-2,7-naphthyridin-1-ylamino)-3-methoxybenzoic acid

To a solution of EXAMPLE 26A (140 mg, 0.31 mmol) in acetone (10 mL) at 0° C. was added Jones reagent (0.48 mL, 1.24 mmol) and the mixture was stirred at ambient temperature for 3 hours. The reaction was quenched with addition of 2-propanol, the insoluble material was filtered off and the filtrate was concentrated to give the crude title compound. MS 469 (M+

### Example 26C

### 4-{[3-(2,6-dichlorobenzyl)-8-oxo-7,8-dihydro-2,7-naphthyridin-1-yl]amino}-3-methoxy-N-[2-(4-methylpiperazin-1-yl)ethyl]benzamide

To a solution of EXAMPLE 26B (74 mg, 0.15 mmol) in dichloromethane (10 mL) was added 2-(4-methylpiperazin-1-yl)ethanamine (111 mg, 0.78 mmol), 2-(7-aza-1H-benzotriazole-1-yl)-1,1,3,3-tetramethyluronium (118 mg, 0.312 mmol) and triethylamine (79 mg, 0.78 mmol). After stirring at ambient temperature for 2 hours, the mixture was poured into water (30 mL) and extracted with dichloromethane (30 mL). The combined organic layers were dried over anhydrous sodium sulfate, filtered, concentrated and purified by preparative HPLC using a gradient of 10/90 to 90/H) acetronitrile in water (containing 0.1% trifluoroacetic acid) to give the title compound. ¹H NMR (DMSO-d₆, 300 MHz): δ ppm 12.29 (s, 1H), 11.60 (s, 1H), 8.35-8.31 (m, 2H), 7.58 (d, J=8.1 Hz, 2H), 7.42-7.38 (m, 3H), 7.20 (d, J=8.7 Hz, 1H), 6.84 (s, 1H), 6.47 (d, J=7.5 Hz, 1H), 4.44 (s, 2H), 3.93 (s, 3H), 3.00 (s, 3H), 2.55 (brs, 12H). MS 595.2 (M+H⁺).

### Example 27

### 4-{[3-(2,6-dichlorobenzyl)-8-oxo-7,8-dihydro-2,7-naphthyridin-1-yl]amino}-3-methoxy-N-[2-(piperidin-1-yl)ethyl]benzamide

To a solution of EXAMPLE 26B (60 mg, 0.13 mmol) in dichloromethane (10 mL) were added 2-(piperidin-1-yl)ethanamine (32 mg, 0.25 mmol), 2-(7-aza-1H-benzotriazole-1-yl)-1,1,3,3-tetramethyluronium (73 mg, 0.19 mmol) and N,N-diisopropylethylamine (49.5 mg, 0.38 mmol). After stirring at ambient temperature for 2 hours, the mixture was poured into water (30 mL) and extracted with dichloromethane (30 mL). The combined organic layers were dried over anhydrous sodium sulfate, filtered, concentrated and purified by preparative HPLC eluting with a gradient of 10/90 to 80/20 acetonitrile/water (containing 0.1% trifluoroacetic acid) to give the title compound. ¹H NMR (DMSO-d₆ 300 MHz): δ 12.29 (s, 1H), 5 ppm 11.61 (bs, 1H), 8.36-8.29 (m, 2H), 7.58 (d, J=8.4 Hz, 2H), 7.44-7.39 (m, 3H), 7.18 (d, J=0.9 Hz, 1H), 6.84 (s, 1H), 6.47 (d, J=7.2 Hz, 1H), 4.44 (s, 2H), 3.91 (s, 3H), 3.44-3.42 (m, 2H), 2.61-2.51 (m, 6H), 1.60-1.52 (m, 4H), 1.44-1.41 (m, 2H). MS: 580, 582 (M+H⁺).

### Example 28

### 4-{[3-(2,6-dichlorobenzyl)-8-oxo-7,8-dihydro-2,7-naphthyridin-1-yl]amino}-N-[4-(dimethylamino)butyl]-3-methoxybenzamide

To a solution of EXAMPLE 26B (60 mg, 0.13 mmol) in dichloromethane (10 mL) was added N¹,N¹-dimethylbutane-1,4-diamine (29.7 mg, 0.25 mmol), 2-(7-aza-1H-benzotriazole-1-yl)-1,1,3,3-tetramethyluronium (73 mg, 0.19 mmol) and N,N-diisopropylethylamine (49.5 mg, 0.38 mmol). After stirring at ambient temperature for 2 hours, the mixture was poured into water (30 mL) and extracted with dichloromethane (30 mL). The combined organic layers were dried over anhydrous sodium sulfate, filtered, concentrated and purified by preparative HPLC eluting with a gradient of 10/90 to 80/20 acetonitrile/water (containing 0.1% trifluoroacetic acid) to give the title compound. ¹H NMR (DMSO-d₆, 300 MHz): δ 12.15 (s, 1H), 8.46 (d, J=8.7 Hz, 1H), 7.50-7.23 (m, 6H), 6.75 (s, 1H), 6.47 (d, J=5.1 Hz, 1H), 4.53 (s, 2H), 4.01 (s, 3H), 3.46-3.42 (m, 2H), 3.16-3.14 (m, 6H), 2.87 (s, 6H). MS: 568, 570 (M+H^{÷}).

### Example 29

### 2-(2,6-dichlorobenzyl)-4-{[2-methoxy-4-(piperazin-1-ylcarbonyl)phenyl]amino}-1,6-naphthyridin-5(6H)-one

### Example 29A

### tert-butyl 4-(4-(2-(2,6-dichlorobenzyl)-5-oxo-5,6-dihydro-1,6-naphthyridin-4-ylamino)-3-methoxybenzoyl)piperazine-1-carboxy late

The title compound was obtained following the procedure described in EXAMPLE 18I, using tert-butyl piperazine-1-carboxylate in place of 3-morpholinopropan-1-amine. MS: 638 (M+H⁺).

### Example 29B

### 2-(2,6-dichlorobenzyl)-4-{[2-methoxy-4-(piperazin-1-ylcarbonyl)phenyl]amino}-1,6-naphthyridin-5(6H)-one

To a solution of EXAMPLE 29A (110 mg, 0.17 mmol) in dichloromethane (20 mL) was added trifluoroacetic acid (5 mL) and the mixture was stirred at ambient temperature for 16 hours. After concentration, the residue was purified by preparative HPLC using a gradient of 10/90 to 90/10 acetronitrile in water (containing 0.1% trifluoroacetic acid) to give the title compound. ¹H NMR (CD₃OD, 300 MHz): δ 7.50-7.44 (m, 3H), 7.33-7.26 (m, 2H), 7.15 (d, J=1.5 Hz, 1H), 7.00 (d, J=1.8 Hz, 1H), 6.63 (d, 0.1=7.5 Hz, 1H), 6.24 (s, 1H), 4.46 (s, 2H), 3.85 (brs, 7H), 3.32 (brs, 4H). MS: 538 (M+

### Example 30

### 4-{[2-(2,6-dichlorobenzyl)-5-oxo-5,6-dihydro-1,6-naphthyridin-4-yl]amino}-N-[4-(dimethylamino)butyl]-3-methoxybenzamide

The title compound was obtained following the procedure described in EXAMPLE 18I, using N¹,N¹-dimethylbutane-1,4-diamine in place of 3-morpholinopropan-1-amine. ¹H NMR (CD₃OD, 300 MHz): δ 12.39 (s, 1H), 7.77 (d, J=7.2 Hz, 1H), 7.52-7.29 (m, 6H), 6.65 (d, J=7.2 Hz, 1H), 6.00 (s, 1H), 4.51 (s, 2H), 3.84 (s, 3H), 3.51-3.45 (m, 2H), 3.3.24-3.18 (m, 2H), 3.15 (s, 3H), 2.91 (s, 3H), 1.84-1.70 (m, 4H). MS: 568 (M+H⁺).

### Example 31

### 2-(2-chlorobenzyl)-4-{[2-methoxy-4-(piperazin-1-yl)phenyl]amino}-1,6-naphthyridin-5(6H)-one

### Example 31A

### tert-butyl 4-(4-(2-chloro-5-oxo-5,6-dihydro-1,6-naphthyridin-4-ylamino)-3-methoxyphenyl)piperazine-1-carboxylate

The title compound was obtained following the procedure described in EXAMPLE 18F, using EXAMPLE 14E in place of EXAMPLE 18B, MS: 486 (M+H⁺).

### Example 31B

### 2-(2-chlorobenzyl)-4-{[2-methoxy-4-(piperazin-1-yl)phenyl]amino}-1,6-naphthyridin-5(6H)-one

A mixture of EXAMPLE 31A (670 mg, 1.38 mmol), 1M (2-chlorobenzyl)zinc(II) bromide in tetrahydrofuran (13.8 mL, 13.8 mmol), tris(dibenzyldeneacetone)dipalladium (O) (126 mg, 0.14 mmol), 2-dicyclohexylphosphino-2′,4′,6′-triisopropylbiphenyl (131 mg, 0.18 mmol) and tetrahydrofuran (5 mL) was degassed with nitrogen for 5 minutes and heated at 110° C. under nitrogen atmosphere for 16 hours. After cooling to ambient temperature and concentration, the residue was purified by preparative HPLC using a gradient of 10/90 to 90/10 acetronitrile in water (containing 0.1% trifluoroacetic acid) to provide the title compound. ¹H NMR (DMSO-d₆, 300 MHz): δ 12.54 (brs, 1H), 11.91 (s, 1H), 8.97 (brs, 2H), 7.84 (t, J=6.6 Hz, 1H), 7.58 (s, 1H), 7.55 (s, 1H), 7.46-7.40 (m, 1H), 7.06 (d, J=8.7 Hz, 1H), 6.70-6.64 (m, 2H), 6.53 (dd, J=2.4, 8.7 Hz, 1H), 5.67 (s, 1H), 4.44 (s, 2H), 3.68 (s, 3H), 3.43 (brs, 4H), 3.28 (brs, 4H). MS: 476 (M+H⁺).

### Example 32

### Enzyme Inhibition Data

The following procedure is used to determine ALK Activity.

ALK kinase assays were conducted with the indicated final concentrations unless otherwise specified. In 384 well black plates (Axygen), 8 μl of compound (2% DMSO) was incubated with 8 μl Lck-peptide substrate (0.5 μM, biotin-Ahx-GAEEEIYAAFFA-COOH) and 8 μl of a mixture of ALK (3 nM, Millipore) and ATP (50 μM) in reaction buffer (50 mM Hepes, pH 7.4; 10 mM MgCl₂; 2 mM MnCl₂; 0.1 mM sodium orthovanadate; 0.01% BSA and 1 mM DTT (added fresh before assay) for 1 h at room temperature. Reactions were then quenched by the addition of 30 μl quench solution (streptavidin-allophycocyanin and Europium-cryptate PT66 monoclonal antibody in 40 mM Hepes, pH 7.4; 480 mM KF; 66 mM EDTA; 0.01% Tween-20; and 0.1% BSA) at room temperature. Plates were read 1 h after quenching on an Envision Multilaber Reader and IC₅₀ values were calculated using a sigmoidal fit of the concentration/inhibition response curves. These values were converted to apparent K, values using the Cheng-Prusoff relationship.

Alternatively, 4 nM ALK (Millipore) and 50 μM ATP were pre-incubated for 30 min at room temperate in 384 well plates (Corning 3676) in 2.5× reaction buffer (125 nM SEB from Cisbio Bioassays, 12.5 mM MgCl₂, 5 mM MnCl₂, and 2.5 mM DTT). Reactions were initiated by the addition of 4 μl ALK-ATP mixture to 2 μl compounds (2% DMSO) and 4 μl TK-substrate biotin (Cisbio Bioassays). After incubation for 1 h at room temperature, reactions were quenched in 10 μl stop buffer (Cisbio detection buffer containing Streptavididn-XL665 and Eu-Cryptate PT66 monoclonal antibody). Plates were read 1 h after quenching on an Envision Multilaber Reader and IC₅₀ values were calculated using a sigmoidal fit of the concentration/inhibition response curves. These values were converted to apparent Kᵢ values using the Cheng-Prusoff relationship. Results are shown in Table 1.

TABLE 1
ALK Activity

| | HTRF_ALK |
|---|---|
| Example | Human - Kᵢ |
| 1 | 0.008 |
| 2 | 0.004 |
| 3 | 0.072 |
| 4 | 0.064 |
| 5 | 0.007 |
| 6 | 0.006 |
| 7 | 0.01 |
| 8 | 0.003 |
| 9 | 0.014 |
| 10 | 0.18 |
| 11 | 0.070 |
| 12 | 0.013 |
| 13 | 0.003 |
| 14 | 0.084 |
| 15 | 1.454 |
| 16 | 0.480 |
| 17 | >1 |
| 18 | 0.051 |
| 19 | 0.025 |
| 20 | 0.051 |
| 21 | 0.025 |
| 22 | 0.054 |
| 23 | 0.012 |
| 24 | 0.008 |
| 25 | 0.022 |
| 26 | 0.212 |
| 27 | 0.017 |
| 28 | 0.036 |
| 29 | 0.011 |
| 30 | 0.020 |
| 31 | 0.007 |

Compounds of the present invention assessed by the above-described assays were found to have ALK kinase-inhibiting activity.

All publication and patent applications cited in this specification are herein incorporated by reference as if each individual publication or patent application were specifically and individually indicated to be incorporated by reference. Although the foregoing invention has been described in some detail byway of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A compound of formula (I): 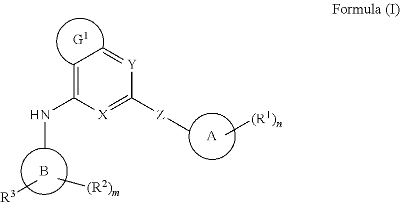
wherein
G¹ is 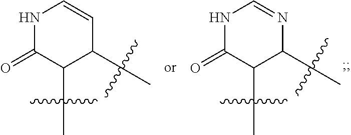 [SMILES: *C1C=CNC(=O)C1* ; *C1N=CNC(=O)C1*]
X is CH or N;
Y is CH or N;
wherein at least one of X and Y is N;
A is phenyl, naphthyl, indenyl, C₃₋₈ cycloalkyl, 5-7 membered heterocycloalkyl, 5-7 membered heterocycloalkenyl, or 5-7 membered heteroaryl;
B is phenyl, pyridinyl, pyrimidinyl, pyrazinyl, pyridazinyl, or pyrazolinyl;
Z is C₁₋₆ alkylene;
R¹, at each occurrence, is independently selected from the group consisting of halo, CN, NO₂, C₁₋₆-alkyl, C₁₋₆-haloalkyl, aryl, C₃₋₈ cycloalkyl, heteroaryl, heterocycloalkyl, OR⁵, SR⁵, C(O)R⁵, C(O)NR⁶R⁷, C(O)OR⁵, OC(O)R⁵, OC(O)NR⁶R⁷, NR⁶R⁷, NR⁶C(O)R⁵, S(O)R⁵, S(O)NR⁶R⁷, S(O)₂R⁵, NR⁶S(O)₂R⁵, and S(O)₂NR⁶R⁷; wherein the C₃₋₈ cycloalkyl, aryl, heterocycloalkyl, and heteroaryl are optionally substituted with 1, 2, or 3 substituents independently selected from halo, C₁₋₄ alkyl, C₁₋₄ haloalkyl, CN, NO₂, OR^{a}, SR^{a}, C(O)R^{a}, C(O)NR^{b}R^{c}, C(O)OR^{a}, OC(O)R^{a}, OC(O)NR^{b}R^{c}, NR^{b}R^{c}, NR^{b}C(O)R^{a}, S(O)R^{a}, S(O)NR^{b}R^{c}, S(O)₂R^{a}, NR^{b}S(O)₂R^{a}, and S(O)₂NR^{b}R^{c};
R², at each occurrence, is independently selected from the group consisting of halo, CN, OH, C₁₋₄ alkyl, C₁₋₄-haloalkyl, C₁₋₄ alkoxy, C₁₋₄ haloalkoxy, C₁₋₄-thioalkoxy, amino, C₁₋₄ alkylamino, and C₁₋₄ dialkylamino;
R³ is selected from the group consisting of aryl, C₃₋₈ cycloalkyl, heteroaryl, heterocycloalkyl, aryl-C₁₋₆-alkyl-, C₃₋₈ cycloalkyl-C₁₋₆-alkyl-, heteroaryl-C₁₋₆-alkyl-, heterocycloalkyl-C₁₋₆-alkyl-, OR⁸, C(O)R⁸, C(O)NR⁹R¹⁰, C(O)OR⁸, OC(O)R⁸, OC(O)NR⁹R¹⁰, NR⁹R¹⁰, NR⁹C(O)R⁸, S(O)R⁸, S(O)NR⁹R¹⁰, S(O)₂R⁸, NR⁹S(O)₂R⁸, and S(O)₂NR⁹R¹⁰, wherein the C₃₋₈ cycloalkyl, aryl, heterocycloalkyl, and heteroaryl, alone or part of another moiety, are optionally substituted with one, two, or three R¹¹;
R⁴ is H or C₁₋₆-alkyl;
R⁵, R⁶, and R⁷, at each occurrence, are independently selected from H, C₁₋₆ alkyl, C₁₋₆ haloalkyl, aryl, C₃₋₈ cycloalkyl, heteroaryl, and heterocycloalkyl, wherein the aryl, C₃₋₈ cycloalkyl, heteroaryl, and heterocycloalkyl moiety are optionally substituted with 1, 2, or 3 substituents independently selected from halo, CN, OH, C₁₋₄ alkyl, C₁₋₄-haloalkyl, C₁₋₄ alkoxy, C₁₋₄haloalkoxy, amino, C₁₋₄ alkylamino, C₁₋₄ dialkylamino, C(O)OH, C(O)C₁₋₄ alkyl, C(O)NH₂, C(O)NH(C₁₋₄ alkyl), or C(O)N(C₁₋₄ alkyl)₂;
R⁸, R⁹, and R¹⁰, at each occurrence, are independently selected from H, C₁₋₆ alkyl, C₁₋₆ haloalkyl, heteroaryl-C₁₋₆-alkyl-, heterocycloalkyl-C₁₋₆-alkyl-, R¹²R¹³N—C₁₋₆-alkyl-, aryl, C₃₋₈ cycloalkyl, heteroaryl, and heterocycloalkyl, wherein the aryl, C₃₋₈ cycloalkyl, heteroaryl, and heterocycloalkyl, alone or as part of another moiety, are optionally substituted with 1, 2, or 3 substituents independently selected from halo, CN, OH, C₁₋₄ alkyl, C₁₋₄-haloalkyl, C₁₋₄ alkoxy, C₁₋₄haloalkoxy, amino, C₁₋₄ alkylamino, C₁₋₄ dialkylamino, C(O)OH, C(O)C₁₋₄ alkyl, C(O)NH₂, C(O)NH(C₁₋₄ alkyl), or C(O)N(C₁₋₄ alkyl)₂;
R¹¹, at each occurrence, is independently selected from the group consisting of halo, C₁₋₄ alkyl, C₁₋₄ haloalkyl, amino-C₁₋₄-alkyl-, C₁₋₄ alkylamino-C₁₋₄ alkyl-, C₁₋₄ dialkylamino-C₁₋₄ alkyl-, hydroxy-C₁₋₄-alkyl-, C₁₋₄ alkyl-C₁₋₄ alkoxy, aryl, C₃₋₈ cycloalkyl, heteroaryl, heterocycloalkyl, aryl-(C₁₋₂ alkyl)-, C₃₋₈ cycloalkyl-(C₁₋₂ alkyl)-, heteroaryl-(C₁₋₂ alkyl)-, heterocycloalkyl-(C₁₋₂ alkyl)-, CN, NO₂, OR^{d}, SR^{d}, C(O)R^{d}, C(O)NR^{e}R^{f}, C(O)OR^{d}, OC(O)R^{d}, OC(O)NR^{e}R^{f}, NR^{e}R^{f}, NR^{e}C(O)R^{d}, S(O)R^{d}, S(O)NR^{e}R^{f}, S(O)₂R^{d}, NR^{e}S(O)₂R^{d}, and S(O)₂NR^{e}R^{f}, wherein the aryl, C₃₋₈ cycloalkyl, heteroaryl, and heterocycloalkyl, alone or as part of another moiety, are optionally substituted with one, two or three substituents independently selected from halo and C₁₋₄ alkyl;
R¹² and R¹³, at each occurrence, are independently selected from the group consisting of H, C₁₋₆ alkyl, C₁₋₆ haloalkyl, aryl, C₃₋₈ cycloalkyl, heteroaryl, and heterocycloalkyl;
R^{a}, at each occurrence, is independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{b} and R^{c}, at each occurrence, are independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{d}, at each occurrence, is independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{e} and R^{f}, at each occurrence, are independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆-alkyl, C₁₋₆-haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{g}, at each occurrence, is independently selected from the group consisting of H, C₁₋₆ alkyl, aryl, C₃₋₈ cycloalkyl, heteroaryl, and heterocycloalkyl; wherein the C₁₋₆-alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆alkyl, C₁₋₆haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
R^{h} and Rⁱ, at each occurrence, are independently selected from the group consisting of H, C₁₋₆ alkyl, aryl, C₃₋₈ cycloalkyl, heteroaryl, and heterocycloalkyl; wherein the C₁₋₆alkyl is optionally substituted with one or more substituents selected from the group consisting of halo, hydroxy, C₁₋₆-alkoxy, —NH₂, —NHC₁₋₆-alkyl, and —N(C₁₋₆-alkyl)₂, and wherein the aryl, C₃₋₈ cycloalkyl, heteroaryl, or heterocycloalkyl is optionally substituted with one or more substituents selected from the group consisting of halo, C₁₋₆alkyl, C₁₋₆haloalkyl, C₁₋₆-hydroxyalkyl, hydroxy, oxo, C₁₋₆-alkoxy, C₁₋₆-haloalkoxy, —NH₂, —NH(C₁₋₆-alkyl), and N(C₁₋₆-alkyl)₂;
m is 0, 1, 2, or 3; and
n is 1, 2, or 3;
or a pharmaceutically acceptable salt or solvate thereof.

2. The compound of claim 1 of formula (I) wherein G¹ is 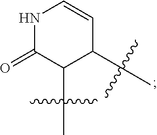 [SMILES: *C1C=CNC(=O)C1*]
X is CH; and
Y is N.

3. The compound of claim 1 of formula (I) wherein G¹ is 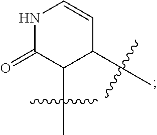 [SMILES: *C1C=CNC(=O)C1*]
X is N; and
Y is CH.

4. The compound of claim 1 of formula (I) wherein G¹ is 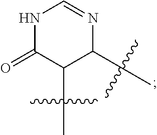 [SMILES: *C1N=CNC(=O)C1*]
X is N; and
Y is CH.

5. The compound of claim 1 of formula (I) wherein Z is —CH₂—.

6. The compound of claim 1 of formula (I) wherein A is phenyl.

7. The compound of claim 6 of formula (I) wherein n is 1, 2, or 3; and R¹ is halo.

8. The compound of claim 1 of formula (I) wherein B is phenyl.

9. The compound of claim 8 of formula (I) wherein R³ is heterocycloalkyl.

10. The compound of claim 8 of formula (I) wherein
m is 0, 1 or 2;
R² is halo, C₁₋₄ alkyl, or C₁₋₄ alkoxy;
R³ is 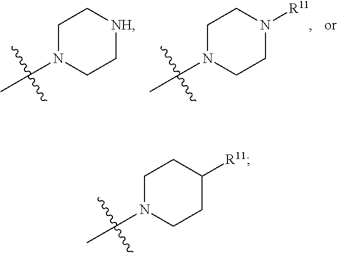
and
R¹¹ is C₁₋₄ alkyl, or NR^{e}R^{f}.

11. The compound of claim 8 of formula (I) wherein
m is 0, 1 or 2;
R² is halo, C₁₋₄ alkyl, or C₁₋₄ alkoxy;
R³ is C(O)R⁸ or C(O)NR⁹R¹⁰;
wherein R⁸, R⁹, and R¹⁰, at each occurrence, are independently selected from H, heterocycloalkyl-C₁₋₆-alkyl-, and R¹²R¹³N—C₁₋₆-alkyl-, wherein the heterocycloalkyl, is optionally substituted with C₁₋₄ alkyl.

12. The compound of claim 1 of formula (I), or a pharmaceutically acceptable salt or solvate thereof, selected from the group consisting of
2-(2,6-dichlorobenzyl)-4-{[2-methoxy-4-(4-methylpiperazin-1-yl)phenyl]amino}-1,6-naphthyridin-5(6H)-one;
2-(2,6-dichlorobenzyl)-4-{[2-methoxy-4-(piperazin-1-yl)phenyl]amino}-1,6-naphthyridin-5(6H)-one;
2-(2,6-dichlorobenzyl)-4-{[4-(piperazin-1-yl)phenyl]amino}-1,6-naphthyridin-5(6H)-one;
6-(2,6-dichlorobenzyl)-8-{[2-methoxy-4-(4-methylpiperazin-1-yl)phenyl]amino}-2,7-naphthyridin-1(2H)-one;
7-(2,6-dichlorobenzyl)-5-{[2-methoxy-4-(4-methylpiperazin-1-yl)phenyl]amino}pyrido[4,3-d]pyrimidin-4 (3H)-one;
7-(2,6-dichlorobenzyl)-5-{[2-methoxy-4-(piperazin-1-yl)phenyl]amino}pyrido[4,3-d]pyrimidin-4(3H)-one;
7-(2,6-dichlorobenzyl)-5-{[4-(piperazin-1-yl)phenyl]amino}pyrido[4,3-d]pyrimidin-4(3H)-one;
6-(2,6-dichlorobenzyl)-8-{[2-methoxy-4-(piperazin-1-yl)phenyl]amino}-2,7-naphthyridin-1(2H)-one;
6-(2,6-dichlorobenzyl)-8-{[4-(piperazin-1-yl)phenyl]amino}-2,7-naphthyridin-1(2H)-one;
6-(2,6-dichlorobenzyl)-8-({4-[4-(dimethylamino)piperidin-1-yl]-2-methoxyphenyl}amino)-2,7-naphthyridin-1(2H)-one;
7-(2,6-dichlorobenzyl)-5-{[2-methoxy-4-(piperazin-1-yl)phenyl]amino}pyrido[2,3-d]pyrimidin-4(3H)-one;
7-(2,6-dichlorobenzyl)-5-{[2-methoxy-4-(piperazin-1-yl)phenyl]amino}pyrido[2,3-d]pyrimidin-4(3H)-one;
-(2,6-dichlorobenzyl)-5-{[2-methoxy-4-(piperazin-1-yl)phenyl]amino}pyrimido[4,5-d]pyrimidin-4(3H)-one;
2-(2,6-dichlorobenzyl)-4-{[3-fluoro-4-(piperazin-1-yl)phenyl]amino}-1,6-naphthyridin-5 (614)-one;
2-(2,6-dichlorobenzyl)-4-{[2-methoxy-4-(piperazin-1-yl)phenyl]amino}pyrido[4,3-d]pyrimidin-5 (6H)-one;
2-(2,6-dichlorobenzyl)-4-{[4-(piperazin-1-yl)phenyl]amino}pyrido[4,3-d]pyrimidin-5 (6H)-one;
6-(2-chloro-6-fluorobenzyl)-8-{[4-(piperazin-1-yl)phenyl]amino}-2,7-naphthyridin-1(2H)-one;
6-(2,6-dichlorobenzyl)-84 {2-methoxy-4-[(4-methylpiperazin-1-yl)carbonyl]phenyl}amino)-2,7-naphthyridin-1(2H)-one;
4-{[2-(2,6-dichlorobenzyl)-5-oxo-5,6-dihydro-1,6-naphthyridin-4-yl]amino}-3-methoxy-N-[3-(morpholin-4-yl)propyl]benzamide;
4-{[3-(2,6-dichlorobenzyl)-8-oxo-7,8-dihydro-2,7-naphthyridin-1-yl]amino}-3-methoxy-N-[3-(morpholin-4-yl)propyl]benzamide;
2-(2,6-dichlorobenzyl)-4-({2-methoxy-4-[(4-methylpiperazin-1-yl)carbonyl]phenyl}amino)-1,6-naphthyridin-5(6H)-one;
4-{[2-(2,6-dichlorobenzyl)-5-oxo-5,6-dihydro-1,6-naphthyridin-4-yl]amino}-3-methoxy-N-[2-(pyrrolidin-1-yl)ethyl]benzamide;
4-{[3-(2,6-dichlorobenzyl)-8-oxo-7,8-dihydro-2,7-naphthyridin-1-yl]amino}-3-methoxy-N-[2-(pyrrolidin-1-yl)ethyl]benzamide;
4-{[2-(2,6-dichlorobenzyl)-5-oxo-5,6-dihydro-1,6-naphthyridin-4-yl]amino}-N-[2-(dimethylamino)ethyl]-3-methoxybenzamide;
4-{[2-(2,6-dichlorobenzyl)-5-oxo-5,6-dihydro-1,6-naphthyridin-4-yl]amino}-3-methoxy-N-[2-(piperidin-1-yl)ethyl]benzamide;
4-{[2-(2,6-dichlorobenzyl)-5-oxo-5,6-dihydro-1,6-naphthyridin-4-yl]amino}-3-methoxy-N-[2-(4-methylpiperazin-1-yl)ethyl]benzamide;
4-{[3-(2,6-dichlorobenzyl)-8-oxo-7,8-dihydro-2,7-naphthyridin-1-yl]amino}-3-methoxy-N-[2-(4-methylpiperazin-1-yl)ethyl]benzamide;
4-{[3-(2,6-dichlorobenzyl)-8-oxo-7,8-dihydro-2,7-naphthyridin-1-yl]amino}-3-methoxy-N-[2-(piperidin-1-yl)ethyl]benzamide;
4-{[3-(2,6-dichlorobenzyl)-8-oxo-7,8-dihydro-2,7-naphthyridin-1-yl]amino}-N-[4-(dimethylamino)butyl]-3-methoxybenzamide;
2-(2,6-dichlorobenzyl)-4-{[2-methoxy-4-(piperazin-1-ylcarbonyl)phenyl]amino}-1,6-naphthyridin-5(6H)-one;
4-{[2-(2,6-dichlorobenzyl)-5-oxo-5,6-dihydro-1,6-naphthyridin-4-yl]amino}-N-[4-(dimethylamino)butyl]-3-methoxybenzamide; and
2-(2-chlorobenzyl)-4-{[2-methoxy-4-(piperazin-1-yl)phenyl]amino}-1,6-naphthyridin-5(6H)-one.

13. A pharmaceutical composition comprising a compound or pharmaceutically acceptable salt of claim 1 and pharmaceutically acceptable excipient.

14. A method of treating cancer in a mammal comprising administering thereto a therapeutically acceptable amount of a compound or pharmaceutically acceptable salt of claim 1.

15. A method for decreasing tumor volume in a mammal comprising administering thereto a therapeutically acceptable amount of a compound or pharmaceutically acceptable salt of claim 1.
